(19) United States
(12) Patent Application Publication
(10) Pub. No.: US 2025/0134992 A1
(43) Pub. Date: May 1, 2025

(54) COMPOSITIONS CONTAINING POLYNUCLEOTIDE AMPHIPHILES AND METHODS OF USE THEREOF

(71) Applicant: Elicio Therapeutics, Inc., Boston, MA (US)

(72) Inventors: Peter C. DEMUTH, Boston, MA (US); Martin P. STEINBUCK, Boston, MA (US)

(21) Appl. No.: 18/684,014

(22) PCT Filed: Aug. 15, 2022
(86) PCT No.: PCT/US2022/040317

§ 371 Date: Feb. 15, 2024

Related U.S. Application Data
(60) Provisional application No. 63/286,952, filed on Dec. 7, 2021.
(60) Provisional application No. 63/233,570, filed on Aug. 16, 2021.

(51) Int. Cl. A61K 39/39; A61K 39/00; A61K 39/215; A61P 31/14; C12N 7/00; C12N 15/117

(52) U.S. Cl. CPC A61K 39/39; A61K 39/215; A61P 31/14; C12N 7/00; C12N 15/117; A61K 2039/55561; A61K 2039/572; A61K 2039/575; C12N 2310/17; C12N 2310/315; C12N 2310/3515; C12N 2770/20034

(57) ABSTRACT

Disclosed herein are polyadenosine, polythymidine, polyguanosine, and/or polycytosine nucleic acid sequences, as well as interferon stimulatory DNA and immunostimulatory herpes simplex virus sequences, that may be conjugated to an albumin-binding domain, as well as pharmaceutically acceptable salts thereof. Furthermore, disclosed herein are methods for inducing an immune response in a subject, and methods of administering the polyadenosine, polythymidine, polyguanosine, and/or polycytosine nucleic acid sequences conjugated to an albumin-binding domain and an antigen to induce an immune response in a subject.

### BACKGROUND OF THE INVENTION

Vaccines are used to stimulate an immune response in an individual to provide protection against and/or treatment for a particular disease. Some vaccines include an antigen to induce an immune response. Immune responses as a result of vaccination have made an enormous contribution to both human and animal health. Since the invention of the first vaccine in 1796, vaccines have come to be considered the most successful method for preventing many infectious diseases by provoking an immune response in a subject. According to the World Health Organization, immunization currently prevents 2-3 million deaths every year across all age groups. The purpose of vaccination is to generate a strong and lasting immune response providing long-term protection against infection. However, many vaccines do not currently induce optimal immunity.

There remains a need to develop new and improved compositions and methods for inducing immune responses in a subject thereof.

### SUMMARY OF THE INVENTION

The disclosure provides compounds, pharmaceutically acceptable salts thereof, pharmaceutical compositions, and kits including a poly-deoxyadenosine (poly-dA) nucleic acid sequence and/or a poly-deoxythymidine (poly-dT) nucleic acid sequence conjugated with an albumin-binding domain, as well as a poly-deoxyguanosine (poly-dG) nucleic acid sequence and/or a poly-deoxycytosine (poly-dC) nucleic acid sequence conjugated with an albumin-binding domain, and pharmaceutically acceptable salts thereof.

The disclosure further provides methods of inducing an immune response in a subject by administering the compounds and salts thereof described herein with an antigen.

In an aspect, the disclosure provides a compound including a poly-deoxyadenosine (poly-dA) nucleic acid sequence and an albumin-binding domain, or a pharmaceutically acceptable salt thereof.

In another aspect, the disclosure provides a compound including a poly-dT nucleic acid sequence and an albumin binding moiety, or a pharmaceutically acceptable salt thereof.

In some embodiments, the compound or pharmaceutically acceptable salt thereof includes a poly-dA nucleic acid sequence and a poly-dT nucleic acid sequence. In some embodiments, the poly-dA nucleic acid sequence and the poly-dT nucleic acid sequence hybridize to form a double-stranded DNA sequence.

In some embodiments, the poly-dA nucleic acid sequence and/or poly-dT nucleic acid sequence include between 30 and 150 nucleotides (e.g., between 30 and 140, 20 and 130, 30 and 120, 30 and 110, 30 and 100, 30 and 90, 30 and 80, 30 and 70, 30 and 60, 30 and 50, 30 and 40, 40 and 150, 50 and 150, 60 and 150, 70 and 150, 80 and 150, 90 and 150,100 and 150,110 and 150,120 and 150,130 and 150, and 140 and 150 nucleotides). In some embodiments, the poly-dA nucleic acid sequence and/or poly-dT nucleic acid sequence include between 30 and 100 nucleotides (e.g., between 30 and 90, 30 and 80, 30 and 70, 30 and 60, 30 and 50, 30 and 40, 40 and 100, 50 and 100, 60 and 100, 70 and 100, 80 and 100, and 90 and 100 nucleotides). In particular embodiments, the poly-dA nucleic acid sequence and/or poly-dT nucleic acid sequence include between 50 and 100 nucleotides (e.g., between 50 and 90, 50 and 80, 50 and 70, 50 and 60, 60 and 100, 70 and 100, 80 and 100, and 90 and 100 nucleotides). In particular embodiments, the poly-dA nucleic acid sequence and/or poly-dT nucleic acid sequence include between 30 and 50 nucleotides (e.g., between 30 and 48, 30 and 46, 30 and 44, 30 and 42, 30 and 40, 30 and 38, 30 and 36, 30 and 34, 30 and 32, 32 and 50, 34 and 50, 36 and 50, 38 and 50, 40 and 50, 42 and 50, 44 and 50, 46 and 50, and 48 and 50 nucleotides). In some embodiments, the poly-dA nucleic acid sequence and/or poly-dT nucleic acid sequence include 30, 35, 40, 45, 50, 55, 65, 70, 75, 80, 85, 90, 95, 100, 105, 110, 115, 120, 125, 130, 135, 140, 145, or 150 nucleotides. In some embodiments, the poly-dA nucleic acid sequence and/or poly-dT nucleic acid sequence include 30, 35, 40, 45, 50, 55, 60, 65, 70, 75, 80, 85, 90, 95, or 100 nucleotides.

In some embodiments, the poly-dA nucleic acid sequence and poly-dT nucleic acid sequence include the same number of nucleotides.

In some embodiments, the poly-dA nucleic acid sequence and/or poly-dT nucleic acid sequence includes a mixture of dA and dT nucleic acid residues. In some embodiments, the poly-dA nucleic acid sequence includes between 100% and 51% (e.g., between 100% and 60%, 100% and 70%, 100% and 80%, 100% and 90%, 100% and 95%, 95% and 51%, 90% and 51%, 80% and 51%, 70% and 51%, and 60% and 51%) dA nucleic acid residues and between 0% and 49% (e.g., 0% and 45%, 0% and 40%, 0% and 30%, 0% and 20%, 0% and 10%, and 0% and 5%, 5% and 49%, 10% and 49%, 20% and 49%, 30% and 49%, and 40% and 49%) dT nucleic acid residues. In some embodiments, the poly-dT nucleic acid sequence includes between 100% and 51% (e.g., between 100% and 60%, 100% and 70%, 100% and 80%, 100% and 90%, 100% and 95%, 95% and 51%, 90% and 51%, 80% and 51%, 70% and 51%, and 60% and 51%) dT nucleic acid residues and between 0% and 49% (e.g., 0% and 45%, 0% and 40%, 0% and 30%, 0% and 20%, 0% and 10%, and 0% and 5%, 5% and 49%, 10% and 49%, 20% and 49%, 30% and 49%, and 40% and 49%) dA nucleic acid residues.

In some embodiments, all internucleotide groups connecting the nucleotides in the poly-dA nucleic acid sequence and poly-dT nucleic acid sequence are phosphodiester or phosphorothioate. In some embodiments, between 50% and 100% (e.g., between 50% and 90%, 50% and 80%, 50% and 70%, 50% and 60%, 60% and 100%, 70% and 100%, 80% and 100%, and 90% and 100%) of the internucleotide groups connecting the nucleotides in the poly-dA nucleic acid sequence and/or poly-dT nucleic acid sequence are phosphorothioate linkages. In some embodiments, between 1 and 10 (e.g., 1, 2, 3, 4, 5, 6, 7, 8, 9, or 10) of the internucleotide groups connecting the nucleotides in the poly-dA nucleic acid sequence and/or poly-dT nucleic acid sequence are phosphodiester linkages and the remaining internucleotide groups connecting the nucleotides of the poly-dA nucleic acid sequence and/or poly-dT nucleic acid sequence are phosphorothioate linkages. In some embodiments, all internucleotide groups connecting the nucleotides in the poly-dA nucleic acid sequence and poly-dT nucleic acid sequence are phosphorothioate.

In some embodiments, the albumin-binding domain is bonded to the 5′ end of the poly-dA nucleic acid sequence. In some embodiments, the albumin-binding domain is bonded to the 5′ end of the poly-dT nucleic acid sequence.

In another aspect, the disclosure provides a compound including an interferon stimulatory DNA (ISD) sequence and an albumin-binding domain, or a pharmaceutically acceptable salt thereof. In some embodiments, the albumin-binding domain is bonded to the 5′ end of the ISD sequence.

In a further aspect, the disclosure provides a compound including an immunostimulatory herpes simplex virus (HSV) sequence and an albumin-binding domain, or a pharmaceutically acceptable salt thereof. In some embodiments, the albumin-binding domain is bonded to the 5′ end of the immunostimulatory HSV sequence. In some embodiments, the albumin-binding domain is bonded to the 5′ end of the sense strand of an immunostimulatory HSV-60 sequence.

In some embodiments, the albumin-binding domain is a lipid. In some embodiments, the lipid is a diacyl lipid. In certain embodiments, the diacyl lipid includes acyl chains including 12-30 hydrocarbon units (e.g., between 12 and 25, 12 and 20, 12 and 15, 15 and 30, 20 and 30, and 25 and 30 hydrocarbon units), 14-25 hydrocarbon units (e.g., between 14 and 22, 14 and 20, 14 and 18, 14 and 16, 16 and 25, 18 and 25, 20 and 25, and 22 and 25 hydrocarbon units), 16-20 hydrocarbon units (e.g., between 16 and 19, 16 and 18, 16 and 17, 17 and 20, 18 and 20, and 19 and 20 hydrocarbon units), or 12, 13, 14, 15, 16, 17, 18, 19, 20, 21, 22, 23, 24, 25, 26, 27, 28, 29, or 30 hydrocarbon units. In some embodiments, the lipid is 1,2-distearoyl-sn-glycero-3-phosphoethanolaminie (DSPE). In some embodiments, the poly-dA nucleic acid sequence, the poly-dT nucleic acid sequence, or the ISD sequence is bonded or linked by a linker to the following lipid:

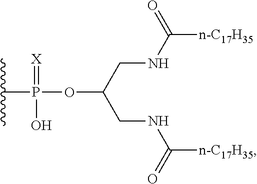

or a salt thereof, wherein X is O or S.

In some embodiments, the linker is selected from the group consisting of a hydrophilic polymer, a string of hydrophilic amino acids, a polysaccharide, and an oligonucleotide, or a combination thereof. In some embodiments, the linker includes “N” polyethylene glycol units, wherein N is between 24-50 (e.g., between 24 and 45, 24 and 40, 24 and 35, 24 and 30, 24 and 26, 26 and 50, 30 and 50, 35 and 50, 40 and 50, and 45 and 50 glycol units). In some embodiments, the linker includes PEG24-amido-PEG24.

In another aspect, the disclosure provides a poly-dA and poly-dT double-stranded DNA sequence including between 30 and 100 paired nucleotides (e.g., 30 and 90, 30 and 80, 30 and 70, 30 and 60, 30 and 50, 30 and 40, 40 and 100, 50 and 100, 60 and 100, 70 and 100, 80 and 100, and 90 and 100), or a pharmaceutically acceptable salt thereof. In some embodiments, the poly-dA and poly-dT include the same number of nucleotides.

In another aspect, the disclosure provides a poly-dA or poly-dT single-stranded DNA sequence including between 30 and 100 nucleotides (e.g., between 30 and 90, 30 and 80, 30 and 70, 30 and 60, 30 and 50, 30 and 40, 40 and 100, 50 and 100, 60 and 100, 70 and 100, 80 and 100, and 90 and 100), or a pharmaceutically acceptable salt thereof.

In an aspect, the disclosure provides a compound including a poly-dG nucleic acid sequence nucleic acid sequence and an albumin-binding domain, or a pharmaceutically acceptable salt thereof.

In another aspect, the disclosure provides a compound including a poly-dC nucleic acid sequence and an albumin binding moiety, or a pharmaceutically acceptable salt thereof.

In some embodiments, the compound or pharmaceutically acceptable salt thereof includes a poly-dG nucleic acid sequence and a poly-dC nucleic acid sequence. In some embodiments, the poly-dG nucleic acid sequence and the poly-dC nucleic acid sequence hybridize to form a double-stranded DNA sequence.

In some embodiments, the poly-dG nucleic acid sequence and/or poly-dC nucleic acid sequence include between 30 and 150 nucleotides (e.g., between 30 and 140, 20 and 130, 30 and 120, 30 and 110, 30 and 100, 30 and 90, 30 and 80, 30 and 70, 30 and 60, 30 and 50, 30 and 40, 40 and 150, 50 and 150, 60 and 150, 70 and 150, 80 and 150, 90 and 150,100 and 150,110 and 150,120 and 150,130 and 150, and 140 and 150 nucleotides). In some embodiments, the poly-dG nucleic acid sequence and/or poly-dC nucleic acid sequence include between 30 and 100 nucleotides (e.g., between 30 and 90, 30 and 80, 30 and 70, 30 and 60, 30 and 50, 30 and 40, 40 and 100, 50 and 100, 60 and 100, 70 and 100, 80 and 100, and 90 and 100 nucleotides). In particular embodiments, the poly-dG nucleic acid sequence and/or poly-dC nucleic acid sequence include between 50 and 100 nucleotides (e.g., between 50 and 90, 50 and 80, 50 and 70, 50 and 60, 60 and 100, 70 and 100, 80 and 100, and 90 and 100 nucleotides). In particular embodiments, the poly-dG nucleic acid sequence and/or poly-dC nucleic acid sequence include between 30 and 50 nucleotides (e.g., between 30 and 48, 30 and 46, 30 and 44, 30 and 42, 30 and 40, 30 and 38, 30 and 36, 30 and 34, 30 and 32, 32 and 50, 34 and 50, 36 and 50, 38 and 50, 40 and 50, 42 and 50, 44 and 50, 46 and 50, and 48 and 50 nucleotides). In some embodiments, the poly-dG nucleic acid sequence and/or poly-dC nucleic acid sequence include 30, 35, 40, 45, 50, 55, 65, 70, 75, 80, 85, 90, 95, 100, 105, 110, 115, 120, 125, 130, 135, 140, 145, or 150 nucleotides. In some embodiments, the poly-dG nucleic acid sequence and/or poly-dC nucleic acid sequence include 30, 35, 40, 45, 50, 55, 60, 65, 70, 75, 80, 85, 90, 95, or 100 nucleotides.

In some embodiments, the poly-dG nucleic acid sequence and poly-dC nucleic acid sequence include the same number of nucleotides.

In some embodiments, the poly-dG nucleic acid sequence and/or poly-dC nucleic acid sequence includes a mixture of dG and dC nucleic acid residues. In some embodiments, the poly-dG nucleic acid sequence includes between 100% and 51% (e.g., between 100% and 60%, 100% and 70%, 100% and 80%, 100% and 90%, 100% and 95%, 95% and 51%, 90% and 51%, 80% and 51%, 70% and 51%, and 60% and 51%) dG nucleic acid residues and between 0% and 49% (e.g., 0% and 45%, 0% and 40%, 0% and 30%, 0% and 20%, 0% and 10%, and 0% and 5%, 5% and 49%, 10% and 49%, 20% and 49%, 30% and 49%, and 40% and 49%) dC nucleic acid residues. In some embodiments, the poly-dC nucleic acid sequence includes between 100% and 51% (e.g., between 100% and 60%, 100% and 70%, 100% and 80%, 100% and 90%, 100% and 95%, 95% and 51%, 90% and 51%, 80% and 51%, 70% and 51%, and 60% and 51%) dC nucleic acid residues and between 0% and 49% (e.g., 0% and 45%, 0% and 40%, 0% and 30%, 0% and 20%, 0% and 10%, and 0% and 5%, 5% and 49%, 10% and 49%, 20% and 49%, 30% and 49%, and 40% and 49%) dG nucleic acid residues.

In some embodiments, all internucleotide groups connecting the nucleotides in the poly-dG nucleic acid sequence and poly-dC nucleic acid sequence are phosphodiester or phosphorothioate. In some embodiments, between 50% and 100% (e.g., between 50% and 90%, 50% and 80%, 50% and 70%, 50% and 60%, 60% and 100%, 70% and 100%, 80% and 100%, and 90% and 100%) of the internucleotide groups connecting the nucleotides in the poly-dG nucleic acid sequence and/or poly-dC nucleic acid sequence are phosphorothioate linkages. In some embodiments, between 1 and 10 (e.g., 1, 2, 3, 4, 5, 6, 7, 8, 9, or 10) of the internucleotide groups connecting the nucleotides in the poly-dG nucleic acid sequence and/or poly-dC nucleic acid sequence are phosphodiester linkages and the remaining internucleotide groups connecting the nucleotides of the poly-dG nucleic acid sequence and/or poly-dC nucleic acid sequence are phosphorothioate linkages. In some embodiments, all internucleotide groups connecting the nucleotides in the poly-dG nucleic acid sequence and poly-dC nucleic acid sequence are phosphorothioate.

In some embodiments, the albumin-binding domain is bonded to the 5′ end of the poly-dG nucleic acid sequence. In some embodiments, the albumin-binding domain is bonded to the 5′ end of the poly-dC nucleic acid sequence.

In some embodiments, the albumin-binding domain is a lipid. In some embodiments, the lipid is a diacyl lipid. In certain embodiments, the diacyl lipid includes acyl chains including 12-30 hydrocarbon units (e.g., between 12 and 25, 12 and 20, 12 and 15, 15 and 30, 20 and 30, and 25 and 30 hydrocarbon units), 14-25 hydrocarbon units (e.g., between 14 and 22, 14 and 20, 14 and 18, 14 and 16, 16 and 25, 18 and 25, 20 and 25, and 22 and 25 hydrocarbon units), 16-20 hydrocarbon units (e.g., between 16 and 19, 16 and 18, 16 and 17, 17 and 20, 18 and 20, and 19 and 20 hydrocarbon units), or 12, 13, 14, 15, 16, 17, 18, 19, 20, 21, 22, 23, 24, 25, 26, 27, 28, 29, or 30 hydrocarbon units. In some embodiments, the lipid is 1,2-distearoyl-sn-glycero-3-phosphoethanolaminie (DSPE). In some embodiments, the poly-dG nucleic acid sequence, the poly-dC nucleic acid sequence, or the ISD sequence is bonded or linked by a linker to the following lipid:

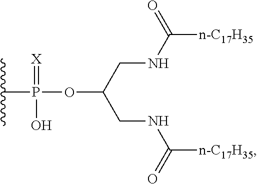

or a salt thereof, wherein X is O or S.

In some embodiments, the linker is selected from the group consisting of a hydrophilic polymer, a string of hydrophilic amino acids, a polysaccharide, and an oligonucleotide, or a combination thereof. In some embodiments, the linker includes “N” polyethylene glycol units, wherein N is between 24-50 (e.g., between 24 and 45, 24 and 40, 24 and 35, 24 and 30, 24 and 26, 26 and 50, 30 and 50, 35 and 50, 40 and 50, and 45 and 50 glycol units). In some embodiments, the linker includes PEG24-amido-PEG24.

In another aspect, the disclosure provides a poly-dG and poly-dC double-stranded DNA sequence including between 30 and 100 paired nucleotides (e.g., 30 and 90, 30 and 80, 30 and 70, 30 and 60, 30 and 50, 30 and 40, 40 and 100, 50 and 100, 60 and 100, 70 and 100, 80 and 100, and 90 and 100), or a pharmaceutically acceptable salt thereof. In some embodiments, the poly-dG and poly-dC include the same number of nucleotides.

In another aspect, the disclosure provides a poly-dG or poly-dC single-stranded DNA sequence including between 30 and 100 nucleotides (e.g., between 30 and 90, 30 and 80, 30 and 70, 30 and 60, 30 and 50, 30 and 40, 40 and 100, 50 and 100, 60 and 100, 70 and 100, 80 and 100, and 90 and 100), or a pharmaceutically acceptable salt thereof.

In another aspect, the disclosure provides a method of inducing an immune response against an antigen in a subject by administering any one of the compounds or pharmaceutically salts thereof described herein and an antigen to the subject. In some embodiments, the method further includes administering an adjuvant to the subject. In some embodiments, the antigen is an influenza antigen, or fragment thereof. In some embodiments, the antigen is an Influenza nucleoprotein, or fragment thereof. For example, the Influenza nucleoprotein may include a polypeptide sequence having at least 85% (e.g., at least 85%, 87%, 88%, 89%, 90%, 91%, 92%, 93%, 94%, 95%, 96%, 97%, 98%, 99%, or 100%) sequence identity to SEQ ID NO: 22, or a fragment thereof. Particularly, the Influenza nucleoprotein may have at least 95% (e.g., at least 96%, 97%, 98%, 99%, or 100%) sequence identity to SEQ ID NO: 22, or a fragment thereof. In certain embodiments, the Influenza nucleoprotein includes a polypeptide sequence of SEQ ID NO: 22, or a fragment thereof.

In some embodiments, the antigen is a coronavirus antigen, or fragment thereof. In some embodiments, the antigen is a coronavirus spike protein, or fragment thereof. In some embodiments, the antigen is a coronavirus nucleocapsid protein, or fragment thereof.

In some embodiments, the antigen is administered intramuscularly, subcutaneously, intravenously, intraperitoneally, topically, or orally. In particular embodiments, any one of the compounds or pharmaceutically salts thereof is administered subcutaneously.

In some embodiments, the subject is a mammal. In some embodiments, the subject is a human.

In another aspect, the disclosure provides a pharmaceutical composition including any one of the compounds or pharmaceutically salts thereof described herein and an antigen, or a nucleic acid sequence encoding the antigen, and a pharmaceutically acceptable carrier. In some embodiments, the antigen is an influenza antigen or fragment thereof. In some embodiments, the antigen is an influenza nucleoprotein or fragment thereof. In some embodiments, the antigen is a coronavirus antigen, or fragment thereof.

In another aspect, the disclosure provides a kit including any one of the compounds or pharmaceutically acceptable salts thereof described herein and an antigen or a nucleic acid sequence encoding the antigen. In some embodiments, the antigen is an influenza antigen, or fragment thereof. In some embodiments, the antigen is an influenza nucleoprotein, or fragment thereof. In some embodiments, the Influenza nucleoprotein comprises a polypeptide sequence having at least 85% (e.g., at least 86%, 87%, 88%, 89%, 90%, 91%, 92%, 93%, 94%, 95%, 96%, 97%, 98%, 99%, or 100%) sequence identity to SEQ ID NO: 22. In some embodiments, the Influenza nucleoprotein comprises a polypeptide sequence having at least 95% (e.g., at least 96%, 97%, 98%, 99%, or 100%) sequence identity to SEQ ID NO: 22. In some embodiments, the Influenza nucleoprotein comprises a polypeptide sequence of SEQ ID NO: 22.

In some embodiments, the antigen is a coronavirus antigen, or fragment thereof.

In each embodiment of an aspect in which a pharmaceutically acceptable salt is referenced, including in the claims, the embodiment may optionally be a pharmaceutically acceptable salt thereof.

### BRIEF DESCRIPTION OF THE DRAWINGS

FIG. 1 are drawings of the double-stranded amphiphilic (AMP) poly-deoxyadenosine (dA) nucleic acid sequence hybridized to a poly-deoxythymidine (dT) nucleic acid sequence (AMP-dAdT), double-stranded AMP-poly-deoxythymidine (dT) nucleic acid sequence hybridized to a poly-deoxyadenosine (dA) nucleic acid sequence (AMP-dTdA), single-stranded AMP-poly-deoxyadenosine (dA), and single-stranded AMP-poly-deoxythymidine (dT).

FIG. 2A and FIG. 2B are graphs showing the splenocyte IFNγ co-culture ELISpot responses of C57Bl6 mice that were administered a vaccine including 5 μg of a coronavirus spike RBD antigen and single-stranded soluble or amphiphilic (AMP) dA or single-stranded soluble or AMP dT (FIG. 2A), and C57Bl6 mice that were administered a vaccine including 5 μg of a coronavirus spike RBD antigen and double-stranded soluble hybridized dA and dT (dA:dT), double-stranded AMP dA:dT where the amphiphile is conjugated to dA, or double-stranded AMP dT:dA where the amphiphile is conjugated to the dT (FIG. 2B). The dA and dT all had a length of 50 nucleotides and only include phosphorothioate (PS).

FIG. 3A and FIG. 3B are graphs showing the percentage of cytokines, including (from top to bottom in each column) IFNγ and TNFα, only TNFα, and only IFNγ, found in peripheral blood cells collected C57BL/6J mice (n=5 per group) that were administered single-stranded soluble or amphiphilic (AMP) dA or single-stranded soluble or AMP dT (FIG. 3A), and C57Bl6 mice that were administered a vaccine including 5 μg of a coronavirus spike RBD antigen and double-stranded soluble hybridized dA and dT (dA:dT), double-stranded AMP dA:dT where the amphiphile is conjugated to dA, or double-stranded AMP dT:dA where the amphiphile is conjugated to the dT (FIG. 3B). The dA and dT all had a length of 50 nucleotides and only include phosphorothioate (PS).

FIG. 3C and FIG. 3D are graphs showing the amount of CD8 cells that are specific to the SARS-CoV-2 RBD antigen isolated from the peripheral blood collected from C57BL/6J mice (n=5 per group) that were administered 5 μg of a coronavirus spike RBD antigen and soluble or amphiphilic single-stranded dA or dT (FIG. 3C), or double-stranded soluble or amphiphilic hybridized dA and dT (dA:dT) or amphiphilic dT:dA (FIG. 3D), where the amphiphile is conjugated to dA or dT. The dA and dT all had a length of 50 nucleotides and only include phosphorothioate (PS).

FIG. 4 is a graph showing the frequency of cytokines in CD4⁺ T cells, including (from top to bottom in each column) IFNγ and TNFα, only TNFα, and only IFNγ, found in peripheral blood cells collected C57BL/6J mice (n=5 per group) that were administered 5 μg of a coronavirus spike RBD antigen and single-stranded soluble or amphiphilic (AMP) dA or single-stranded soluble or AMP, and C57Bl6 mice that were administered a vaccine including 5 μg of a coronavirus spike RBD antigen and double-stranded soluble hybridized dA and dT (dA:dT), double-stranded AMP dA:dT where the amphiphile is conjugated to dA, or double-stranded AMP dT:dA where the amphiphile is conjugated to the dT. The dA and dT all had a length of 50 nucleotides and only include phosphorothioate (PS).

FIG. 5A and FIG. 5B are graphs showing the frequency of intracellular cytokine production, including, from top to bottom in each column, IFNγ and TNFα, only TNFα, and only IFNγ, in CD8⁺ T cells isolated from perfuse lung tissue in C57BL/6J mice (n=5 per group) that were administered a vaccine including 5 μg of a coronavirus spike RBD antigen and single-stranded soluble or amphiphilic (AMP) dA or single-stranded soluble or AMP dT (FIG. 5A), and C57Bl6 mice that were administered a vaccine including 5 μg of a coronavirus spike RBD antigen and double-stranded soluble hybridized dA and dT (dA:dT), double-stranded AMP dA:dT where the amphiphile is conjugated to dA, or double-stranded AMP dT:dA where the amphiphile is conjugated to the dT (FIG. 5B). The dA and dT all had a length of 50 nucleotides and only include phosphorothioate (PS).

FIG. 6A to FIG. 6C are graphs showing the frequency of intracellular cytokine production, including, from top to bottom in each column, IFNγ and TNFα, only TNFα, and only IFNγ, in CD8⁺ T cells isolated from perfuse lung tissue that was restimulated with coronavirus spike peptides from the Wuhan variant (FIG. 6A), from the Alpha variant (FIG. 6B), or from the Beta variant (FIG. 6C) in C57BL/6J mice (n=5 per group) that were administered a vaccine of 5 μg of a coronavirus spike RBD in combination with AMP-dA, AMP-dT, AMP-dA:dT, or AMP-dT:dA. The dA and dT all had a length of 50 nucleotides and only include phosphorothioate (PS) linkages.

FIG. 6D to FIG. 6G are graphs showing the frequency of intracellular cytokine production, including, from top to bottom in each column, IFNγ and TNFα, only TNFα, and only IFNγ, in CD4⁺ T cells isolated from perfuse lung tissue that was restimulated with coronavirus spike peptides from the South African variant pool (FIG. 6D), from the UK variant pool (FIG. 6E), from the custom wildtype pool (FIG. 6F), or from the wildtype Spike pool (FIG. 6G) in C57BL/6J mice (n=5 per group) that were administered a vaccine of 5 μg of a coronavirus spike RBD in combination with soluble or AMP-dA, soluble or AMP-dT, soluble or AMP-dA:dT, or AMP-dT:dA. The dA and dT all had a length of 50 nucleotides and only include phosphorothioate (PS) linkages.

FIG. 6H to FIG. 6J are graphs showing the frequency of intracellular cytokine production, including, from top to bottom in each column, IFNγ and TNFα, only TNFα, and only IFNγ, in CD4⁺ T cells isolated from perfuse lung tissue that was restimulated with coronavirus spike peptides from the Wuhan variant (FIG. 6H), from the Alpha variant pool (FIG. 6I), or from the Beta variant pool (FIG. 6J), in C57BL/6J mice (n=5 per group) that were administered a vaccine of 5 μg of a coronavirus spike RBD in combination with soluble or AMP-dA, soluble or AMP-dT, soluble or AMP-dA:dT, or AMP-dT:dA. The dA and dT all had a length of 50 nucleotides and only include phosphorothioate (PS) linkages.

FIG. 7A and FIG. 7B are graphs showing the frequency of intracellular cytokine production, including, from top to bottom in each column, IFNγ and TNFα, only TNFα, and only IFNγ, in CD4⁺ T cells isolated from perfuse lung tissue in C57BL/6J mice (n=5 per group) that were administered a vaccine including 5 μg of a coronavirus spike RBD antigen and single-stranded soluble or AMP-dA or single-stranded soluble or AMP-dT (FIG. 7A), and C57Bl6 mice that were administered a vaccine including 5 μg of a coronavirus spike RBD antigen and double-stranded soluble hybridized dA and dT (dA:dT), double-stranded AMP dA:dT where the amphiphile is conjugated to dA, or double-stranded AMP dT:dA where the amphiphile is conjugated to the dT (FIG. 7B). The dA and dT all had a length of 50 nucleotides and only include phosphorothioate (PS).

FIGS. 8A and 8B are graphs showing the total radiant efficiency measured in the lymph nodes harvested from C57BL/6J mice (n=20 per group) that were administered a vaccine including 5 μg of the SARS-CoV-2 RBD antigen and 5 nmol of AMP-dT and FAM-dA each 50 nucleotides in length, 5 μg of the SARS-CoV-2 RBD antigen and 5 nmol of dT and FAM-dA each 50 nucleotides in length, 5 μg of the SARS-CoV-2 RBD antigen and 5 nmol of AMP-dT and dA each 50 nucleotides in length either 24 hours after administration (FIG. 8A) or 48 hours after administration (FIG. 8B).

FIG. 9 is a graph showing the frequency of the 6-fluorscein amidite (FAM) fluorophore among cell T cells, macrophages, and dendritic cells in the lymph nodes of C57BL/6J mice (n=20 per group) that were administered a vaccine including 5 μg of the SARS-CoV-2 RBD antigen and 5 nmol of AMP-dT and FAM-dA each 50 nucleotides in length (right side of each panel), including 5 μg of the SARS-CoV-2 RBD antigen and 5 nmol of soluble dA and FAM-dT each 50 nucleotides in length (middle of each panel), or no FAM (left side of each panel) 24 or 48 hours after injection.

FIG. 10A and FIG. 10B are graphs showing the splenocyte IFNγ co-culture ELISpot responses of C57Bl6 mice that were administered a vaccine including 5 μg of the SARS-CoV-2 RBD antigen and soluble dA:dT (FIG. 10A) that is 30, 40, 50, 75, or 100 nucleotides in length or administered a vaccine including 5 μg of the SARS-CoV-2 RBD antigen and AMP-dA:dT (FIG. 10B) that was 30, 40, 50, 75, or 100 nucleotides in length.

FIG. 11A and FIG. 11B are graphs showing the frequency of intracellular cytokine production, including, from top to bottom in each column, IFNγ and TNFα, only TNFα, and only IFNγ, in CD8⁺ T cells from perfuse lung tissue in C57BL/6J mice (n=20 per group) that were administered a vaccine including 5 μg of the SARS-CoV-2 RBD antigen and soluble dA:dT (FIG. 11A) that is 30, 40, 50, 75, or 100 nucleotides in length or administered a vaccine including 5 μg of the SARS-CoV-2 RBD antigen and AMP-dA:dT (FIG. 11B) that was 30, 40, 50, 75, or 100 nucleotides in length.

FIG. 12A and FIG. 12B are graphs showing the frequency of intracellular cytokine production, including, from top to bottom in each column, IFNγ and TNFα, only TNFα, and only IFNγ, in CD8⁺ T cells from perfuse lung tissue in C57BL/6J mice (n=20 per group) that were administered a vaccine including 5 μg of the SARS-CoV-2 RBD antigen and soluble dA:dT (FIG. 12A) that is 30, 40, 50, 75, or 100 nucleotides in length or administered a vaccine including 5 μg of the SARS-CoV-2 RBD antigen and AMP-dA:dT (FIG. 12B) that was 30, 40, 50, 75, or 100 nucleotides in length.

FIG. 13A and FIG. 13B are graphs showing the amount of IgG isolated from the serum of C57Bl6 mice administered a vaccine including 5 μg of the SARS-CoV-2 RBD antigen and soluble dA:dT (FIG. 13A) that is 30, 40, 50, 75, or 100 nucleotides in length or administered a vaccine including 5 μg of the SARS-CoV-2 RBD antigen and AMP-dA:dT (FIG. 13B) that was 30, 40, 50, 75, or 100 nucleotides in length.

FIG. 14A and FIG. 14B are graphs showing the splenocyte IFNγ co-culture ELISpot responses of C57Bl6 mice that were administered a vaccine including 5 μg of the SARS-CoV-2 RBD antigen and either soluble or AMP-dA:dT (FIG. 14A) that is 50 nucleotides in length and has either a PS or PO backbone, or a vaccine 5 μg of the SARS-CoV-2 RBD antigen and either soluble or AMP-ISD (interferon stimulatory DNA) that is 45 nucleotides in length and has either a PS or PO backbone (FIG. 14B).

FIG. 15A and FIG. 15B are graphs showing the frequency of cytokines in CD8⁺ T cells, including (from top to bottom in each column) IFNγ and TNFα, only TNFα, and only IFNγ, found in peripheral blood cells collected from C57BL/6J mice (n=5 per group) that were administered a vaccine including 5 μg of the SARS-CoV-2 RBD antigen and either soluble or AMP-dA:dT (FIG. 15A) that is 50 nucleotides in length and has either a PS or PO backbone, or a vaccine 5 μg of the SARS-CoV-2 RBD antigen and either soluble or AMP-interferon stimulatory DNA (ISD) that is 45 nucleotides in length and has either a PS or PO backbone (FIG. 15B).

FIG. 16A and FIG. 16B are graphs showing the frequency of intracellular cytokine production, including, from top to bottom in each column, IFNγ and TNFα, only TNFα, and only IFNγ, in CD8⁺ T cells isolated from perfuse lung tissue in C57BL/6J mice (n=5 per group) that were administered a vaccine including 5 μg of the SARS-CoV-2 RBD antigen and either soluble or AMP-dA:dT (FIG. 16A) that is 50 nucleotides in length and has either a PS or PO backbone, or a vaccine 5 μg of the SARS-CoV-2 RBD antigen and either soluble or AMP-ISD (interferon stimulatory DNA) that is 45 nucleotides in length and has either a PS or PO backbone (FIG. 16B).

FIG. 17A and FIG. 17B are graphs showing the frequency of intracellular cytokine production, including, from top to bottom in each column, IFNγ and TNFα, only TNFα, and only IFNγ, in CD4⁺ T cells isolated from perfuse lung tissue in C57BL/6J mice (n=5 per group) that were administered a vaccine including 5 μg of the SARS-CoV-2 RBD antigen and either soluble or AMP-dA:dT (FIG. 17A) that is 50 nucleotides in length and has either a PS or PO backbone, or a vaccine 5 μg of the SARS-CoV-2 RBD antigen and either soluble or AMP-ISD (interferon stimulatory DNA) that is 45 nucleotides in length and has either a PS or PO backbone (FIG. 17B).

FIG. 18 is a graph showing the amount of CD8 cells that are specific to the Influenza NP antigen isolated from the peripheral blood collected from C57BL/6J mice (n=5 per group) that were administered a vaccine including 5 μg of an Influenza nucleoprotein antigen and 5 nmol soluble or AMP-CpG or 5 nmol soluble or AMP-dT that is 50 nucleotides in length.

FIG. 19 is a graph showing the frequency of cytokines in CD8⁺ T cells, including (from top to bottom in each column) IFNγ and TNFα, only TNFα, and only IFNγ, found in peripheral blood cells collected C57BL/6J mice (n=5 per group) that were administered a vaccine including 5 μg of an Influenza nucleoprotein antigen and 5 nmol soluble or AMP-CpG or 5 nmol soluble or AMP-dT that is 50 nucleotides in length.

FIG. 20 is a graph showing the splenocyte IFNγ co-culture ELISpot responses of C57Bl6 mice that were administered a vaccine including 5 nmol of double-stranded soluble hybridized dA and dT (dA:dT), double-stranded AMP dA:dT where the amphiphile is conjugated to dA, soluble dT, AMP dT, naked double stranded dA:dT, or 1 nmol of AMP-CpG7090 dT:dA per injection. The dA and dT all had a length of 50 nucleotides and only include phosphorothioate (PS). The naked double stranded dA:dT included a poly(deoxyadenylic-deoxythymidylic) acid sodium salt including a repetitive synthetic double-stranded DNA sequence of poly(dA-dT):poly(dT-dA) and is a synthetic analog of B-DNA.

FIG. 21 is a graph showing the frequency of intracellular cytokine production, including, from top to bottom in each column, IFNγ and TNFα, only TNFα, and only IFNγ, in CD8⁺ T cells isolated from perfuse lung tissue in C57BL/6J mice that were administered a vaccine including 5 nmol of double-stranded soluble hybridized dA and dT (dA:dT), double-stranded AMP dA:dT where the amphiphile is conjugated to dA, soluble dT, AMP dT naked double-stranded dA:dT, or 1 nmol of AMP-CpG7909 per injection, where the naked dA:dT is a poly(deoxyadenylic-deoxythymidylic) acid sodium salt including a repetitive synthetic double-stranded DNA sequence of poly(dA-dT):poly(dT-dA) and is a synthetic analog of B-DNA.

FIG. 22A-FIG. 22C are graphs showing the splenocyte IFNy co-culture ELISpot responses of C57Bl6 mice that were administered a vaccine including 5 μg of a Influenza nucleoprotein (Puerto Rico) and 50 μg alum, 5 nmol of a soluble CpG, 5 nmol of an amphiphilic CpG, 5 nmol of a soluble single-stranded dT, or 5 nmol of an amphiphilic single-stranded dT and that were restimulated with the CDS epitope from the Puerto Rico nucleoprotein (FIG. 22A), from the Ann Arbor nucleoprotein (FIG. 22B), andfrom the Kitakyushu nucleoprotein (FIG. 22C).

FIG. 23 is a graph showing the frequency of intracellular cytokine production, including, from topto bottom in each column, IFNy and TNFa, only TNFa, and only IFNy, in CDS+ T cells isolated from perfuse lung tissue in C57BL/6J mice that were administered a vaccine including 5 μg of Influenza nucleoprotein (Puerto Rico) and 50 μg alum, 5 nmol of a soluble CpG, 5 nmol of an amphiphilic CpG, 5 nmol of a soluble single-stranded dT, or 5 nmol of an amphiphilic single-stranded dT and that were restimulated with the CDS epitope from the Puerto Rico nucleoprotein. The CpG administered was of length of 22 nucleic acids and the dT was of a length of nucleic acids and both included only PS bonds.

FIG. 24A and FIG. 24B are graphs showing the frequency of intracellular cytokine production, including, from top to bottom in each column, IFNy and TNFa, only TNFa, and only IFNy, in CDS+ T cellsisolated from perfuse lung tissue in C57BL/6J mice that were administered a vaccine including 5 μg of Influenza nucleoprotein (Puerto Rico) and 50 μg alum, 5 nmol of a soluble CpG, 5 nmol of an amphiphilic CpG, 5 nmol of a soluble single-stranded dT, or 5 nmol of an amphiphilic single-stranded dT and that were restimulated with the CDS epitope from the Ann Arbor nucleoprotein (FIG. 24A) and from the Kitakyushu nucleoprotein (FIG. 24B). The CpG administered was of length of 22 nucleic acids and the dTwas of a length of nucleic acids and both included only PS bonds.

FIG. 25A and FIG. 25B are graphs showing the frequency of intracellular cytokine production, including, from top to bottom in each column, IFNy and TNFa, only TNFa, and only IFNy, in CD4+ T cells isolated from perfuse lung tissue in C57BL/6J mice that were administered a vaccine including 5 μg of Influenza nucleoprotein (Puerto Rico) and 50 μg alum, 5 nmol of a soluble CpG, 5 nmol of an amphiphilicCpG, 5 nmol of a soluble single-stranded dT, or 5 nmol of an amphiphilic single-stranded dT and that were restimulated with the epitope from the Ann Arbor nucleoprotein (FIG. 25A) and from the Kitakyushunucleoprotein (FIG. 25B). The CpG administered was of length of 22 nucleic acids and the dT was of a length of nucleic acids and both included only PS bonds.

FIG. 26A and FIG. 26B are graphs showing the amount of CD8 cells that are specific to the ovalbumin antigen isolated from the peripheral blood collected from C57BL/6J mice (n=5 per group) that were administered a vaccine including 5 μg of an ovalbumin nucleoprotein antigen and 5 nmol AMP-CpG, 5 nmol soluble or AMP-dA:dT (FIG. 26A); or 5 nmol soluble or AMP-dT (FIG. 26B) that is 50 nucleotides in length.

FIG. 27A and FIG. 27B are graphs showing the frequency of cytokines in CD8⁺ T cells, including (from top to bottom in each column) IFNγ and TNFα, only TNFα, and only IFNγ, found in peripheral blood cells collected from C57BL/6J mice (n=5 per group) that were administered a vaccine including 5 μg of an ovalbumin nucleoprotein antigen and 5 nmol AMP-CpG, 5 nmol soluble or AMP-dA:dT (FIG. 27A); or 5 nmol soluble or AMP-dT (FIG. 27B) that is 50 nucleotides in length.

FIG. 28A and FIG. 28B are graphs showing the frequency of cytokines in CD8⁺ T cells, including, from top to bottom in each column, IFNγ and TNFα, only TNFα, and only IFNγ, isolated from perfuse lung tissue in C57BL/6J mice (n=5 per group) that were administered a vaccine including 5 μg of an ovalbumin nucleoprotein antigen and 5 nmol AMP-CpG, 5 nmol soluble or AMP-dA:dT (FIG. 28A); or 5 nmol soluble or AMP-dT (FIG. 28B) that is 50 nucleotides in length.

FIG. 29A and FIG. 29B are graphs showing the frequency of intracellular cytokine production, including, from top to bottom in each column, IFNγ and TNFα, only TNFα, and only IFNγ, in CD4⁺ T cells isolated from perfuse lung tissue in C57BL/6J mice (n=5 per group) that were administered a vaccine including 5 μg of an ovalbumin antigen and 5 nmol AMP-CpG, 5 nmol soluble or AMP-dA:dT (FIG. 29A); or 5 nmol soluble or AMP-dT (FIG. 29B) that is 50 nucleotides in length.

FIG. 30A and FIG. 30B are graphs showing the splenocyte IFNγ co-culture ELISpot responses of C57Bl6 mice that were administered a vaccine including 5 μg of the ovalbumin antigen and 5 nmol AMP-CpG, 5 nmol soluble or AMP-dA:dT (FIG. 30A); or 5 nmol soluble or AMP-dT (FIG. 30B) that is 50 nucleotides in length nucleotides in length.

FIG. 31A and FIG. 31B are graphs showing the splenocyte IFNγ co-culture ELISpot responses of C57Bl6 mice that were administered a vaccine including 5 μg of the SARS-CoV-2 RBD antigen and 5 nmol single-stranded soluble dT (FIG. 31A) or 5 nmol single-stranded AMP-dT (FIG. 31B) that is 10, 20, 30, 40, 50, 75, or 100 nucleotides in length nucleotides in length and only include phosphorothioate bonds.

FIG. 32A and FIG. 32B are graphs showing the frequency of cytokines in CD8⁺ T cells, including, from top to bottom in each column, IFNγ and TNFα, only TNFα, and only IFNγ, found in peripheral blood cells collected from C57BL/6J mice (n=5 per group) that were administered a vaccine including 5 μg of the SARS-CoV-2 RBD antigen and 5 nmol single-stranded soluble dT (FIG. 32A) or 5 nmol single-stranded AMP-dT (FIG. 32B) that is 10, 20, 30, 40, 50, 75, or 100 nucleotides in length nucleotides in length and only include phosphorothioate bonds.

FIG. 33A and FIG. 33B are graphs showing the frequency of cytokines in CD8⁺ T cells, including, from top to bottom in each column, IFNγ and TNFα, only TNFα, and only IFNγ, isolated from perfuse lung tissue collected from C57BL/6J mice (n=5 per group) that were administered a vaccine including 5 μg of the SARS-CoV-2 RBD antigen and 5 nmol single-stranded soluble dT (FIG. 33A) or 5 nmol single-stranded AMP-dT (FIG. 33B) that is 10, 20, 30, 40, 50, 75, or 100 nucleotides in length nucleotides in length and only include phosphorothioate bonds.

FIG. 34A and FIG. 34B are graphs showing the frequency of intracellular cytokine production, including, from top to bottom in each column, IFNγ and TNFα, only TNFα, and only IFNγ, in CD4⁺ T cells isolated from perfuse lung tissue in C57BL/6J mice (n=5 per group) that were administered a vaccine including 5 μg of the SARS-CoV-2 RBD antigen and 5 nmol single-stranded soluble dT (FIG. 34A) or 5 nmol single-stranded AMP-dT (FIG. 34B) that is 10, 20, 30, 40, 50, 75, or 100 nucleotides in length nucleotides in length and only include phosphorothioate bonds.

FIG. 35 is a graph showing the amount of CD8 cells that are specific to the ovalbumin antigen isolated from the peripheral blood collected from C57BL/6J mice (n=5 per group) that were administered a vaccine including 5 μg of an ovalbumin nucleoprotein antigen and 5 nmol alum, IFA, MF59, ASO3, ASO4, AMP-dA:dT, AMP-dT, or AMP-CpG.

FIG. 36 is a graph showing the splenocyte IFNγ co-culture ELISpot responses of C57Bl6 mice that were administered a vaccine including 5 μg of the ovalbumin antigen and 5 nmol alum, IFA, MF59, ASO3, ASO4, AMP-dA:dT, AMP-dT, or AMP-CpG.

FIG. 37 is a graph showing the frequency of cytokines in CD8⁺ T cells, including, from top to bottom in each column, IFNγ and TNFα, only TNFα, and only IFNγ, isolated from peripheral blood cells collected from C57BL/6J mice (n=5 per group) that were administered a vaccine including 5 μg of the ovalbumin antigen and 5 nmol alum, IFA, MF59, ASO3, ASO4, AMP-dA:dT, AMP-dT, or AMP-CpG.

FIG. 38 is a graph showing the frequency of intracellular cytokine production, including, from top to bottom in each column, IFNγ and TNFα, only TNFα, and only IFNγ, in CD4⁺ T cells isolated from peripheral blood cells in C57BL/6J mice (n=5 per group) that were administered a vaccine including 5 μg of the ovalbumin antigen and 5 nmol alum, IFA, MF59, ASO3, ASO4, AMP-dA:dT, AMP-dT, or AMP-CpG.

FIG. 39 is a graph showing the frequency of cytokines in CD8⁺ T cells, including, from top to bottom in each column, IFNγ and TNFα, only TNFα, and only IFNγ, isolated from perfuse lung tissue collected from C57BL/6J mice (n=5 per group) that were administered a vaccine including 5 μg of the ovalbumin antigen and 5 nmol alum, IFA, MF59, AS03, AS04, AMP-dA:dT, AMP-dT, or AMP-CpG.

FIG. 40 is a graph showing the frequency of intracellular cytokine production, including, from top to bottom in each column, IFNγ and TNFα, only TNFα, and only IFNγ, in CD4⁺ T cells isolated from perfuse lung tissue in C57BL/6J mice (n=5 per group) that were administered a vaccine including 5 μg of the ovalbumin antigen and 5 nmol alum, IFA, MF59, AS03, AS04, AMP-dA:dT, AMP-dT, or AMP-CpG.

FIG. 41 is a graph showing the amount of anti-ovalbumin IgG isolated from the serum of C57BL/6J mice (n=5 per group) that were administered a vaccine including 5 μg of the ovalbumin antigen and 5 nmol alum, IFA, MF59, AS03, AS04, AMP-dA:dT, AMP-dT, or AMP-CpG.

FIG. 42A and FIG. 42B are graphs showing the amount of anti-ovalbumin IgG1 (FIG. 42A) and IgG2c (FIG. 42B) isolated from the serum of C57BL/6J mice (n=5 per group) that were administered a vaccine including 5 μg of the ovalbumin antigen and 5 nmol alum, IFA, MF59, ASO3, ASO4, AMP-dA:dT, AMP-dT, or AMP-CpG.

FIG. 42C is a graph showing the ratio of anti-ovalbumin IgG2c to IgG1 isolated from the serum of C57BL/6J mice (n=5 per group) that were administered a vaccine including 5 μg of the ovalbumin antigen and 5 nmol alum, IFA, MF59, AS03, AS04, AMP-dA:dT, AMP-dT, or AMP-CpG.

FIG. 43 is a schematic diagram showing the timeline of vaccination of the adjuvant and SARS-CoV-2 Spike antigen in Rhesus macaques and the sample collection timeline.

FIG. 44A is a graph showing the amount of IgG isolated from the serum of Rhesus macaque monkeys administered a vaccine including 3000 μg AMP-CpG and SARS-CoV-2 Spike RBD.

FIG. 44B is a graph showing the pseudovirus neutralization titers 50 for serum from Rhesus macaque monkeys administered a vaccine including 3000 μg AMP-CpG and SARS-CoV-2 Spike RBD compared to convalescent human sera.

FIG. 45 is a series of graphs showing the amount of CD8 cells that are specific to the RBD antigen isolated from the peripheral blood collected from Rhesus macaques that were administered a vaccine including an adjuvant of amphiphilic CpG7909 or amphiphilic poly-dT 50 nucleotides in length, and an antigen including SARS-CoV-2 Spike RBD, Variant of Concern (VOC) Spike RBD, or SARS-CoV-2 Spike.

FIG. 46 is a graph showing the amount of IgG isolated from the serum of Rhesus macaque monkeys administered a vaccine including (left to right; Baseline to Week 8) 5000 μg AMP-CpG and SARS-CoV-2 Spike RBD, 10000 μg of AMP-CpG and SARS-CoV-2 Spike RBD, 5000 μg of AMP CpG and VOC Spike RBD, 5000 μg of AMP-CpG, or 5000 μg AMP-CpG SARS-CoV-2 Spike antigen, where the SARS-CoV-2 Spike antigen is from the Wuhan variant.

FIG. 47 is a graph showing the amount of IgG isolated from the serum of Rhesus macaque monkeys administered a vaccine including (left to right; Baseline to Week 8) 5000 μg AMP-CpG and SARS-CoV-2 Spike RBD, 10000 μg of AMP-CpG and SARS-CoV-2 Spike RBD, 5000 μg of AMP CpG and VOC Spike RBD, 5000 μg of AMP-CpG, or 5000 μg AMP-CpG SARS-CoV-2 Spike antigen, where the SARS-CoV-2 Spike antigen is from the Delta variant.

FIG. 48 is a graph showing the amount of IgG isolated from the serum of Rhesus macaque monkeys administered a vaccine including (left to right; Baseline to Week 8) 5000 μg AMP-CpG and SARS-CoV-2 Spike RBD, 10000 μg of AMP-CpG and SARS-CoV-2 Spike RBD, 5000 μg of AMP CpG and VOC Spike RBD, 5000 μg of AMP-CpG, or 5000 μg AMP-CpG SARS-CoV-2 Spike antigen, where the SARS-CoV-2 Spike antigen is from the Beta variant.

FIG. 49 is a graph showing the amount of IgG isolated from the serum of Rhesus macaque monkeys administered a vaccine including 5000 μg AMP-CpG and SARS-CoV-2 Spike RBD, 10000 μg of AMP-CpG and SARS-CoV-2 Spike RBD, 5000 μg of AMP CpG and VOC Spike RBD, 5000 μg of AMP-CpG, or 5000 μg AMP-CpG SARS-CoV-2 Spike antigen, after 6 weeks for each of the SARS-CoV-2 Spike antigens examined.

FIG. 50A-FIG. 50D are graphs showing the concentration of intracellular cytokine production of IFNγ (FIG. 50A), IL-6 (FIG. 50B), IL-1 RA (FIG. 50C), and IL-18 (FIG. 50D) from of Rhesus macaque monkeys that were administered a vaccine including (from left to right at each timepoint) 5000 μg AMP-CpG and SARS-CoV-2 Spike RBD, 10000 μg of AMP-CpG and SARS-CoV-2 Spike RBD, 5000 μg of AMP CpG and VOC Spike RBD, 5000 μg of AMP-CpG, or 5000 μg AMP-CpG SARS-CoV-2 Spike antigen.

FIG. 51 is a series of graphs showing the splenocyte IFNγ co-culture ELISpot responses of Rhesus macaque monkeys that were administered a vaccine including (from left to right) 5000 μg AMP-CpG and SARS-CoV-2 Spike RBD, 10000 μg of AMP-CpG and SARS-CoV-2 Spike RBD, 5000 μg of AMP CpG and VOC Spike RBD, 5000 μg of AMP-CpG, or 5000 μg AMP-CpG SARS-CoV-2 Spike antigen, where the SARS-CoV-2 Spike antigen is from the Wuhan variant.

FIG. 52 is a series of graphs showing the splenocyte IFNγ co-culture ELISpot responses of Rhesus macaque monkeys that were administered a vaccine including (from left to right) 5000 μg AMP-CpG and SARS-CoV-2 Spike RBD, 10000 μg of AMP-CpG and SARS-CoV-2 Spike RBD, 5000 μg of AMP CpG and VOC Spike RBD, 5000 μg of AMP-CpG, or 5000 μg AMP-CpG SARS-CoV-2 Spike antigen, where the SARS-CoV-2 Spike antigen is from the Delta variant.

FIG. 53 is a series of graphs showing the splenocyte IFNγ co-culture ELISpot responses of Rhesus macaque monkeys that were administered a vaccine including (from left to right) 5000 μg AMP-CpG and SARS-CoV-2 Spike RBD, 10000 μg of AMP-CpG and SARS-CoV-2 Spike RBD, 5000 μg of AMP CpG and VOC Spike RBD, 5000 μg of AMP-CpG, or 5000 μg AMP-CpG SARS-CoV-2 Spike antigen, where the SARS-CoV-2 Spike antigen is from the Beta variant.

FIG. 54 are drawings of the double-stranded amphiphilic poly-deoxyadenosine (AMP-dA) nucleic acid sequence hybridized to a poly-deoxythymidine nucleic acid sequence (dT), the double-stranded amphiphilic alternating poly-deoxyadenosine and poly-deoxythymidine (AMP-dAdT) nucleic acid sequence hybridized to a complementary alternating poly-deoxyadenosine and poly-deoxythymidine (dAdT) nucleic acid sequence, and a double-stranded amphiphilic ISD nucleic acid sequence having phosphorothioate bonds (AMP-ISD-PS) hybridized to a complementary ISD nucleic acid sequence having phosphorothioate bonds.

FIG. 55 is a graph showing the splenocyte IFNγ co-culture ELISpot responses of C57Bl6 mice 21 days after they were administered two doses of a vaccine including 5 nmol of double-stranded soluble hybridized dA:dT, double-stranded hybridized, amphiphilic dA:dT, double-stranded soluble alternating dAdT:dAdT, double-stranded amphiphilic alternating dAdT:dAdT, double-stranded soluble ISD, and double-stranded amphiphilic ISD, and 5 μg of Spike RBD on day 0 and day 14.

FIG. 56A and FIG. 56B are graphs showing the percentage of cytokines, including (from top to bottom in each column) IFNγ and TNFα, only TNFα, and only IFNγ, found in CD8⁺ T cells (FIG. 56A) or CD4⁺ T cells (FIG. 56B) isolated from peripheral blood cells collected from C57BL/6J mice 21 days after they were administered two doses of a vaccine including 5 nmol of double-stranded soluble hybridized dA:dT, double-stranded hybridized, amphiphilic dA:dT, double-stranded soluble alternating dAdT:dAdT, double-stranded amphiphilic alternating dAdT:dAdT, double-stranded soluble ISD, and double-stranded amphiphilic ISD, and 5 μg of Spike RBD on day 0 and day 14.

FIG. 57A and FIG. 57B are graphs showing the percentage of cytokines, including (from top to bottom in each column) IFNγ and TNFα, only TNFα, and only IFNγ, found in CD8⁺ T cells (FIG. 57A) or CD4⁺ T cells (FIG. 57B) isolated from lung tissue collected from C57BL/6J mice 21 days after they were administered two doses of a vaccine including 5 nmol of double-stranded soluble hybridized dA:dT, double-stranded hybridized, amphiphilic dA:dT, double-stranded soluble alternating dAdT:dAdT, double-stranded amphiphilic alternating dAdT:dAdT, double-stranded soluble ISD, and double-stranded amphiphilic ISD, and 5 μg of Spike RBD on day 0 and day 14.

FIG. 58 shows the transcriptomic data of various genes from tissue extracted from the lymph nodes of mice 2 hours after they were administered double-stranded amphiphilic dAdT, double-stranded soluble dAdT, single stranded amphiphilic dT, single-stranded soluble dT, amphiphilic CpG, and soluble CpG and 5 μg of Spike RBD.

FIG. 59 shows the transcriptomic data of various genes from tissue extracted from the lymph nodes of mice 6 hours after they were administered double-stranded amphiphilic dAdT, double-stranded soluble dAdT, single stranded amphiphilic dT, single-stranded soluble dT, amphiphilic CpG, and soluble CpG and 5 μg of Spike RBD.

FIG. 60 shows the transcriptomic data of various genes from tissue extracted from the lymph nodes of mice 24 hours after they were administered double-stranded amphiphilic dAdT, double-stranded soluble dAdT, single stranded amphiphilic dT, single-stranded soluble dT, amphiphilic CpG, and soluble CpG and 5 μg of Spike RBD.

FIG. 61 shows the transcriptomic data of various genes from tissue extracted from the lymph nodes of mice 72 hours after they were administered double-stranded amphiphilic dAdT, double-stranded soluble dAdT, single stranded amphiphilic dT, single-stranded soluble dT, amphiphilic CpG, and soluble CpG and 5 μg of Spike RBD.

FIG. 62 are drawings of the double-stranded amphiphilic Herpes Simplex virus (AMP-HSV60) nucleic acid sequence hybridized to a complementary HSV60 nucleic acid sequence and a single-stranded amphiphilic HSV60 (AMP-HSV60-Sense).

FIG. 63 is a graph showing the splenocyte IFNγ co-culture ELISpot responses of C57Bl6 mice 21 days after they were administered two doses of a vaccine including 5 nmol of double-stranded soluble hybridized HSV60, double-stranded hybridized, amphiphilic HSV60, single-stranded soluble HSV60, and 5 μg of Spike RBD on day 0 and day 14.

FIG. 64A and FIG. 64B are graphs showing the percentage of cytokines, including (from top to bottom in each column) IFNγ and TNFα, only TNFα, and only IFNγ, found in CD8⁺ T cells (FIG. 64A) or CD4⁺ T cells (FIG. 64B) isolated from peripheral blood cells collected from C57BL/6J mice 21 days after they were administered two doses of a vaccine including 5 nmol of double-stranded soluble hybridized HSV60, double-stranded hybridized, amphiphilic HSV60, single-stranded soluble HSV60, and 5 μg of Spike RBD on day 0 and day 14.

FIG. 65A and FIG. 65B are graphs showing the percentage of cytokines, including (from top to bottom in each column) IFNγ and TNFα, only TNFα, and only IFNγ, found in CD8⁺ T cells (FIG. 65A) or CD4⁺ T cells (FIG. 65B) isolated from lung tissue collected from C57BL/6J mice 21 days after they were administered two doses of a vaccine including 5 nmol of double-stranded soluble hybridized HSV60, double-stranded hybridized, amphiphilic HSV60, single-stranded soluble HSV60, and 5 μg of Spike RBD on day 0 and day 14.

FIG. 66 is graph showing the amount of CD8⁺ cells that are specific to the SARS-CoV-2 RBD antigen isolated from the peripheral blood collected from C57BL/6J 21 days after they were administered two doses of a vaccine including 5 nmol of double-stranded soluble hybridized HSV60, double-stranded hybridized, amphiphilic HSV60, single-stranded soluble HSV60, and 5 μg of Spike RBD on day 0 and day 14.

FIG. 67 are drawings of the double-stranded amphiphilic ISD (AMP-ISD) nucleic acid sequence hybridized to a complementary ISD nucleic acid sequence of a single-stranded amphiphilic ISD (AMP-ISD-Sense or AMP-ISD-Anti-Sense).

FIG. 68 is a graph showing the splenocyte IFNγ co-culture ELISpot responses of C57Bl6 mice 21 days after they were administered two doses of a vaccine including 5 nmol of double-stranded soluble hybridized ISD, double-stranded hybridized, amphiphilic ISD, single-stranded soluble sense ISD, single-stranded amphiphilic sense ISD, single-stranded soluble anti-sense ISD, single-stranded amphiphilic anti-sense ISD, and 5 μg of Spike RBD on day 0 and day 14.

FIG. 69A and FIG. 69B are graphs showing the percentage of cytokines, including (from top to bottom in each column) IFNγ and TNFα, only TNFα, and only IFNγ, found in CD8⁺ T cells (FIG. 69A) or CD4⁺ T cells (FIG. 69B) isolated from peripheral blood cells collected from C57BL/6J mice 21 days after they were administered two doses of a vaccine including 5 nmol of double-stranded soluble hybridized ISD, double-stranded hybridized, amphiphilic ISD, single-stranded soluble sense ISD, single-stranded amphiphilic sense ISD, single-stranded soluble anti-sense ISD, single-stranded amphiphilic anti-sense ISD, and 5 μg of Spike RBD on day 0 and day 14.

FIG. 70A and FIG. 70B are graphs showing the percentage of cytokines, including (from top to bottom in each column) IFNγ and TNFα, only TNFα, and only IFNγ, found in CD8⁺ T cells (FIG. 70A) or CD4⁺ T cells (FIG. 70B) isolated from lung tissue collected from C57BL/6J mice 21 days after they were administered two doses of a vaccine including 5 nmol of double-stranded soluble hybridized ISD, double-stranded hybridized, amphiphilic ISD, single-stranded soluble sense ISD, single-stranded amphiphilic sense ISD, single-stranded soluble anti-sense ISD, single-stranded amphiphilic anti-sense ISD, and 5 μg of Spike RBD on day 0 and day 14

### DEFINITIONS

Terms used in the claims and specification are defined as set forth below unless otherwise specified.

It must be noted that, as used in the specification and the appended claims, the singular forms “a,” “an,” and “the” include plural referents unless the context clearly dictates otherwise.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

As used herein, the term “adjuvant” refers to a compound that, with a specific immunogen or antigen, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses. In certain embodiments, the adjuvant is a cyclic dinucleotide. In some embodiments, the adjuvant is an immunostimulatory oligonucleotide as described herein.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid. Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions,” can be made, e.g., by insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

As used herein, “amphiphile” or “amphiphilic” refers to a conjugate comprising a hydrophilic head group and a hydrophobic tail, thereby forming an amphiphilic conjugate. In some embodiments, an amphiphile conjugate comprises a poly-dA and/or poly-dT sequence and one or more hydrophobic lipid tails.

The term “ameliorating” refers to any therapeutically beneficial result in the treatment of a disease state, e.g., influenza and SARS-CoV-2, including prophylaxis, lessening in the severity or progression, remission, or cure thereof.

As used herein, “cancer antigen” refers to (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.

A polypeptide or amino acid sequence “derived from” a designated polypeptide or protein or a “polypeptide fragment” refers to the origin of the polypeptide. Preferably, the polypeptide or amino acid sequence which is derived or is a fragment of is from a particular sequence that has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence. Polypeptides derived from or that are fragments of another peptide may have one or more mutations relative to the starting polypeptide, e.g., one or more amino acid residues which have been substituted with another amino acid residue or which has one or more amino acid residue insertions or deletions.

A polypeptide can comprise an amino acid sequence which is not naturally occurring. Such variants necessarily have less than 100% sequence identity or similarity with the starting molecule. In a preferred embodiment, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

In one embodiment, there is one amino acid difference between a starting polypeptide sequence and the sequence derived therefrom. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity.

As used herein, the term “cytotoxic T lymphocyte (CTL) response” refers to an immune response induced by cytotoxic T cells. CTL responses are mediated primarily by CD8⁺ T cells.

As used herein, the term “effective dose” or “effective dosage” is defined as an amount sufficient to achieve or at least partially achieve the desired effect.

The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

As used herein, “immune cell” is a cell of hematopoietic origin and that plays a role in the immune response. Immune cells include lymphocytes (e.g., B cells and T cells), natural killer cells, and myeloid cells (e.g., monocytes, macrophages, eosinophils, mast cells, basophils, and granulocytes). In particular embodiments, the immune cell is T cell.

As used herein, “immune response” refers to a response made by the immune system of an organism to a substance, which includes but is not limited to foreign or self proteins. Three general types of “immune response” include mucosal, humoral, and cellular immune responses. For example, the immune response can include the activation, expansion, and/or increased proliferation of an immune cell. An immune response may also include at least one of the following: cytokine production, T cell activation and/or proliferation, granzyme or perforin production, activation of antigen presenting cells or dendritic cells, antibody production, inflammation, developing immunity, developing hypersensitivity to an antigen, the response of antigen-specific lymphocytes to antigen, clearance of an infectious agent, and transplant or graft rejection.

As used herein, an “immunostimulatory oligonucleotide” is an oligonucleotide that can stimulate (e.g., induce or enhance) an immune response.

The terms “inducing an immune response” and “enhancing an immune response” are used interchangeably and refer to the stimulation of an immune response (i.e., either passive or adaptive) to a particular antigen.

The term “induce” as used with respect to inducing complement dependent cytotoxicity (CDC) or antibody-dependent cellular cytotoxicity (ADCC) refer to the stimulation of particular direct cell killing mechanisms.

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment (such as treatment with a composition comprising an amphiphilic ligand conjugate).

The term “in vivo” refers to processes that occur in a living organism.

The term “in vitro” refers to processes that occur outside a living organism, such as in a test tube, flask, or culture plate.

As used herein, the terms “linked,” “operably linked,” “fused,” or “fusion,” are used interchangeably. These terms refer to the joining together of two more elements or components or domains, by an appropriate means including chemical conjugation or recombinant DNA technology. Methods of chemical conjugation (e.g., using heterobifunctional crosslinking agents) are known in the art as are methods of recombinant DNA technology.

The term “lipid” refers to a biomolecule that is soluble in nonpolar solvents and insoluble in water. Lipids are often described as hydrophobic or amphiphilic molecules which allows them to form structures such as vesicles or membranes in aqueous environments. Lipids include fatty acids, glycerolipids, glycerophospholipids, sphingolipids, sterol lipids (including cholesterol), prenol lipids, saccharolipids, and polyketides. In some embodiments, the lipid suitable for the amphiphilic ligand conjugates of the disclosure binds to human serum albumin under physiological conditions. In some embodiments, the lipid suitable for the amphiphilic ligand conjugates of the disclosure inserts into a cell membrane under physiological conditions. In some embodiments, the lipid binds albumin and inserts into a cell membrane under physiological conditions. In some embodiments, the lipid is a diacyl lipid. In some embodiments, the diacyl lipid includes at least 12 carbons. In some embodiments, the diacyl lipid includes 12-30 hydrocarbon units, 14-25 hydrocarbon units, or 16-20 hydrocarbon units. In some embodiments, the diacyl lipid includes 12, 13, 14, 15, 16, 17, 18, 19, 20, 21, 22, 23, 24, 25, 26, 27, 28, 29 or 30 carbons.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985); and Cassol et al., 1992; Rossolini et al., Mal. Cell. Probes 8:91-98, 1994). For arginine and leucine, modifications at the second base can also be conservative. The term nucleic acid is used interchangeably with gene, cDNA, and mRNA encoded by a gene.

Polynucleotides of the present invention can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms. In some embodiments, the peptides of the invention are encoded by a nucleotide sequence. Nucleotide sequences of the invention can be useful for a number of applications, including: cloning, gene therapy, protein expression and purification, mutation introduction, DNA vaccination of a host in need thereof, antibody generation for, e.g., passive immunization, PCR, primer and probe generation, and the like.

As used herein, “parenteral administration,” “administered parenterally,” and other grammatically equivalent phrases, refer to modes of administration other than enteral and topical administration, usually by injection, and include, without limitation, intravenous, intranasal, intraocular, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intracerebral, intracranial, intracarotid and intrasternal injection and infusion.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

The term “pharmaceutically acceptable salt,” as used herein, means any pharmaceutically acceptable salt of a conjugate, oligonucleotide, or peptide disclosed herein. Pharmaceutically acceptable salts of any of the compounds and nucleic acid sequences described herein may include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use (Eds. P. H. Stahl and C. G. Wermuth), Wiley-VCH, 2008. The salts can be prepared in situ during the final isolation and purification of the compounds described herein or separately by reacting a free base group with a suitable acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. References to the compounds, nucleic acids, conjugates, oligonucleotides, or peptides in the claims and elsewhere herein optionally include pharmaceutically acceptable salts thereof unless otherwise indicated or not applicable.

As used herein, the term “physiological conditions” refers to the in vivo condition of a subject. In some embodiments, physiological condition refers to a neutral pH (e.g., pH between 6-8).

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “subject” or “mammal” or “patient” includes any human or non-human animal. For example, the methods and compositions of the present invention can be used to treat a subject with a disease or condition. The term “non-human animal” includes all vertebrates, e.g., mammals and non-mammals, such as non-human primates, sheep, dogs, cats, mice, horses, pigs, cows, chickens, amphibians, reptiles, etc.

The term “sufficient amount” or “amount sufficient to” means an amount sufficient to produce a desired effect, e.g., an amount sufficient to reduce the diameter of a tumor.

The term “T cell” refers to a type of white blood cell that can be distinguished from other white blood cells by the presence of a T cell receptor on the cell surface. There are several subsets of T cells, including, but not limited to, T helper cells (a.k.a. T_{H} cells or CD4⁺ T cells) and subtypes, including T_{H}, T_{H}2, T_{H}3, T_{H}17, T_{H}9, and T_{FH} cells, cytotoxic T cells (i.e., Tc cells, CD8⁺ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. Treg cells or suppressor T cells) and subtypes, including CD4⁺FOXP3⁺ T_{reg} cells, CD4⁺FOXP3⁻ T_{reg} cells, Tr1 cells, Th3 cells, and T_{reg}17 cells, natural killer T cells (a.k.a. NKT cells), mucosal associated invariant T cells (MAITs), and gamma delta T cells (γδ T cells), including Vγ9/Vδ2 T cells. Any one or more of the aforementioned or unmentioned T cells may be the target cell type for a method of use of the invention.

The terms “treat,” “treating,” and “treatment,” as used herein, refer to therapeutic or preventative measures described herein. The methods of “treatment” employ administration to a subject, in need of such treatment, a poly-dA nucleic acid sequence and/or poly-dT nucleic acid sequence and an albumin-binding domain of the present disclosure. In some embodiments, a poly-dA nucleic acid sequence, poly-dT, poly-dG, and/or poly-dC nucleic acid sequence conjugated to an albumin-binding domain is administered to a subject in need of an enhanced immune response against a particular antigen or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment.

The term “tumor-associated antigen” refers to an antigen that is produced in a tumor and can be detected by the immune system to trigger an immune response. Tumor-associated antigens have been identified in many human cancers including lung, skin, hematologic, brain, liver, breast, rectal, bladder, and stomach cancers.

As used herein, “vaccine” refers to a formulation which contains an amphiphilic polyadenine nucleic acid sequence and/or polythymidine nucleic acid sequence and antigen described herein, optionally combined with an adjuvant, which is in a form that is capable of being administered to a vertebrate and which induces a protective immune response sufficient to induce immunity to prevent and/or ameliorate a disease or condition (e.g., influenza or SARS-CoV-2) and/or to reduce at least one symptom of a disease or condition (e.g., influenza or SARS-CoV-2). Typically, the vaccine comprises a conventional saline or buffered aqueous solution medium in which a composition as described herein is suspended or dissolved. In this form, a composition as described herein is used to prevent, ameliorate, or otherwise treat an infection or disease. Upon introduction into a host, the vaccine provokes an immune response including, but not limited to, the inducing a protective immune response to induce immunity to prevent and/or ameliorate a disease or condition (e.g., influenza or SARS-CoV-2) and/or to reduce at least one symptom of a disease or condition.

### DETAILED DESCRIPTION

The disclosure provides compounds including a poly-deoxyadenosine (poly-dA) nucleic acid sequence and/or a poly-deoxythymidine (poly-dT) nucleic acid sequence or a poly-deoxyguanosine (poly-dG) nucleic acid sequence and/or a poly-deoxycytosine (poly-dC) nucleic acid sequence, as well as poly-dA and/or poly-dT nucleic acid sequences and poly-dG and/or poly-dC nucleic acid sequences conjugated with an albumin-binding domain, and this disclosure provides a poly-deoxyguanosine (poly-dG) nucleic acid sequence and/or a poly-deoxycytosine (poly-dC) nucleic acid sequence, as well as poly-dG and/or poly-dC nucleic acid sequences conjugated with an albumin-binding domain, e.g., a lipid. Pharmaceutically acceptable salts thereof are also provided. Furthermore, the disclosure provides pharmaceutical compositions and kits including the poly-dA and/or poly-dT nucleic acid sequences (e.g., a poly-dA and/or poly-dT nucleic acid sequence conjugated to an albumin-binding domain, e.g., a lipid) and poly-dG and/or poly-dC nucleic acid sequences (e.g., a poly-dG and/or poly-dC nucleic acid sequence conjugated to an albumin-binding domain, e.g., a lipid) and an antigen, and pharmaceutically acceptable salts thereof. The disclosure further provides methods of inducing an immune response in a subject by administering the compounds or pharmaceutically acceptable salts thereof described herein with an antigen and the compounds including the poly-dA and/or poly-dT nucleic acid sequences and the poly-dG and/or poly-dC nucleic acid sequences, or the poly-dA and/or poly-dT nucleic acid sequences and the poly-dG and/or poly-dC nucleic acid sequences conjugated to an albumin-binding domain, e.g., a lipid.

### Polyadenosine and Polythymidine Nucleic Acid Sequences

Poly-deoxyribonucleic acids include a strand of nucleic acids that may be single or double stranded. The poly-deoxyribonucleic acid may include only one type of nucleic acid in a single strand of nucleic acids (e.g., polyadenosine (poly-dA) and polythymidine (poly-dT)). A poly-dA nucleic acid sequence may include only adenosine nucleobases. In some embodiments, the poly-dA nucleic acid sequence includes a mixture of adenosine and thymidine nucleic acid residues. For example, in some embodiments, the poly-dA nucleic acid sequence may be composed of between 100% and 51% of adenosine nucleic acid residues and between 0% and 49% thymidine nucleic acid residues. In some embodiments, the nucleic acid sequence may include alternating dA and dT residues. A poly-dT sequence may include only thymidine nucleobases. In some embodiments, the poly-dT nucleic acid sequence includes a mixture of thymidine and adenosine nucleic acid residues. For example, in some embodiments, the poly-dT nucleic acid sequence may be composed of between 100% and 51% of thymidine nucleic acid residues and between 0% and 49% adenosine nucleic acid residues.

The poly-dA single single-stranded DNA sequence may include between 75 and 100 (e.g., between 75 and 95, 75 and 90, 75 and 85, 75 and 80, 80 and 100, 85 and 100, 90 and 100, and 95 and 100) nucleotides. In some embodiments, the poly-dA single-single stranded DNA sequence includes between 75 and 150 (e.g., between 75 and 140, 75 and 130, 75 and 120, 75 and 110, 75 and 100, 75 and 95, 75 and 90, 75 and 85, 75 and 80, 80 and 150, 85 and 150, 90 and 150, 95 and 150,100 and 150, 110 and 150, 120 and 150, 130 and 150, and 140 and 150) nucleotides. In some embodiments, the poly-dA single-single stranded DNA sequence includes 75, 80, 85, 90, 95, or 100 nucleotides. In some embodiments, the poly-dA includes 50 nucleotides (dA50)

The poly-dT single single-stranded DNA sequence may include between 75 and 100 (e.g., between 75 and 95, 75 and 90, 75 and 85, 75 and 80, 80 and 100, 85 and 100, 90 and 100, and 95 and 100) nucleotides. In some embodiments, the poly-dT single-single stranded DNA sequence includes between 75 and 150 (e.g., between 75 and 140, 75 and 130, 75 and 120, 75 and 110, 75 and 100, 75 and 95, 75 and 90, 75 and 85, 75 and 80, 80 and 150, 85 and 150, 90 and 150, 95 and 150,100 and 150, 110 and 150, 120 and 150, 130 and 150, and 140 and 150) nucleotides. In some embodiments, the poly-dT single-single stranded DNA sequence includes 75, 80, 85, 90, 95, or 100 nucleotides. In some embodiments, the poly-dT includes 50 nucleotides (dT50).

The poly-dA and poly-dT double-stranded DNA sequence may include between 75 and 100 (e.g., between 75 and 95, 75 and 90, 75 and 85, 75 and 80, 80 and 100, 85 and 100, 90 and 100, and 95 and 100) nucleotides. In some embodiments, the poly-dA and poly-dT nucleic acids sequences include between 75 and 150 (e.g., between 75 and 140, 75 and 130, 75 and 120, 75 and 110, 75 and 100, 75 and 95, 75 and 90, 75 and 85, 75 and 80, 80 and 150, 85 and 150, 90 and 150, 95 and 150,100 and 150, 110 and 150, 120 and 150, 130 and 150, and 140 and 150) nucleotides. In some embodiments, the poly-dA and poly-dT include the same number of nucleotides. In some embodiments, the poly-dA and poly-dA single-singles stranded DNA sequences of the double-stranded DNA sequence each includes 75, 80, 85, 90, 95, or 100 nucleotides. In some embodiments, the poly-dA and poly-dT include 50 nucleotides each (dA50:dT50).

Reference to poly-deoxyribonucleic acids described herein, is to be understood as including pharmaceutically acceptable salts thereof.

### Polyguanosine and Polycytosine Nucleic Acid Sequences

Poly-deoxyribonucleic acids include a strand of nucleic acids that may be single or double stranded. The poly-deoxyribonucleic acid may include only one type of nucleic acid in a single strand of nucleic acids (e.g., polyguanosine (poly-dG) and polycytosine (poly-dC)). A poly-dG nucleic acid sequence may include only guanosine nucleobases. In some embodiments, the poly-dG nucleic acid sequence includes a mixture of guanosine and cytosine nucleic acid residues. For example, in some embodiments, the poly-dG nucleic acid sequence may be composed of between 100% and 51% of guanosine nucleic acid residues and between 0% and 49% cytosine nucleic acid residues. A poly-dC sequence may include only cytosine nucleobases. In some embodiments, the poly-dC nucleic acid sequence includes a mixture of cytosine and guanosine nucleic acid residues. For example, in some embodiments, the poly-dC nucleic acid sequence may be composed of between 100% and 51% of cytosine nucleic acid residues and between 0% and 49% guanosine nucleic acid residues.

The poly-dG single-stranded DNA sequence may include between 75 and 100 (e.g., between 75 and 95, 75 and 90, 75 and 85, 75 and 80, 80 and 100, 85 and 100, 90 and 100, and 95 and 100) nucleotides. In some embodiments, the poly-dG single stranded DNA sequence includes between 75 and 150 (e.g., between 75 and 140, 75 and 130, 75 and 120, 75 and 110, 75 and 100, 75 and 95, 75 and 90, 75 and 85, 75 and 80, 80 and 150, 85 and 150, 90 and 150, 95 and 150,100 and 150,110 and 150, 120 and 150, 130 and 150, and 140 and 150) nucleotides. In some embodiments, the poly-dG single stranded DNA sequence includes 75, 80, 85, 90, 95, or 100 nucleotides.

The poly-dC single-stranded DNA sequence may include between 75 and 100 (e.g., between 75 and 95, 75 and 90, 75 and 85, 75 and 80, 80 and 100, 85 and 100, 90 and 100, and 95 and 100) nucleotides. In some embodiments, the poly-dC single stranded DNA sequence includes between 75 and 150 (e.g., between 75 and 140, 75 and 130, 75 and 120, 75 and 110, 75 and 100, 75 and 95, 75 and 90, 75 and 85, 75 and 80, 80 and 150, 85 and 150, 90 and 150, 95 and 150, 100 and 150, 110 and 150, 120 and 150, 130 and 150, and 140 and 150) nucleotides. In some embodiments, the poly-dC single stranded DNA sequence includes 75, 80, 85, 90, 95, or 100 nucleotides.

The poly-dG and poly-dC double-stranded DNA sequence may include between 75 and 100 (e.g., between 75 and 95, 75 and 90, 75 and 85, 75 and 80, 80 and 100, 85 and 100, 90 and 100, and 95 and 100) nucleotides. In some embodiments, the poly-dG and poly-dC nucleic acids sequences include between 75 and 150 (e.g., between 75 and 140, 75 and 130, 75 and 120, 75 and 110, 75 and 100, 75 and 95, 75 and 90, 75 and 85, 75 and 80, 80 and 150, 85 and 150, 90 and 150, 95 and 150,100 and 150, 110 and 150, 120 and 150, 130 and 150, and 140 and 150) nucleotides. In some embodiments, the poly-dG and poly-dC include the same number of nucleotides. In some embodiments, the poly-dG and poly-dC single stranded DNA sequences of the double-stranded DNA sequence each includes 75, 80, 85, 90, 95, or 100 nucleotides. In some embodiments, the poly-dG and poly-dC strands of nucleic acids are complementary to one another

Reference to poly-deoxyribonucleic acids described herein, is to be understood as including pharmaceutically acceptable salts thereof.

### Adjuvants

### Interferon Stimulatory DNA Sequences

Interferon stimulatory DNA (ISD) is a strand of DNA that enhances expression of interferon in a cell. The ISD may originate from genome that is separate from the genome of the host cell. For example, the ISD may originate from a bacterial genome (e.g., Listeria monocytogenes). Amphiphilic ISD include a strand of nucleic acids conjugated to an albumin-binding domain, e.g., a lipid.

The disclosure provides an ISD sequence conjugated to albumin-binding domain, or a pharmaceutically acceptable salt thereof. In certain embodiments, the amphiphilic ISD is a single strand of nucleic acids conjugated to an albumin-binding domain, e.g., a lipid. In some embodiments, the amphiphilic ISD is double-stranded nucleic acid. In some embodiments, the albumin-binding domain is conjugated to the 5′ end of the ISD sequence. In other embodiments, the albumin-binding domain is conjugated to the 3′ end of the ISD sequence.

The length of the ISD sequence may include between 30 and 150 nucleotides (e.g., between 30 and 140, 30 and 130, 30 and 120, 30 and 110, 30 and 100, 30 and 90, 30 and 80, 30 and 70, 30 and 60, 30 and 50, 30 and 40, 40 and 150, 50 and 150, 60 and 150, 70 and 150, 80 and 150, 90 and 150,100 and 150, 110 and 150, 120 and 150, 130 and 150, and 140 and 150 nucleotides). For example, the length of the ISD sequence may be between 30 and 100 nucleotides (e.g., between 30 and 90, 30 and 80, 30 and 70, 30 and 60, 30 and 50, 30 and 40, 40 and 100, 50 and 100, 60 and 100, 70 and 100, 80 and 100, and 90 and 100 nucleotides). In some embodiments, the length of the ISD sequence may be between 50 and 100 nucleotides (e.g., between 50 and 90, 50 and 80, 50 and 70, 50 and 60, 60 and 100, 70 and 100, 80 and 100, and 90 and 100 nucleotides). In some embodiments, the length of the ISD sequence may be between 30 and 50 nucleotides (e.g., between 30 and 45, 30 and 40, 30 and 35, 35 and 50, 40 and 50, and 45 and 50 nucleotides). In some embodiments, the length of the ISD sequence may be 30, 35, 40, 45, 50, 55, 60, 65, 70, 75, 80, 85, 90, 95, 100, 105, 110, 115, 120, 125, 130, 135, 140, 145, or 150 nucleotides. In some embodiments, the length of the ISD sequence may be 30, 35, 40, 45, 50, 55, 60, 65, 70, 75, 80, 85, 90, 95, or 100 nucleotides.

In some embodiments, between 50% and 100% (e.g., between 50% and 90%, 50% and 80%, 50% and 70%, 50% and 60%, 60% and 100%, 70% and 100%, 80% and 100%, or 90% and 100%) of the internucleotide groups connecting the nucleotides in the ISD sequence are phosphorothioate linkages. In some embodiments, between 1 and 10 (e.g., 1, 2, 3, 4, 5, 6, 7, 8, 9, or 10) of the internucleotide groups connecting the nucleotides in the ISD sequence are phosphodiester linkages and the remaining internucleotide groups connecting the nucleotides of the ISD sequence are phosphorothioate linkages. For example, the between 1 and 5 linkages (e.g., 1, 2, 3, 4, or 5) linkages at the 5′ and/or 3′ end of the ISD sequence are phosphodiester linkages, and all the remaining linkages are phosphorothioate linkages.

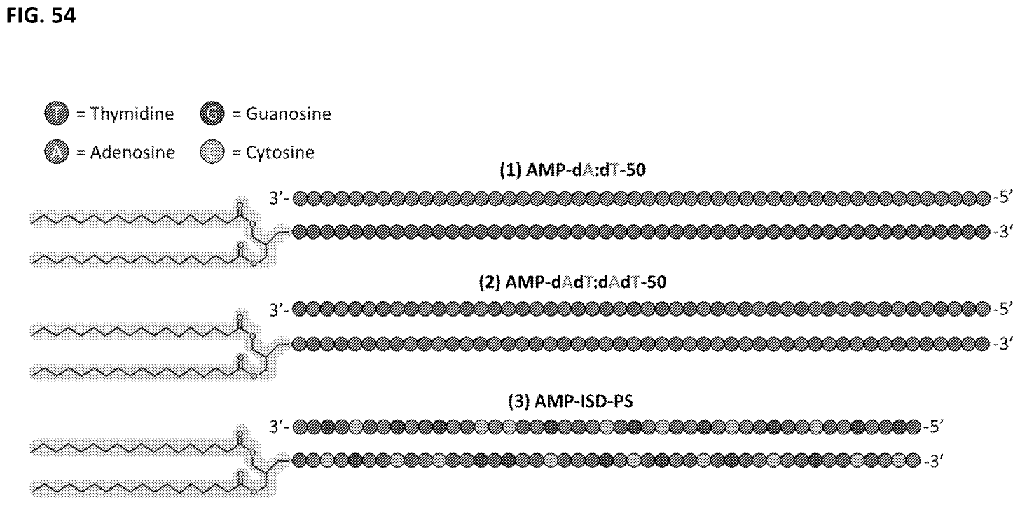

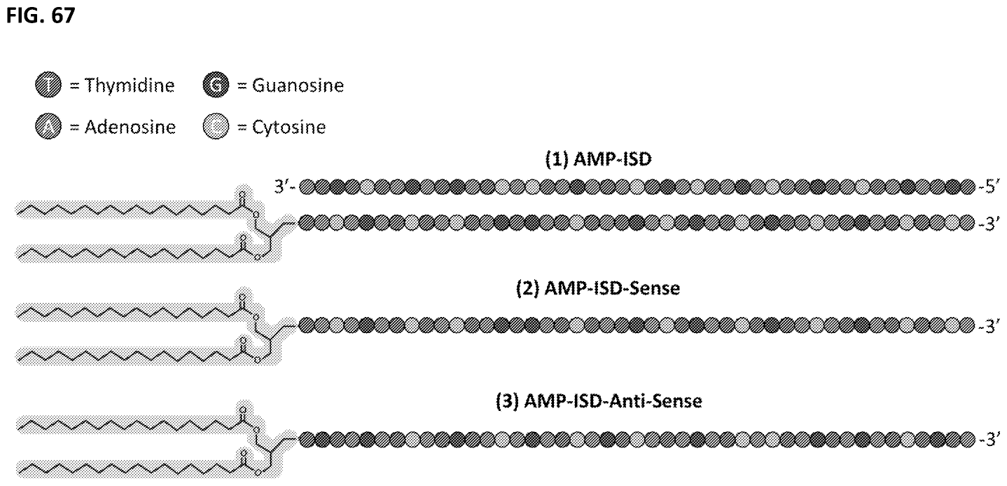

The ISD may be either double-stranded or single-stranded. The double-stranded nucleic acid may include a sense strand and antisense strand. For example, the ISD may include a sense strand having the nucleic acid sequence of TACAGATCTACTAGTGATCTATGACTGATCTGTACATGATCTACA (SEQ ID NO: 23) and an antisense strand having the nucleic acids sequence of ATGTCTAGATGATCACTAGATACTGACTAGACATGTACTAGATGT (SEQ ID NO: 24) as is shown in FIG. 54 and FIG. 67.

Reference to adjuvants described herein, is to be understood as including pharmaceutically acceptable salts thereof.

### Herpes Simplex Virus

The disclosure provides a Herpes Simplex Virus (HSV) sequence conjugated to albumin-binding domain, or a pharmaceutically acceptable salt thereof. In certain embodiments, the amphiphilic HSV is a single strand of nucleic acids conjugated to an albumin-binding domain, e.g., a lipid. In some embodiments, the amphiphilic HSV is double-stranded nucleic acid. In some embodiments, the albumin-binding domain is conjugated to the 5′ end of the HSV sequence. In other embodiments, the albumin-binding domain is conjugated to the 3′ end of the HSV sequence.

The length of the HSV sequence may include between 30 and 150 nucleotides (e.g., between 30 and 140, 30 and 130, 30 and 120, 30 and 110, 30 and 100, 30 and 90, 30 and 80, 30 and 70, 30 and 60, 30 and 50, 30 and 40, 40 and 150, 50 and 150, 60 and 150, 70 and 150, 80 and 150, 90 and 150,100 and 150, 110 and 150, 120 and 150, 130 and 150, and 140 and 150 nucleotides). For example, the length of the HSV sequence may be between 30 and 100 nucleotides (e.g., between 30 and 90, 30 and 80, 30 and 70, 30 and 60, 30 and 50, 30 and 40, 40 and 100, 50 and 100, 60 and 100, 70 and 100, 80 and 100, and 90 and 100 nucleotides). In some embodiments, the length of the HSV sequence may be between 50 and 100 nucleotides (e.g., between 50 and 90, 50 and 80, 50 and 70, 50 and 60, 60 and 100, 70 and 100, 80 and 100, and 90 and 100 nucleotides). In some embodiments, the length of the HSV sequence may be between 30 and 50 nucleotides (e.g., between 30 and 45, 30 and 40, 30 and 35, 35 and 50, 40 and 50, and 45 and 50 nucleotides). In some embodiments, the length of the HSV sequence may be 30, 35, 40, 45, 50, 55, 60, 65, 70, 75, 80, 85, 90, 95, 100, 105, 110, 115, 120, 125, 130, 135, 140, 145, or 150 nucleotides. In some embodiments, the length of the HSV sequence may be 30, 35, 40, 45, 50, 55, 60, 65, 70, 75, 80, 85, 90, 95, or 100 nucleotides. In some embodiments, the length of the HSV sequence is about 60 nucleotides.

In some embodiments, between 50% and 100% (e.g., between 50% and 90%, 50% and 80%, 50% and 70%, 50% and 60%, 60% and 100%, 70% and 100%, 80% and 100%, or 90% and 100%) of the internucleotide groups connecting the nucleotides in the HSV sequence are phosphorothioate linkages. In some embodiments, between 1 and 10 (e.g., 1, 2, 3, 4, 5, 6, 7, 8, 9, or 10) of the internucleotide groups connecting the nucleotides in the HSV sequence are phosphodiester linkages and the remaining internucleotide groups connecting the nucleotides of the HSV sequence are phosphorothioate linkages. For example, the between 1 and 5 linkages (e.g., 1, 2, 3, 4, or 5) linkages at the 5′ and/or 3′ end of the HSV sequence are phosphodiester linkages, and all the remaining linkages are phosphorothioate linkages.

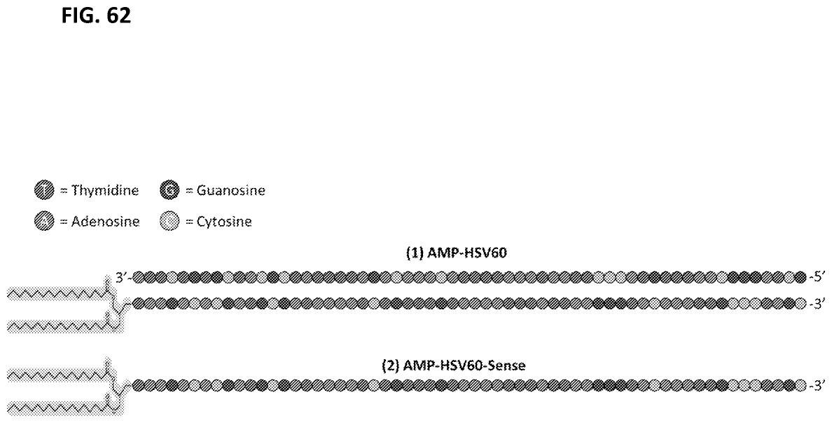

The HSV may be either double-stranded or single-stranded. The double-stranded nucleic acid may include a sense strand and antisense strand. For example, the HSV may include a sense strand and an antisense strand such as HSV sequences shown in FIG. 62. In some embodiments, the HSV sequence may be an HSV-60 sequence. In some embodiments, the HSV-60 sequence sense strand has the following sequence (5′ to 3′) TAAGACACGATGCGATAAAATCTGTTTGTAAAATTTATTAAGGGTACAAATTGCCCTAGC (SEQ ID NO: 34). In some embodiments, the HSV-60 sequence anti-sense strand has the following sequence (5′ to 3′) GCTAGGGCAATTTGTACCCTTAATAAATTTTACAAACAGATTTTATCGCATCGTGTCTTA (SEQ ID NO: 35) as is shown in FIG. 62, where the 5′ end of the sense strand is bonded to a diacyl lipid.

### Amphiphilic Poly-Deoxyribonucleic Acids

Amphiphilic poly-deoxyribonucleic acids include a strand of nucleic acids conjugated to an albumin-binding domain, e.g., a lipid. In certain embodiments, the amphiphilic poly-deoxyribonucleic acid is a poly-deoxyadenosine (AMP-dA) strand of nucleic acids conjugated to an albumin-binding domain, e.g., a lipid. In certain embodiments, the amphiphilic poly-deoxyribonucleic acid is a poly-deoxythymidine (AMP-dT) strand of nucleic acids conjugated to an albumin-binding domain, e.g., a lipid. In certain embodiments, the amphiphilic poly-deoxyribonucleic acid is a poly-deoxyguanosine (AMP-dG) strand of nucleic acids conjugated to an albumin-binding domain, e.g., a lipid. In certain embodiments, the amphiphilic poly-deoxyribonucleic acid is a poly-deoxycytosine (AMP-dC) strand of nucleic acids conjugated to an albumin-binding domain, e.g., a lipid.

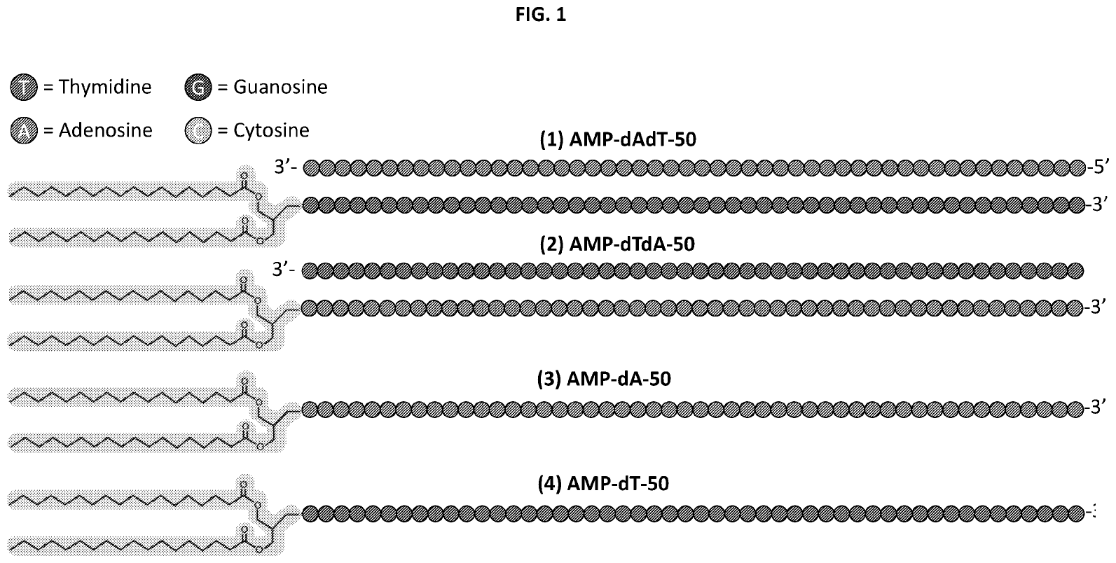

In some embodiments, the compounds described herein include both a dA and a dT nucleic acid sequence, or a dG and a dC, and an albumin-binding domain (e.g., AMP-dA:dT, AMP-dT:dA, AMP-dG:dC, and AMP-dC:dG). The dA nucleic acid sequence and the dT nucleic acid sequence may hybridize to form a double-stranded DNA sequence (e.g., dA:dT and dT:dA). Likewise, the dG nucleic acid sequence and the dC nucleic acid sequence may hybridize to form a double-stranded DNA sequence (e.g., dG:dC and dC:dG). Examples of the poly-dA and/or poly-dT compounds of the disclosure are shown in FIG. 1 and FIG. 54. In some embodiments, the compounds describe herein include a mixture of both dA and dT nucleic acid residues or a mixture of dG and dC nucleic acid residues. For example, the compound may include alternating dA and dT nucleic acid residues.

The length of the poly-dA nucleic acid sequence may include between 30 and 150 nucleotides (e.g., between 30 and 140, 30 and 130, 30 and 120, 30 and 110, 30 and 100, 30 and 90, 30 and 80, 30 and 70, 30 and 60, 30 and 50, 30 and 40, 40 and 150, 50 and 150, 60 and 150, 70 and 150, 80 and 150, 90 and 150, 100 and 150, 110 and 150, 120 and 150, 130 and 150, and 140 and 150 nucleotides). For example, the length of the poly-dA nucleic acid sequence may be between 30 and 100 nucleotides (e.g., between 30 and 90, 30 and 80, 30 and 70, 30 and 60, 30 and 50, 30 and 40, 40 and 100, 50 and 100, 60 and 100, 70 and 100, 80 and 100, and 90 and 100 nucleotides). In some embodiments, the length of the poly-dA nucleic acid sequence may be between 50 and 100 nucleotides (e.g., between 50 and 90, 50 and 80, 50 and 70, 50 and 60, 60 and 100, 70 and 100, 80 and 100, and 90 and 100 nucleotides). In some embodiments, the length of the poly-dA nucleic acid sequence may be between 30 and 50 nucleotides (e.g., between 30 and 45, 30 and 40, 30 and 35, 35 and 50, 40 and 50, and 45 and 50 nucleotides). In some embodiments, the length of the poly-dA nucleic acid sequence may be 30, 35, 40, 45, 50, 55, 60, 65, 70, 75, 80, 85, 90, 95, 100, 105, 110, 115, 120, 125, 130, 135, 140, 145, or 150 nucleotides. In some embodiments, the length of the poly-dA nucleic acid sequence may be 30, 35, 40, 45, 50, 55, 60, 65, 70, 75, 80, 85, 90, 95, or 100 nucleotides. Furthermore, the disclosure provides a poly-dA single single-stranded DNA sequence including between 75 and 100 (e.g., between 75 and 95, 75 and 90, 75 and 85, 75 and 80, 80 and 100, 85 and 100, 90 and 100, and 95 and 100) nucleotides.

The length of the poly-dT nucleic acid sequence may include between 30 and 150 nucleotides (e.g., between 30 and 140, 30 and 130, 30 and 120, 30 and 110, 30 and 100, 30 and 90, 30 and 80, 30 and 70, 30 and 60, 30 and 50, 30 and 40, 40 and 150, 50 and 150, 60 and 150, 70 and 150, 80 and 150, 90 and 150, 100 and 150, 110 and 150, 120 and 150, 130 and 150, and 140 and 150 nucleotides). For example, the length of the poly-dT nucleic acid sequence may be between 30 and 100 nucleotides (e.g., between 30 and 90, 30 and 80, 30 and 70, 30 and 60, 30 and 50, 30 and 40, 40 and 100, 50 and 100, 60 and 100, 70 and 100, 80 and 100, and 90 and 100 nucleotides). In some embodiments, the length of the poly-dT nucleic acid sequence may be between 50 and 100 nucleotides (e.g., between 50 and 90, 50 and 80, 50 and 70, 50 and 60, 60 and 100, 70 and 100, 80 and 100, and 90 and 100 nucleotides). In some embodiments, the length of the poly-dT nucleic acid sequence may be between 30 and 50 nucleotides (e.g., between 30 and 45, 30 and 40, 30 and 35, 35 and 50, 40 and 50, and 45 and 50 nucleotides). In some embodiments, the length of the poly-dT nucleic acid sequence may be 30, 35, 40, 45, 50, 55, 60, 65, 70, 75, 80, 85, 90, 95, 100, 105, 110, 115, 120, 125, 130, 135, 140, 145, or 150 nucleotides. In some embodiments, the length of the poly-dT nucleic acid sequence may be 30, 35, 40, 45, 50, 55, 60, 65, 70, 75, 80, 85, 90, 95, or 100 nucleotides. Furthermore, the disclosure provides a poly-dT single single-stranded DNA sequence including between 75 and 100 (e.g., between 75 and 95, 75 and 90, 75 and 85, 75 and 80, 80 and 100, 85 and 100, 90 and 100, and 95 and 100) nucleotides.

The length of both the poly-dA nucleic acid sequence and the poly-dT nucleic acid sequence may both include between 30 and 150 nucleotides (e.g., between 30 and 140, 30 and 130, 30 and 120, 30 and 110, 30 and 100, 30 and 90, 30 and 80, 30 and 70, 30 and 60, 30 and 50, 30 and 40, 40 and 150, 50 and 150, 60 and 150, 70 and 150, 80 and 150, 90 and 150, 100 and 150, 110 and 150, 120 and 150, 130 and 150, and 140 and 150 nucleotides). For example, the length of both the poly-dA nucleic acid sequence and the poly-dT nucleic acid sequence may be between 30 and 100 nucleotides (e.g., between 30 and 90, 30 and 80, 30 and 70, 30 and 60, 30 and 50, 30 and 40, 40 and 100, 50 and 100, 60 and 100, 70 and 100, 80 and 100, and 90 and 100 nucleotides). In some embodiments, the length of both the poly-dA nucleic acid sequence and the poly-dT nucleic acid sequence may be between 50 and 100 nucleotides (e.g., between 50 and 90, 50 and 80, 50 and 70, 50 and 60, 60 and 100, 70 and 100, 80 and 100, and 90 and 100 nucleotides). In some embodiments, the length of both the poly-dA nucleic acid sequence and the poly-dT nucleic acid sequence may be between 30 and 50 nucleotides (e.g., between 30 and 45, 30 and 40, 30 and 35, 35 and 50, 40 and 50, and 45 and 50 nucleotides). In some embodiments, the length of both the poly-dA nucleic acid sequence and the poly-dT nucleic acid sequence may be 30, 40, 50, 75, or 100 nucleotides. In some embodiments, the poly-dA nucleic acid sequence and poly-dT nucleic acid sequence nucleotides the same number of nucleotides. Furthermore, the disclosure provides a poly-dA and poly-dT double-stranded DNA sequence including between 30 and 100 (e.g., between 30 and 90, 30 and 80, 30 and 70, 30 and 60, 30 and 50, 30 and 40, 40 and 100, 50 and 100, 60 and 100, 70 and 100, 80 and 100, and 90 and 100) nucleotides. In some embodiments, the poly-dA and poly-dT include the same number of nucleotides.

The length of the poly-dG nucleic acid sequence may include between 30 and 150 nucleotides (e.g., between 30 and 140, 30 and 130, 30 and 120, 30 and 110, 30 and 100, 30 and 90, 30 and 80, 30 and 70, 30 and 60, 30 and 50, 30 and 40, 40 and 150, 50 and 150, 60 and 150, 70 and 150, 80 and 150, 90 and 150, 100 and 150, 110 and 150, 120 and 150, 130 and 150, and 140 and 150 nucleotides). For example, the length of the poly-dG nucleic acid sequence may be between 30 and 100 nucleotides (e.g., between 30 and 90, 30 and 80, 30 and 70, 30 and 60, 30 and 50, 30 and 40, 40 and 100, 50 and 100, 60 and 100, 70 and 100, 80 and 100, and 90 and 100 nucleotides). In some embodiments, the length of the poly-dG nucleic acid sequence may be between 50 and 100 nucleotides (e.g., between 50 and 90, 50 and 80, 50 and 70, 50 and 60, 60 and 100, 70 and 100, 80 and 100, and 90 and 100 nucleotides). In some embodiments, the length of the poly-dG nucleic acid sequence may be between 30 and 50 nucleotides (e.g., between 30 and 45, 30 and 40, 30 and 35, 35 and 50, 40 and 50, and 45 and 50 nucleotides). In some embodiments, the length of the poly-dG nucleic acid sequence may be 30, 35, 40, 45, 50, 55, 60, 65, 70, 75, 80, 85, 90, 95, 100, 105, 110, 115, 120, 125, 130, 135, 140, 145, or 150 nucleotides. In some embodiments, the length of the poly-dG nucleic acid sequence may be 30, 35, 40, 45, 50, 55, 60, 65, 70, 75, 80, 85, 90, 95, or 100 nucleotides. Furthermore, the disclosure provides a poly-dG single single-stranded DNA sequence including between 75 and 100 (e.g., between 75 and 95, 75 and 90, 75 and 85, 75 and 80, 80 and 100, 85 and 100, 90 and 100, and 95 and 100) nucleotides.

The length of the poly-dC nucleic acid sequence may include between 30 and 150 nucleotides (e.g., between 30 and 140, 30 and 130, 30 and 120, 30 and 110, 30 and 100, 30 and 90, 30 and 80, 30 and 70, 30 and 60, 30 and 50, 30 and 40, 40 and 150, 50 and 150, 60 and 150, 70 and 150, 80 and 150, 90 and 150, 100 and 150, 110 and 150, 120 and 150, 130 and 150, and 140 and 150 nucleotides). For example, the length of the poly-dC nucleic acid sequence may be between 30 and 100 nucleotides (e.g., between 30 and 90, 30 and 80, 30 and 70, 30 and 60, 30 and 50, 30 and 40, 40 and 100, 50 and 100, 60 and 100, 70 and 100, 80 and 100, and 90 and 100 nucleotides). In some embodiments, the length of the poly-dC nucleic acid sequence may be between 50 and 100 nucleotides (e.g., between 50 and 90, 50 and 80, 50 and 70, 50 and 60, 60 and 100, 70 and 100, 80 and 100, and 90 and 100 nucleotides). In some embodiments, the length of the poly-dC nucleic acid sequence may be between 30 and 50 nucleotides (e.g., between 30 and 45, 30 and 40, 30 and 35, 35 and 50, 40 and 50, and 45 and 50 nucleotides). In some embodiments, the length of the poly-dC nucleic acid sequence may be 30, 35, 40, 45, 50, 55, 60, 65, 70, 75, 80, 85, 90, 95, 100, 105, 110, 115, 120, 125, 130, 135, 140, 145, or 150 nucleotides. In some embodiments, the length of the poly-dC nucleic acid sequence may be 30, 35, 40, 45, 50, 55, 60, 65, 70, 75, 80, 85, 90, 95, or 100 nucleotides. Furthermore, the disclosure provides a poly-dC single single-stranded DNA sequence including between 75 and 100 (e.g., between 75 and 95, 75 and 90, 75 and 85, 75 and 80, 80 and 100, 85 and 100, 90 and 100, and 95 and 100) nucleotides.

The length of both the poly-dG nucleic acid sequence and the poly-dC nucleic acid sequence may both include between 30 and 150 nucleotides (e.g., between 30 and 140, 30 and 130, 30 and 120, 30 and 110, 30 and 100, 30 and 90, 30 and 80, 30 and 70, 30 and 60, 30 and 50, 30 and 40, 40 and 150, 50 and 150, 60 and 150, 70 and 150, 80 and 150, 90 and 150,100 and 150,110 and 150,120 and 150, 130 and 150, and 140 and 150 nucleotides). For example, the length of both the poly-dG nucleic acid sequence and the poly-dC nucleic acid sequence may be between 30 and 100 nucleotides (e.g., between 30 and 90, 30 and 80, 30 and 70, 30 and 60, 30 and 50, 30 and 40, 40 and 100, 50 and 100, 60 and 100, 70 and 100, 80 and 100, and 90 and 100 nucleotides). In some embodiments, the length of both the poly-dG nucleic acid sequence and the poly-dC nucleic acid sequence may be between 50 and 100 nucleotides (e.g., between 50 and 90, 50 and 80, 50 and 70, 50 and 60, 60 and 100, 70 and 100, 80 and 100, and 90 and 100 nucleotides). In some embodiments, the length of both the poly-dG nucleic acid sequence and the poly-dC nucleic acid sequence may be between 30 and 50 nucleotides (e.g., between 30 and 45, 30 and 40, 30 and 35, 35 and 50, 40 and 50, and 45 and 50 nucleotides). In some embodiments, the length of both the poly-dG nucleic acid sequence and the poly-dC nucleic acid sequence may be 30, 40, 50, 75, or 100 nucleotides. In some embodiments, the poly-dG nucleic acid sequence and poly-dC nucleic acid sequence nucleotides the same number of nucleotides. Furthermore, the disclosure provides a poly-dG and poly-dC double-stranded DNA sequence including between 30 and 100 (e.g., between 30 and 90, 30 and 80, 30 and 70, 30 and 60, 30 and 50, 30 and 40, 40 and 100, 50 and 100, 60 and 100, 70 and 100, 80 and 100, and 90 and 100) nucleotides. In some embodiments, the poly-dG and poly-dC include the same number of nucleotides.

The length of the alternating poly-dA and poly-dT nucleic acid sequence may include between 30 and 150 nucleotides (e.g., between 30 and 140, 30 and 130, 30 and 120, 30 and 110, 30 and 100, 30 and 90, 30 and 80, 30 and 70, 30 and 60, 30 and 50, 30 and 40, 40 and 150, 50 and 150, 60 and 150, 70 and 150, 80 and 150, 90 and 150, 100 and 150, 110 and 150, 120 and 150, 130 and 150, and 140 and 150 nucleotides). For example, the length of the poly-dA nucleic acid sequence may be between 30 and 100 nucleotides (e.g., between 30 and 90, 30 and 80, 30 and 70, 30 and 60, 30 and 50, 30 and 40, 40 and 100, 50 and 100, 60 and 100, 70 and 100, 80 and 100, and 90 and 100 nucleotides). In some embodiments, the length of the alternating poly-dA and poly-dT nucleic acid sequence may be between 50 and 100 nucleotides (e.g., between 50 and 90, 50 and 80, 50 and 70, 50 and 60, 60 and 100, 70 and 100, 80 and 100, and 90 and 100 nucleotides). In some embodiments, the length of the poly-dA nucleic acid sequence may be between 30 and 50 nucleotides (e.g., between 30 and 45, 30 and 40, 30 and 35, 35 and 50, 40 and 50, and 45 and 50 nucleotides). In some embodiments, the length of the poly-dA nucleic acid sequence may be 30, 35, 40, 45, 50, 55, 60, 65, 70, 75, 80, 85, 90, 95, 100, 105, 110, 115, 120, 125, 130, 135, 140, 145, or 150 nucleotides. In some embodiments, the length of the alternating poly-dA and poly-dT nucleic acid sequence may be 30, 35, 40, 45, 50, 55, 60, 65, 70, 75, 80, 85, 90, 95, or 100 nucleotides. Furthermore, the disclosure provides an alternating poly-dA and poly-dT single single-stranded DNA sequence including between 75 and 100 (e.g., between 75 and 95, 75 and 90, 75 and 85, 75 and 80, 80 and 100, 85 and 100, 90 and 100, and 95 and 100) nucleotides.

In some embodiments, between 50% and 100% (e.g., between 50% and 90%, 50% and 80%, 50% and 70%, 50% and 60%, 60% and 100%, 70% and 100%, 80% and 100%, or 90% and 100%) of the internucleotide groups connecting the nucleotides in the poly-dA nucleic acid sequence and/or poly-dT nucleic acid sequence are phosphorothioate linkages. In some embodiments, between 1 and 10 (e.g., 1, 2, 3, 4, 5, 6, 7, 8, 9, or 10) of the internucleotide groups connecting the nucleotides in the poly-dA nucleic acid sequence and/or poly-dT nucleic acid sequence are phosphodiester linkages and the remaining internucleotide groups connecting the nucleotides of the poly-dA nucleic acid sequence and/or poly-dT nucleic acid sequence are phosphorothioate linkages. For example, the between 1 and 5 linkages (e.g., 1, 2, 3, 4, or 5) linkages at the 5′ and/or 3′ end of the poly-dA and/or poly-dT nucleic acid sequences are phosphodiester linkages, and all the remaining linkages are phosphorothioate linkages. In some embodiments, all internucleotide groups connecting the nucleotides in the poly-dA nucleic acid sequence and/or poly-dT nucleic acid sequence are phosphorothioate linkages.

In some embodiments, between 50% and 100% (e.g., between 50% and 90%, 50% and 80%, 50% and 70%, 50% and 60%, 60% and 100%, 70% and 100%, 80% and 100%, or 90% and 100%) of the internucleotide groups connecting the nucleotides in the poly-dG nucleic acid sequence and/or poly-dC nucleic acid sequence are phosphorothioate linkages. In some embodiments, between 1 and 10 (e.g., 1, 2, 3, 4, 5, 6, 7, 8, 9, or 10) of the internucleotide groups connecting the nucleotides in the poly-dG nucleic acid sequence and/or poly-dC nucleic acid sequence are phosphodiester linkages and the remaining internucleotide groups connecting the nucleotides of the poly-dG nucleic acid sequence and/or poly-dC nucleic acid sequence are phosphorothioate linkages. For example, the between 1 and 5 linkages (e.g., 1, 2, 3, 4, or 5) linkages at the 5′ and/or 3′ end of the poly-dG and/or poly-dC nucleic acid sequences are phosphodiester linkages, and all the remaining linkages are phosphorothioate linkages. In some embodiments, all internucleotide groups connecting the nucleotides in the poly-dG nucleic acid sequence and/or poly-dC nucleic acid sequence are phosphorothioate linkages.

Reference to poly-deoxyribonucleic acids described herein, as well as amphiphiles including the poly-deoxyribonucleic acids, is to be understood as including pharmaceutically acceptable salts thereof.

### Lipids

The compounds described herein include poly-deoxyribonucleotide sequences, including poly-dA, poly-dT, poly-dG, and poly-dC, an ISD, or immunostimulatory HSV sequence and a lipid. In some embodiments, the lipid is bonded to the 5′ end of the poly-dA nucleic acid sequence. In some embodiments, the lipid is bonded to the 5′ end of the poly-dT nucleic acid sequence. In some embodiments, the lipid is bonded to the 5′ end of the poly-dG nucleic acid sequence. In some embodiments, the lipid is bonded to the 5′ end of the poly-dC nucleic acid sequence. In some embodiments, the lipid is bonded to the 5′ end of the ISD sequence. In some embodiments, the lipid is bonded to the 5′ end of the immunostimulatory HSV sequence. The lipid can be linear, branched, or cyclic.

Examples of preferred lipids include, but are not limited to, fatty acids with aliphatic tails of 3-30 carbons including, but not limited to, linear unsaturated and saturated fatty acids, branched saturated and unsaturated fatty acids, and fatty acids derivatives, such as fatty acid esters, fatty acid amides, and fatty acid thioesters, diacyl lipids, cholesterol, cholesterol derivatives, and steroid acids such as bile acids, Lipid A or combinations thereof.

In certain embodiments, the lipid is a diacyl lipid or two-tailed lipid. In some embodiments, the tails in the diacyl lipid contain from about 12 to about 30 carbons (e.g., 13 14, 15, 16, 17, 18, 19, 20, 21, 22, 23, 24, 25, 26, 27, 28, or 29). In some embodiments the tails in the diacyl lipid contain about 14 to about 25 carbons (e.g., 15, 16, 17, 18, 19, 20, 21, 22, 23, or 24). In some embodiments, the tails of the diacyl lipid contain from about 16 to about 20 carbons (e.g., 17, 18, or 19). In some embodiments, the diacyl lipid comprises 12, 13, 14, 15, 16, 17, 18, 19, 20, 21, 22, 23, 24, 25, 26, 27, 28, 29 or 30 carbons.

The carbon tails of the diacyl lipid can be saturated, unsaturated, or combinations thereof. The tails can be coupled to the head group via ester bond linkages, amide bond linkages, thioester bond linkages, or combinations thereof. In a particular embodiment, the diacyl lipids are phosphate lipids, glycolipids, sphingolipids, or combinations thereof.

In some embodiments, the lipid is 1,2-distearoy-sn-gycer-3-phosphioethanolarine (DSPE).

In some embodiments, the poly-dA nucleic acid sequence, the poly-dT nucleic acid sequence, the poly-dG, the poly-dC, the ISD, or immunostimulatory HSV sequence is bonded or linked by a linker to the following lipid:

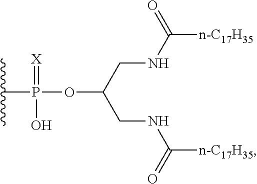

or a salt thereof, wherein X is O or S. The poly-dA nucleic acid sequence, poly-dT nucleic acid sequence, poly-dG nucleic acid sequence, the poly-dC nucleic acid sequence, ISD, or immunostimulatory HSV sequence may be directly bonded to the lipid. Alternatively, the poly-dA nucleic acid sequence, poly-dT nucleic acid sequence, poly-dG nucleic acid sequence, poly-dC nucleic acid sequence, ISD, or immunostimulatory HSV sequence may be linked to the lipid through a linker.

Reference to lipids herein, as well as amphiphiles including the lipid, is to be understood as including pharmaceutically acceptable salts thereof.

### Linkers

In some embodiments, the compound includes a poly-dA, poly-dT, poly-dG, poly-dC, ISD, or immunostimulatory HSV sequence linked to an albumin-binding domain, e.g., a lipid, by a linker. The linker may be a hydrophilic polymer, a string of hydrophilic amino acids, a polysaccharide, and an oligonucleotide, or a combination thereof. The linker may reduce or prevent the ability of the albumin-binding domain to insert into the plasma membrane of cells, such as cells in the tissue adjacent to the injection site. The linker can also reduce or prevent the ability of the amphiphilic poly-dA, amphiphilic poly-dT, amphiphilic poly-dG, or amphiphilic poly-dC from non-specifically associating with extracellular matrix proteins at the site of administration. For the amphiphilic poly-dA, the amphiphilic poly-dT, the amphiphilic poly-dG, or the amphiphilic poly-dC to be trafficked efficiently to the lymph node, it should remain soluble. A polar block linker may be included between the poly-dA, poly-dT, poly-dG, or poly-dC and the albumin-binding domain to which it is conjugated to increase solubility of the amphiphilic poly-dA, amphiphilic poly-dT, amphiphilic poly-dG, or amphiphilic poly-dC.

The length and composition of the linker can be adjusted based on the albumin-binding domain and poly-dA, poly-dT, poly-dG, poly-dC, ISD, or HSV sequence selected. For example, in certain embodiments, the polynucleotide itself may be polar enough to ensure solubility; for example, polynucleotides that are 10, 15, 20 or more nucleotides in length. Therefore, in some embodiments, no additional linker is required. However, in certain cases, it can be desirable to include a linker that mimics the effect of a polar oligonucleotide. A linker can be used as part of any of albumin-binding domain conjugates described herein, for example, lipid-oligonucleotide conjugates and lipid-peptide conjugates, which reduce cell membrane insertion/preferential portioning onto albumin.

Suitable linkers include, but are not limited to, oligonucleotides such as those discussed above, including a string of nucleic acids, a hydrophilic polymer including but not limited to poly(ethylene glycol) (MW: 500 Da to 20,000 Da), polyacrylamide (MW: 500 Da to 20,000 Da), polyacrylic acid; a string of hydrophilic amino acids such as serine, threonine, cysteine, tyrosine, asparagine, glutamine, aspartic acid, glutamic acid, lysine, arginine, histidine, or combinations thereof; polysaccharides, including but not limited to, dextran (MW: 1,000 Da to 2,000,000 Da), or combinations thereof. The hydrophobic albumin-binding domain and the linker/CpG ODN are covalently linked. The covalent bond may be a non-cleavable linkage or a cleavable linkage. The non-cleavable linkage can include an amide bond or phosphate bond, and the cleavable linkage can include a disulfide bond, acid-cleavable linkage, ester bond, anhydride bond, biodegradable bond, or enzyme-cleavable linkage.

In some embodiments, the linker is one or more ethylene glycol (EG) units, more preferably two or more EG units (i.e., polyethylene glycol (PEG)). For example, in some embodiments, compound includes a poly-dA, a poly-dT, a poly-dG, or a poly-dC and a hydrophobic albumin-binding domain linked by a polyethylene glycol (PEG) molecule or a derivative or analog thereof.

In some embodiments, compounds described herein contain a poly-dA, poly-dT, poly-dG, poly-dC, ISD, or immunostimulatory HSV sequence linked to PEG which is in turn linked to a hydrophobic albumin-binding domain, e.g., a lipid. The precise number of PEG units depends on the albumin-binding domain and the cargo, however, typically, a linker can have between about 1 and about 100, between about 20 and about 80, between about 30 and about 70, or between about 40 and about 60 PEG units.

In some embodiments, the number of PEG units is between 24 and 50 units (e.g., between 24 and 45, 24 and 40, 24 and 35, 24 and 30, 30 and 50, 35 and 50, 40 and 50, and 45 and 50 units). In some embodiments, the linker has between about 45 and 55 PEG, units. For example, in some embodiments, the linker has 48 PEG units. In some embodiments, the linker includes a PEG24-amido-PEG24 linker.

As discussed above, in some embodiments, the linker is an oligonucleotide which includes a string of nucleic acids. In some embodiments, the compounds described herein include a poly-dA, poly-dT, poly-dG, poly-dC, ISD, or immunostimulatory HSV sequence linked to a string of nucleic acids, which is in turn linked to a hydrophobic albumin-binding domain, e.g., a lipid. The linker can be any sequence, for example, the sequence of the oligonucleotide can be a random sequence, or a sequence specifically chosen for its molecular or biochemical properties (e.g., highly polar). In some embodiments, the linker includes 20 one or more series of consecutive adenine (A), cytosine (C), guanine (G), thymine (T), uracil (U), or analog thereof. In some embodiments, the linker consists of a series of consecutive adenine (A), cytosine (C), guanine (G), thymine (T), uracil (U), or analog thereof.

In some embodiments, the string of nucleic acids includes between 1 and 50 nucleic acid residues. In some embodiments, the string of nucleic acids includes between 5 and 30 nucleic acid residues. In some embodiments, the linker includes one or more guanines, for example between 1-10 guanines.

In some embodiments, the linker is an oligonucleotide that includes a string of amino acids. In some embodiments, the amphiphilic poly-dA, amphiphilic poly-dT, amphiphilic poly-dG, or amphiphilic poly-dC include a poly-dA, poly-dT, poly-dG, or poly-dC linked to string of amino acids, which is in turn linked to a hydrophobic albumin-binding domain, e.g., a lipid. The linker can have any amino acid sequence, for example, the sequence of the oligonucleotide can be a random sequence, or a sequence chosen for its molecular or biochemical properties (e.g., high flexibility). In some embodiments, the linker includes a series of glycine residue to form a polyglycine linker. In some embodiments, the linker includes an amino acid sequence of (Gly)ₙ, wherein n may be between 2 and 20 residues. Examples of polyglycine linkers include but are not limited to GGG, GGGA (SEQ ID NO:1), GGGG (SEQ ID NO:2), GGGAG (SEQ ID NO:3), GGGAGG (SEQ ID NO:4), GGGAGGG (SEQ ID NO:5), GGAG (SEQ ID NO:6), GGSG (SEQ ID NO:7), AGGG (SEQ ID NO:8), SGGG (SEQ ID NO:9), GGAGGA (SEQ ID NO:10), GGSGGS (SEQ ID NO:11), GGAGGAGGA (SEQ ID NO:12), GGSGGSGGS (SEQ ID NO:13), GGAGGAGGAGGA (SEQ ID NO:14), GGSGGSGGSGGS (SEQ ID NO:15), GGAGGGAG (SEQ ID NO:16), GGSGGGSG (SEQ ID NO:17), GGAGGGAGGGAG (SEQ ID NO:18), GGSGGGSGGGSG (SEQ ID NO:19), GGGGAGGGGAGGGGA (SEQ ID NO:20), and GGGGSGGGGSGGGGS (SEQ ID NO:21).

### Antigens

In some embodiments, the poly-dA and/or poly-dT conjugated to an albumin-binding domain, the poly-dG and/or poly-dC conjugated to an albumin-binding domain, or ISD or immunostimulatory HSV sequence conjugated to an albumin-binding domain are administered with an antigen. The antigen may be antigenic protein or polypeptide, or a fragment thereof (e.g., an epitope), or a nucleotide encoding the antigen or fragment thereof.

The antigen can be 2-100 amino acids (e.g., between 2 and 90, 2 and 80, 2 and 70, 2 and 60, 2 and 50, 2 and 40, 2 and 30, 2 and 20, 2 and 10, 10 and 100, 20 and 100, 30 and 100, 40 and 100, 50 and 100, 60 and 100, 70 and 100, 80 and 100, or 90 and 100 amino acids), including for example, 5 amino acids, 10 amino acids, 15 amino acids, 20 amino acids, 25 amino acids, 30 amino acids, 35 amino acids, 40 amino acids, 45 amino acids, or 50 amino acids. In some embodiments, a peptide can be greater than 50 amino acids. In some embodiments, the peptide can be >100 amino acids.

The protein or polypeptide can be any protein or peptide that can induce or increase the ability of the immune system to develop antibodies and T-cell responses to the protein or peptide.

Antigens can be peptides, proteins, polysaccharides, saccharides, lipids, nucleic acids, or combinations thereof. The antigen can be derived from a virus, bacterium, parasite, plant, protozoan, fungus, tissue or transformed cell such as a cancer or leukemic cell and can be a whole cell or immunogenic component thereof, e.g., cell wall components or molecular components thereof. Suitable antigens are known in the art and are available from commercial government and scientific sources. In one embodiment, the antigens are whole inactivated or attenuated organisms. These organisms may be infectious organisms, such as viruses, parasites and bacteria. These organisms may also be tumor cells. The antigens may be purified or partially purified polypeptides derived from tumors or viral or bacterial sources. The antigens can be recombinant polypeptides produced by expressing DNA encoding the polypeptide antigen in a heterologous expression system. The antigens can be DNA encoding all or part of an antigenic protein. The DNA may be in the form of vector DNA such as plasmid DNA.

Antigens may be provided as single antigens or may be provided in combination. Antigens may also be provided as complex mixtures of polypeptides or nucleic acids. Exemplary antigens are provided below.

### Viral Antigens

A viral antigen can be isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Cauliniovirus, Circoviridae, Closterovirus, Comoviridae, Coronaviridae (e.g., Coronavirus, such as severe acute respiratory syndrome (SARS) virus; e.g., SARS-CoV-2), Corticoviridae, Cystoviridae. Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4). Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g. measles, mumps, and human respiratory syncytial Virus), Parvoviridae, Picornaviridae (e.g. poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

Viral antigens may be derived from a particular strain such as a papilloma virus, a herpes virus, e.g., herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

### Bacterial Antigens

Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatiui, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptospirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonelia, Shigella, Spirillum, Spirochaeta, Staphylococcus, Streptococcus, Streptomyces, Sulfolobus, Thermoplasma, Thiobacillus, and Treponema, Vibrio, and Yersinia.

### Parasite Antigens

Parasite antigens can be obtained from parasites such as, but not limited to, an antigen derived from Cryptococcus neoformans, Histoplasma capsulatum, Candida albicans. Candida tropicalis, Nocardia asteroicdes, Rickettsia ricketsi, Rickettsia typhi, Mycoplasma pneumoniae, Chiamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

### Allergens and Environmental Antigens

The antigen can be an allergen or environmental antigen, such as, but not limited to, an antigen derived from naturally occurring allergens such as pollen allergens (tree-, herb, weed- and grass pollen allergens), insect allergens (inhalant, saliva and venom allergens), animal hair and dandruff allergens, and food allergens, Important pollen allergens from trees, grasses and herbs originate from the taxonomic orders of Fagales, Oleales, Pinales and piatanaceae including i.a. birch (Betula), alder (Alnus), hazel (Corylus), hornbeam (Carpinus) and olive (Olea), cedar (Cryptomeria and Juniperus), Plane tree (Platanus), the order of Poales including e.g., grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other allergen antigens that may be used include allergens from house dust mites of the genus Dermatophagoides and Euroglyphus, storage mite e.g. Lepidoglyphys, Glycyphagus and Tyrophagus, those from cockroaches, midges and fleas e.g. Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

### Cancer Antigens

A cancer antigen is an antigen that is typically expressed preferentially by cancer cells (i.e. it is expressed at higher levels in cancer cells than on non-cancer cells) and in some instances it is expressed solely by cancer cells. The cancer antigen may be expressed within a cancer cell or on the surface of the cancer cell. The cancer antigen may be a tumor-associated antigen. The cancer antigen can be MART-1/Melan-A, gp100, adenosine deaminase-binding protein (ADAbp), FAP, cyclophilin b, colorectal associated antigen (CRC)-0017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4). MAGE-C1, MAGE-C2, MAGE-C3, MAGE-C4, MAGE-05). GAGE-1, GAGE-2, GAGE-3, GAGE-4, GAGE-5, GAGE-6, GAGE-7, GAGE-8, GAGE-9, BAGE, RAGE, LAGE-1, NAG, GnT-V, MUM-1, CDK4, tyrosinase, p53, MUC family, HER2/neu, p21 ras, RCAS1, α-fetoprotein, E-cadherin, α-catenin, β-catenin, γ-catenin, p120ctn, gp100Pmel117, PRAME, NY-ESO-1, cdc27, adenomatous polyposis coli protein (APC), fodrin, Connexin 37, Ig-idiotype, p15, gp75, GM2 ganglioside, CD2 ganglioside, human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, or c-erbB-2.

### Adjuvants

In some embodiments, a pharmaceutical composition described herein may be administered with one or more adjuvants. An adjuvant refers to a substance that cause stimulation of the immune system. In this context, an adjuvant is used to enhance an immune response to one or more antigens. An adjuvant may be administered to a subject before, in combination with, or after administration of the antigens. In some embodiments, an additional adjuvant is administered to the subject in combination with the poly-dA and/or poly-dT conjugated to an albumin-binding domain, the poly-dG and/or poly-dC conjugated to an albumin-binding domain, or ISD or immunostimulatory HSV sequence conjugated to an albumin-binding domain and the antigen, or a nucleic acid sequence encoding the same, described herein. In some embodiments, an adjuvant may be conjugated to an albumin-binding domain, e.g., a lipid. The adjuvant may be without limitation lipids (e.g., monophosphoryl lipid A (MPLA)), alum (e.g., aluminum hydroxide, aluminum phosphate); Freund's adjuvant; saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS1312, water-based nanoparticles combined with a soluble immunostimulant, Seppic), and CDNs (cyclic di-nucleotides).

Adjuvants may be toll-like receptor (TLR) ligands. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants 5 acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

### Pharmaceutical Compositions

Described herein are pharmaceutical compositions of the disclosure including any one of the poly-dA and/or poly-dT nucleic acid sequences, poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain, or ISD or immunostimulatory HSV sequence conjugated to an albumin-binding domain described herein and an antigen, or nucleic acid sequence encoding an antigen. In addition to a therapeutic amount of the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain, or ISD or immunostimulatory HSV sequence conjugated to an albumin-binding domain described herein and an antigen, or nucleic acid sequence encoding an antigen), the pharmaceutical compositions may contain a pharmaceutically acceptable carrier or excipient, which can be formulated by methods known to those skilled in the art. Pharmaceutically acceptable salts of the components are also included, as described herein. In other embodiments, pharmaceutical compositions of the invention may contain nucleic acid molecules encoding one or more antigens described herein (e.g., in a vector, such as a viral vector). The nucleic acid molecule encoding the antigen thereof described herein may be cloned into an appropriate expression vector, which may be delivered via well-known methods in gene therapy. The antigen may be an influenza antigen, or fragment thereof. For example, the antigen may be an influenza nucleoprotein, or fragment thereof. Specifically, the influenza nucleoprotein may comprise a polypeptide sequence having at least 85% (e.g., at least 86%, 87%, 88%, 89%, 90%, 91%, 92%, 93%, 94%, 95%, 96%, 97%, 98%, 99%, or 100%) sequence identity to:

(SEQ ID NO: 22)
MASQGTKRSYEQMETDGERQNATEIRASVGKMIGGIGRFYIQMCTE
LKLSDYEGRLIQNSLTIERMVLSAFDERRNKYLEEHPSAGKDPKK
TGGPIYRRVNGKWMRELILYDKEEIRRIWRQANNGDDATAGLTHM
MIWHSNLNDATYQRTRALVRTGMDPRMCSLMQGSTLPRRSGAAGA
AVKGVGTMVMELVRMIKRGINDRNFWRGENGRKTRIAYERMCNIL
KGKFQTAAQKAMMDQVRESRNPGNAEFEDLTFLARSALILRGSVA
HKSCLPACVYGPAVASGYDFEREGYSLVGIDPFRLLQNSQVYSLI
RPNENPAHKSQLVWMACHSAAFEDLRVLSFIKGTKVLPRGKLSTR
GVQIASNENMETMESSTLELRSRYWAIRTRSGGNTNQQRASAGQI
SIQPTFSVQRNLPFDRTTIMAAFNGNTEGRTSDMRTEIIRMMESA
RPEDVSFQGRGVFELSDEKAASPIVPSFDMSNEGSYFFGDNAEEY
DN

The influenza nucleoprotein may comprise a polypeptide sequence having at least 95% (e.g., at least 95%, 96%, 97%, 98%, 99%, or 100%) sequence identity to SEQ ID NO: 22. In some embodiments, the influenza nucleoprotein has a polypeptide sequence of SEQ ID NO: 22, or fragment thereof. The antigen may be a coronavirus antigen, or fragment thereof. For example, the antigen may be a coronavirus spike protein, or fragment thereof.

Acceptable carriers and excipients in the pharmaceutical compositions of the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain, or ISD or immunostimulatory HSV sequence conjugated to an albumin-binding domain described herein and an antigen, or a nucleic acid sequence encoding an antigen, are nontoxic to recipients at the dosages and concentrations employed. In certain embodiments, the formulation material(s) are for subcutaneous (s.c.) and/or intravenous (i.v.) administration. In some embodiments, administration is by inhalation or intranasal administration. In some embodiments, the formulation material(s) intraperitoneal, topical, or oral administration. In some embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In some embodiments, suitable formulation materials include, but are not limited to, amino acids (such as glycine, glutamine, asparagine, arginine or lysine); antimicrobials; antioxidants (such as ascorbic acid, methionine, sodium sulfite or sodium hydrogen-sulfite); buffers (such as borate, bicarbonate, Tris-HCl, citrates, HEPES, TAE, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, sucrosemannose or dextran); proteins (such as human serum albumin, gelatin, dextran, and immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as hexamethonium chloride, octadecyldimethylbenzyl ammonium chloride, resorcinol, and benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In some embodiments, the optimal pharmaceutical composition will be determined by one skilled in the art depending upon, for example, the intended route of administration, delivery format and desired dosage. See, for example, Remington's Pharmaceutical Sciences, supra. In some embodiments, such compositions may influence the physical state, stability, rate of in vivo release and rate of in vivo clearance of the amphiphilic conjugate.

In some embodiments, the primary vehicle or carrier in a pharmaceutical composition, including the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain, or ISD or immunostimulatory HSV sequence conjugated to an albumin-binding domain described herein and an antigen, or a nucleic acid sequence encoding an antigen, can be either aqueous or non-aqueous in nature. For example, in some embodiments, a suitable vehicle or carrier can be water for injection, physiological saline solution, or artificial cerebrospinal fluid, possibly supplemented with other materials common in compositions for parenteral administration. In some embodiments, the saline includes isotonic phosphate-buffered saline. In certain embodiments, neutral buffered saline or saline mixed with serum albumin are further exemplary vehicles. In some embodiments, pharmaceutical compositions include Tris buffer of about pH 7.0-8.5, or acetate buffer of about pH 4.0-5.5, which can further include sorbitol or a suitable substitute therefore. In some embodiments, a composition including the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain described herein and an antigen, or a nucleic acid sequence encoding an antigen can be prepared for storage by mixing the selected composition having the desired degree of purity with optional formulation agents (Remington's Pharmaceutical Sciences, supra) in the form of a lyophilized cake or an aqueous solution. Further, in some embodiments, a composition the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain described herein and an antigen, or a nucleic acid sequence encoding an antigen, can be formulated as a lyophilizate using appropriate excipients such as sucrose.

In some embodiments, the pharmaceutical composition may be selected for parenteral delivery. The preparation of such pharmaceutically acceptable compositions is within the ability of one skilled in the art.

In some embodiments, the formulation components are present in concentrations that are acceptable to the site of administration. In some embodiments, buffers are used to maintain the composition at physiological pH or at a slightly lower pH, typically within a pH range of from about 5 to about 8.

In some embodiments, when parenteral administration is contemplated, a therapeutic composition can be in the form of a pyrogen-free, parenterally acceptable aqueous solution including the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain described herein and an antigen, or a nucleic acid sequence encoding an antigen, in a pharmaceutically acceptable vehicle. In some embodiments, a vehicle for parenteral injection is sterile distilled water in which a poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain described herein and an antigen, or a nucleic acid sequence encoding an antigen, is formulated as a sterile, isotonic solution, properly preserved. In some embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In some embodiments, hyaluronic acid can also be used, and can have the effect of promoting sustained duration in the circulation. In some embodiments, implantable drug delivery devices can be used to introduce the desired molecule.

The pharmaceutical composition may be administered in therapeutically effective amount such as to induce an immune response. The therapeutically effective amount of the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain described herein and an antigen, or a nucleic acid sequence encoding an antigen, included in the pharmaceutical preparations may be determined by one of skill in art, such that the dosage (e.g., a dose within the range of 0.01-100 mg/kg of body weight) induces an immune response in the subject.

Vectors may be used as in vivo nucleic acid delivery vehicle include, but are not limited to, retroviral vectors, adenoviral vectors, poxviral vectors (e.g., vaccinia viral vectors, such as Modified Vaccinia Ankara (MVA)), adeno-associated viral vectors, and alphaviral vectors. In some embodiments, a vector can include internal ribosome entry site (IRES) that allows the expression of multiple coronavirus antigens (e.g., a coronavirus spike protein, a peptide thereof, or a nucleic acid sequence encoding the same) described herein. Other vehicles and methods for nucleic acid delivery are described in U.S. Pat. Nos. 5,972,707, 5,697,901, and 6,261,554, each of which is incorporated by reference herein in its entirety. Other methods of producing pharmaceutical compositions are described in, e.g., U.S. Pat. Nos. 5,478,925, 8,603,778, 7,662,367, and 7,892,558, all of which are incorporated by reference herein in their entireties.

In some embodiments, a pharmaceutical composition described herein may be administered with one or more adjuvants.

### Routes, Dosage, and Timing of Administration

Pharmaceutical compositions of the disclosure that contain the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain described herein and an antigen, or a nucleic acid sequence encoding an antigen, described herein as the therapeutic agents may be formulated for parenteral administration, subcutaneous administration, intravenous administration, intramuscular administration, intranasal administration, or inhalation. Methods of administering therapeutic proteins are known in the art. See, for example, U.S. Pat. Nos. 6,174,529, 6,613,332, 8,518,869, 7,402,155, and 6,591,129, and US Patent Application Publication Nos. US20140051634, WO1993000077, and US20110184145, the disclosures of which are incorporated by reference in their entireties.

One or more of these methods may be used to administer a pharmaceutical composition of the invention that contains a poly-dA and/or a poly-dT-amphiphile, a poly-dG and/or a poly-dC amphiphile, a poly-dA and/or poly-dT sequence, a poly-dG and/or poly-dC sequence, or ISD or immunostimulatory sequence conjugated to an albumin-binding domain and an antigen described herein (e.g., a coronavirus antigen such as a coronavirus spike protein, a peptide thereof, or a nucleic acid sequence encoding the same, or an influenza virus antigen). For injectable formulations, various effective pharmaceutical carriers are known in the art. See, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986). The dosage of the pharmaceutical compositions of the invention depends on factors including the route of administration and the physical characteristics, e.g., age, weight, general health, of the subject. Typically, the amount of a poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain and an antigen, or a nucleic acid sequence encoding an antigen, described herein contained within a single dose may be an amount that effectively induces an immune response in the subject without inducing significant toxicity. A pharmaceutical composition of the invention may include a dosage of a poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain and an antigen, or a nucleic acid sequence encoding an antigen, described herein ranging from 0.001 to 500 mg (e.g., 0.01, 0.05, 0.1, 0.2, 0.3, 0.5, 0.7, 0.8, 1 mg, 2 mg, 3 mg, 4 mg, 5 mg, 10 mg, 15 mg, 20 mg, 30 mg, 50 mg, 100 mg, 250 mg, or 500 mg) and, in a more specific embodiment, about 0.1 to about 100 mg. The dosage may be adapted by the clinician in accordance with the different parameters of the subject.

Pharmaceutical compositions of the invention that contain the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain described herein and an antigen, or a nucleic acid sequence encoding an antigen, may be administered to a subject in need thereof, for example, one or more times (e.g., 1-10 times or more) daily, weekly, monthly, biannually, annually, or as medically necessary.

In some embodiments, an influenza nucleoprotein is administered to the subject. In some embodiments, an mRNA encoding a coronavirus antigen (e.g., a coronavirus spike protein, a peptide thereof, or a nucleic acid sequence encoding the same) is administered to the subject. In some embodiments, the antigen and the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain are administered concurrently or essentially at the same time to the subject. The poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain and the antigen may be co-formulated, or they may be administered as two separate formulations. In some embodiments, the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain described herein and the antigen, or a nucleic acid sequence encoding an antigen, are administered sequentially. For example, the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain may be administered first and the antigen, or a nucleic acid sequence encoding the same, may be administered second, or, in some embodiments, the antigen, or a nucleic acid sequence encoding the same, may be administered first and the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain is administered second. In some embodiments, the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain and the antigen, or a nucleic acid sequence encoding an antigen are administered with a second adjuvant.

### Methods of Inducing an Immune Response

The disclosure provides methods of inducing an immune response against an antigen in subject. The method includes administering any one of the compounds described herein and an antigen to the subject.

In some embodiments, the disclosure provides a method of inducing an immune response against an antigen in subject by administering any one of the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain described herein and an antigen to the subject and further administering an adjuvant to the subject. In some embodimentsthe poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain may be administered without one or more additional adjuvants.

In some embodiments, the method includes administering to the subject 1) a therapeutically effective amount of the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain described herein, and 2) an antigen or a nucleic acid sequence encoding the same. In some embodiments, the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain and the antigen are administered substantially simultaneously. In some embodiments, the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain and the antigen are administered separately. In some embodiments, the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain is administered first, followed by administering of the antigen. In some embodiments, the antigen is administered first, followed by administering of the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain.

In some embodiments, the antigen is an influenza antigen, or fragment thereof. In some embodiments, the antigen is an influenza nucleoprotein, or fragment thereof. In some embodiments, the Influenza nucleoprotein comprises a polypeptide sequence having at least 85% (e.g., at least 86%, 87%, 88%, 89%, 90%, 91%, 92%, 93%, 94%, 95%, 96%, 97%, 98%, 99%, or 100%) sequence identity to SEQ ID NO: 22. In some embodiments, the Influenza nucleoprotein comprises a polypeptide sequence having at least 95% (e.g., at least 96%, 97%, 98%, 99%, or 100%) sequence identity to SEQ ID NO: 22. In some embodiments, the Influenza nucleoprotein comprises a polypeptide sequence of SEQ ID NO: 22. In some embodiments, the antigen is a coronavirus antigen, or fragment thereof. For example, the antigen is a coronavirus spike protein, or fragment thereof, or a coronavirus nucleocapsid protein, or fragment thereof.

In some embodiments, one or more of the components administered is a pharmaceutically acceptable salt of the indicated component, as described herein.

In some embodiments, the immune response is protective against an infection. For example, the immune response may be protective again an Influenza infection or a SARS-CoV-2 infection.

In some embodiments, the immune response is protective against Covid-19 disease.

In some embodiments, the disclosure provides a method of inducing an immune response against an antigen in subject by administering any one of the compounds or pharmaceutically acceptable salts described herein subcutaneously to the subject. In some embodiments, the disclosure provides a method of inducing an immune response against an antigen in subject by administering the antigen intramuscularly, subcutaneously, intravenously, intraperitoneally, topically, or orally to the subject.

In some embodiments, the subject is a mammal. For the example, the subject may be a human.

### Kits

A kit can include the poly-dA and/or poly-dT nucleic acid sequences, the poly-dG and/or poly-dC nucleic acid sequences, the poly-dA and/or poly-dT nucleic acid sequences conjugated to an albumin-binding domain, or the poly-dG and/or poly-dC nucleic acid sequences conjugated to an albumin-binding domain, as disclosed herein, an antigen or nucleic acid encoding an antigen, and instructions for use. A kit may also include an ISD or immunostimulatory HSV sequence conjugated to an albumin-binding domain, as disclosed herein, an antigen or nucleic acid encoding an antigen, and instructions for use. The kits may include, in a suitable container, a poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain, an antigen or a nucleic acid encoding an antigen, one or more controls, and various buffers, reagents, enzymes and other standard ingredients well known in the art. In some embodiments, the kits further include an adjuvant. Accordingly, in some embodiments, the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain, and the antigen, or nucleic acid encoding an antigen, are in the same vial. In some embodiments, the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain, and the antigen, or nucleic acid encoding an antigen are in separate vials. Furthermore, in some embodiments, the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain, and the adjuvant are in the same vial. In some embodiments, the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain, and the adjuvant are in separate vials. In some embodiments, the antigen, or nucleic acid encoding the antigen, and adjuvant are in the same vial. In some embodiments, the antigen, or nucleic acid encoding the antigen, and the adjuvant are in separate vials.

The container can include at least one vial, well, test tube, flask, bottle, syringe, or other container means, into which the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain may be placed, and in some instances, suitably aliquoted. When an additional component is provided, the kit can contain additional containers into which this compound may be placed. The kits can also include a means for containing the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain, an antigen or nucleic acid encoding an antigen, and any other reagent containers in close confinement for commercial sale. Such containers may include injection or blow-molded plastic containers into which the desired vials are retained. Containers and/or kits can include labeling with instructions for use and/or warnings.

In some embodiments, the disclosure provides a kit including a medicament including a composition including a poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain, an antigen or nucleic acid encoding an antigen, an optional pharmaceutically acceptable carrier, and a package insert including instructions for administration of the medicament alone or in combination with a composition including an adjuvant and an optional pharmaceutically acceptable carrier, for treating, delaying progression of, or preventing a disease or condition (e.g., Influenza or SARS-CoV-2), wherein the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain, and optionally a linker. In some embodiments, the antigen may be an influenza antigen, or fragment thereof. In some embodiments, the antigen may be an influenza nucleoprotein, or fragment thereof. In some embodiments, the antigen may be a coronavirus antigen, or fragment thereof.

In some embodiments, the disclosure provides a kit including a container including a composition including a poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain, an antigen or nucleic acid encoding an antigen, an optional pharmaceutically acceptable carrier, and a package insert including instructions for administration of composition vaccine in a subject, wherein the poly-dA and/or poly-dT nucleic acid sequences, the poly-dG and/or poly-dC nucleic acid sequences, the poly-dA and/or poly-dT nucleic acid sequences conjugated to an albumin-binding domain, the poly-dG and/or poly-dC nucleic acid sequences conjugated to an albumin-binding domain or ISD or immunostimulatory HSV sequence conjugated to an albumin-binding domain optionally includes a linker. In some embodiments, the kit further includes an adjuvant and instructions for administration of the adjuvant.

In some embodiments of the kits, one of more of the components of the kits is a pharmaceutically acceptable salt of the component as described herein.

### EXAMPLES

The following examples, which are intended to illustrate, rather than limit, the disclosure, are put forth to provide those of ordinary skill in the art with a description of how the compositions and methods described herein may be used, made, and evaluated. The examples are intended to be purely exemplary of the disclosure and are not intended to limit the scope of what the inventors regard as their invention.

### Example 1. Evaluating the Activity of Single Versus Double-Stranded Amphiphilic DNA

The effects of single stranded DNA versus double stranded DNA dAdT constructs on the immune response were determined.

11 groups of 5 C57BL-6J mice each were administered a vaccine having the components described in Table 1.

TABLE 1
Summary of Vaccine Administration in Mice

| | | | | Dosing and Sample Collection | | | |
|---|---|---|---|---|---|---|---|
| | | Vaccine Components | | Day 0 | Day 13 | Day 14 | Day 21 |
| Group | Treatment Name | Antigen (5 ug) | Adjuvant (5 nmol) | Dose 1 | Read-out | Dose 2 | Read-out |
| 1 | dA/dT-50-PS | GenScript RBD | 5′-dT50-3′ | x | ICS | x | ICS on |
| | | | 5′-dA50-3′ | | | | Lung and |
| 2 | AMP-dA/dT-50-PS | GenScript RBD | 5′-AMP-dT50-3′ | x | | x | PBMCs |
| | | | 5′-dA50-3′ | | | | Spleen |
| 3 | AMP-dT/dA-50-PS | GenScript RBD | 5′-dT50-3′ | x | | x | ELISpot |
| | | | 5′-AMP-dA50-3′ | | | | |
| 4 | dA-50-PS | GenScript RBD | 5′-dA50-3′ | x | | x | |
| 5 | AMP-dA-50-PS | GenScript RBD | 5′-AMP-dA50-3′ | x | | x | |
| 6 | dT-50-PS | GenScript RBD | 5′-dT50-3′ | x | | x | |
| 7 | AMP-dT-50-PS | GenScript RBD | 5′-AMP-dT50-3′ | x | | x | |
| 8 | Mock (PBS) | GenScript RBD | — | x | | x | |

Adjuvant stock solutions were resuspended in limulus amebocyte lysate (LAL) H₂O, and final injections were diluted with 1×Phosphate-buffered saline (PBS), such that the amphiphilic and soluble dAdT had a concentration of 5 nmol/100 μL injection. The SARS-CoV2 Spike S1 RBD protein stock solutions were dissolved in PBS at a concentration of 0.88 mg/ml, and final injections were diluted with 1×PBS to a concentration of 5 μg/100 μL injection. The solutions were prepared with the vaccine components described in Table 2.

Immunizations were administered subcutaneously (SC) into the tail base of female B6 mice, bilaterally with 50 μL per side, and a booster dose was given at roughly 2-week intervals. SC injections ensured that the vaccine was optimally delivered into lymph nodes via natural lymph drainage, and bi-weekly injections were determined to be optimal in immune response.

TABLE 2
Vaccine Components

| Vaccine Components | Sequence or Cat# | Source | Lot # |
|---|---|---|---|
| SARS-COV2 RBD, His | Z03483 | GenScript | B200931 |
| dA/dT-50-PS | 5′-dT-3′ | AXO | XD-15740 K2 |
| | 5′-dA-3′ | | |
| AMP-dA/dT-50-PS | 5′-AMP-dT-3′ | AXO | XD-15739 K3 |
| | 5′-dA-3′ | | |
| AMP-dT/dA-50-PS | 5′-dT-3′ | AXO | XD-28896 K1 |
| | 5′-AMP-dA-3′ | | |
| dA-50-PS | 5′-dA-3′ | AXO | X-51174 K5-V2 |
| AMP-dA-50-PS | 5′-AMP-dA-3′ | AXO | X-86267 K1 |
| dT-50-PS | 5′-dT-3′ | AXO | X-51176 K1-V4 |
| AMP-dT-50-PS | 5′-AMP-dT-3′ | AXO | X-51175 K1 |

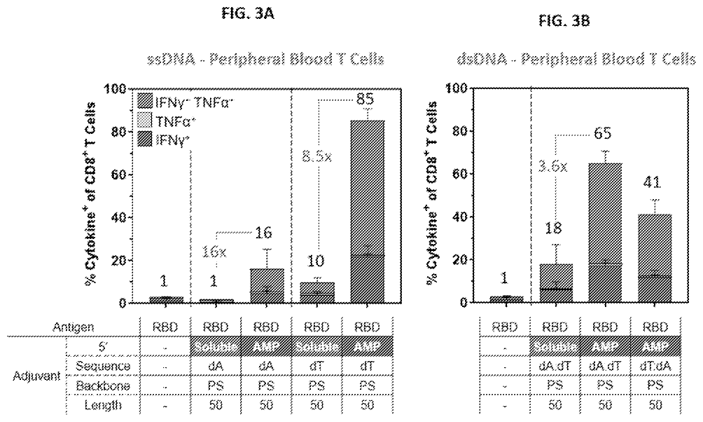

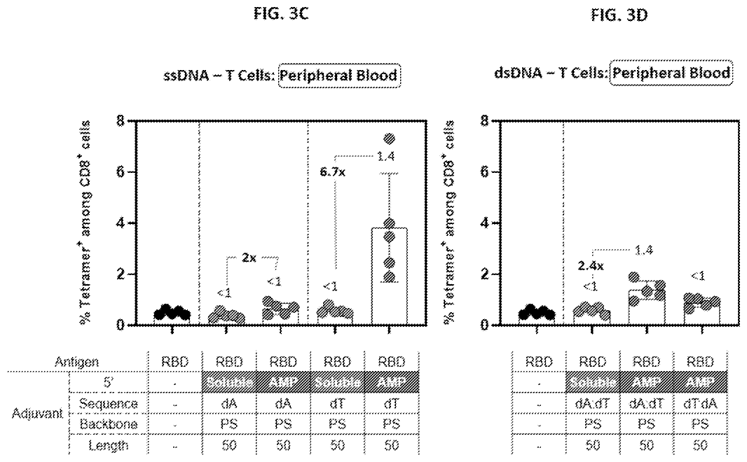

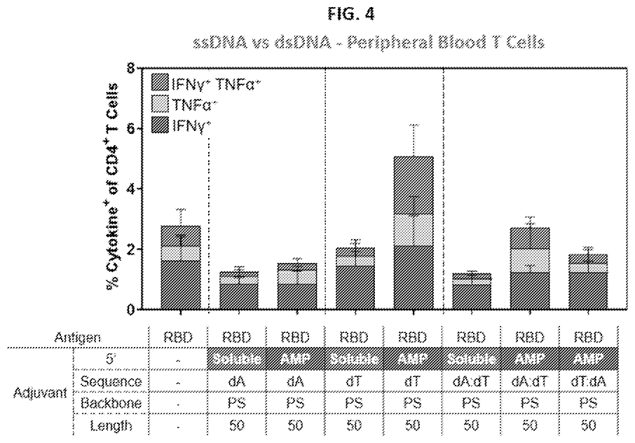

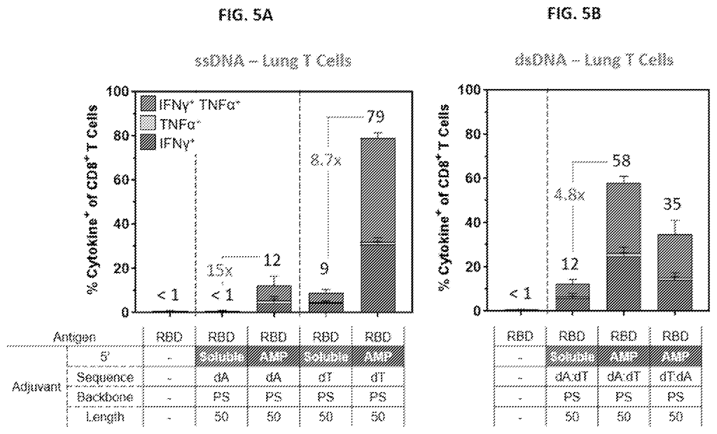

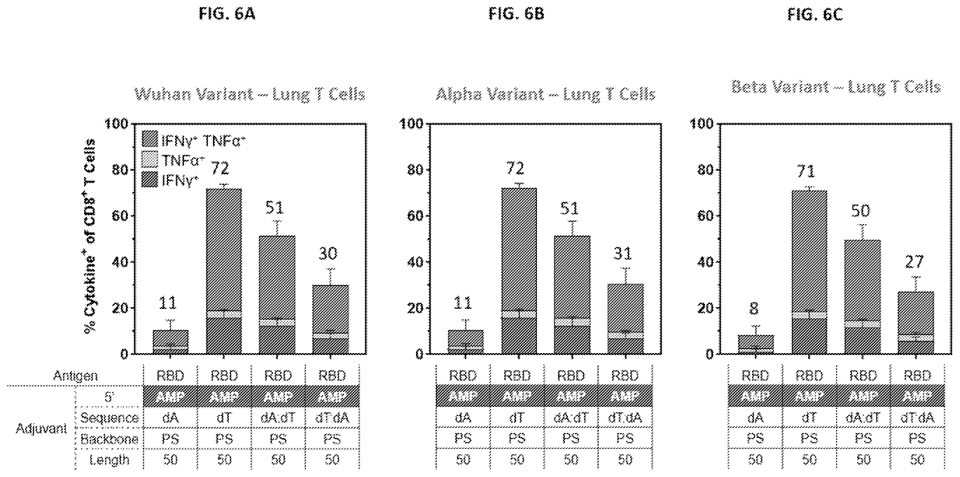

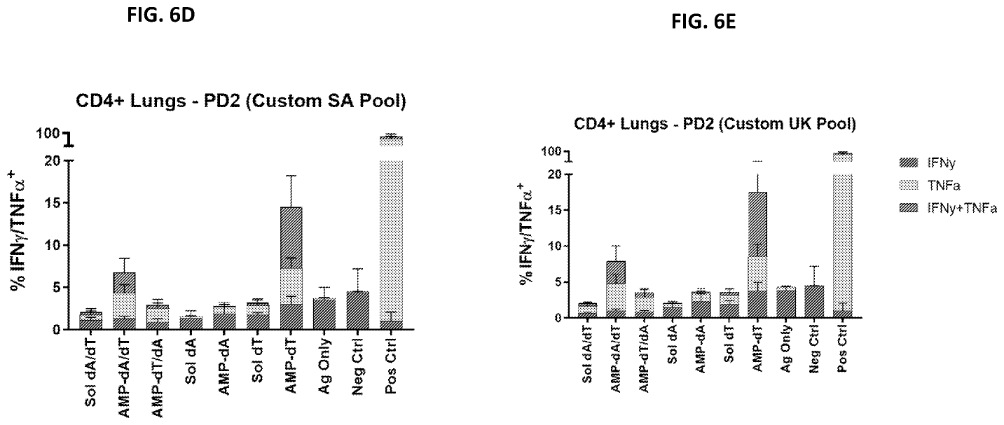

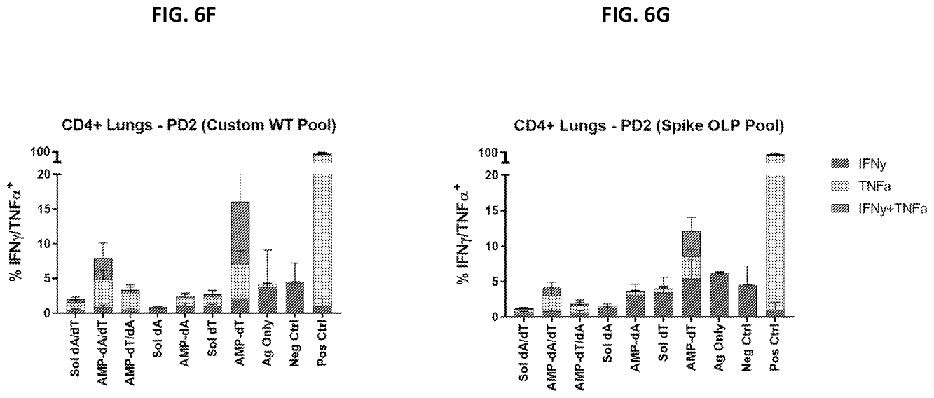

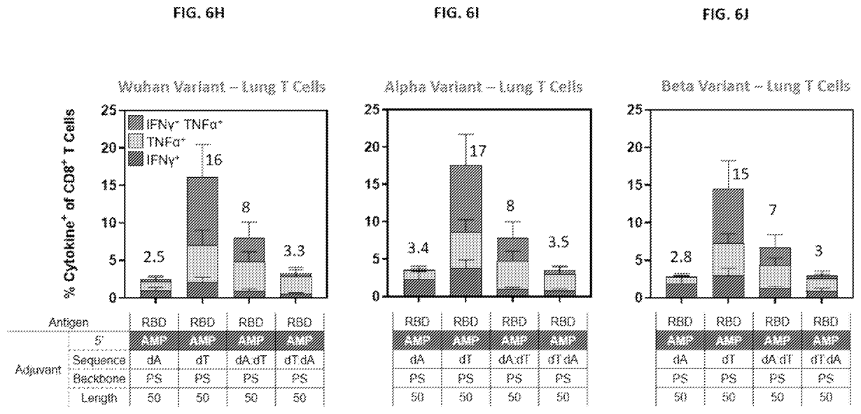

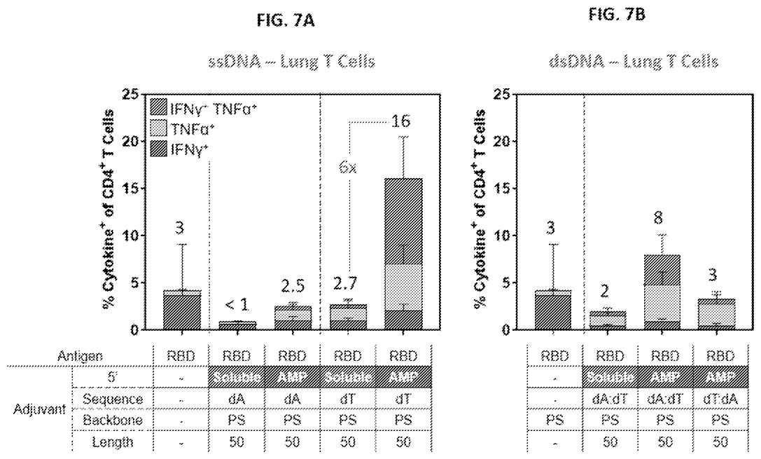

An ICS (Intracellular Stain) Assay for TNFα and IFNγ was performed on PBMCs 7 days after dosing 2 (FIGS. 3A-31B and 4). ICS was also performed on lung samples 7 days post dose 2 (FIGS. 5A, 51B, 6A-6J, 7A, and 7B). The cells were surface stained for CD4, CD8, and CD3. The antibodies described in Table 3 were used for the ICS assay. The ICS samples were activated overnight (in the presence of Brefeldin A and Monensin) with 1 μg/well of SARS-CoV-2 Spike Glycoprotein Peptide Pool Mix [315 peptides each at 1 μg/well](Table 4).

TABLE 3
Antibodies used for ICS

| Antigen | Color | Source | Product # | Lot # |
|---|---|---|---|---|
| TNFα | FITC | BD | 554418 | 9123915 |
| IFNγ | PE | BD | 554412 | 9154769 |
| CD8a | APC | eBioscience | 17-0081-83 | 4321418 |
| CD4 | PE-Cy7 | Invitrogen | 25-0041-82 | 2123767 |
| CD3 | APC-Cy7 | BD | 560590 | 9179637 |
| LiveDead | Aqua | Invitrogen | L34966 | 1832692 |
| Brefeldin A | — | Invitrogen | 00-4506-51 | 1915300 |
| Monensin | — | BioLegend | 420701 | B297750 |

TABLE 4
Re-Stimulation Peptides

| Re-stimulation | | | |
|---|---|---|---|
| Peptides | Sequence | Source | Lot # |
| SARS-COV-2 Spike | 315 15mers | GenScript | E5868620K |
| Glycoprotein | spanning Spike | | |
| Peptide Pool Mix | Protein Sequence, | | |
| | overlap 11aa | | |

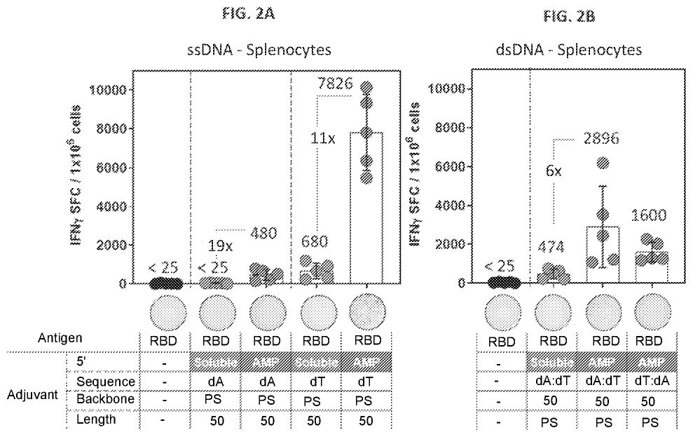

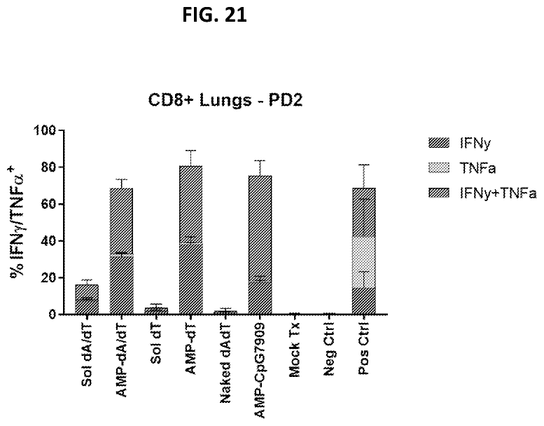

ELISpot analysis for IFNγ was performed on splenocytes after dose 2 administration. Splenocytes (0.1×10⁶ cells/well) were activated with 1 μg/well SARS-CoV-2 Spike Glycoprotein Peptide Pool Mix (Table 4), and the IFNy plates were stimulated overnight (FIGS. 2A and 21B). In another experiment, cells were restimulated with 4 different peptide pools: 1) those described in Table 4 (restimulation), 2) a custom-made peptide pool made by GenScript that is identical to the one above, 3) a custom-made peptide pool made by GenScript that has corresponding mutations for the UK variant B.1.1.7, and 4) a custom-made peptide pool made by GenScript that has corresponding mutations for the South Africa variant B.1.351.

The new restimulation peptide pools for the SARS-CoV2 Spike RBD that were custom made for the purpose of being able to substitute single 15Smer peptides to match different CoV2 strains were assessed. The custom pool for the WT strain yielded the same results as the commercially available pool from GenScript. Additionally, there were no differences in T-cell responses among the different peptide pools. This indicated that the T-cell responses were not affected by the various mutations between the tested strains.

Tetramer analysis was performed 7 days post dosing using the H-2Kb CoV2 RBD Tetramer-VNFNFNGL-PE (NIH 53309; SEQ ID NO:25) (FIGS. 3C and 3D).

Compared to the other adjuvants AMP-dT appeared to be the most immunogenic. The CD8 responses, as measured by tetramer, were already detectable after dose 1 of the vaccine. The ICS data post dose 2 from peripheral blood also indicated that AMP-dT elicited a robust T-cell response where 85% of CD8 cells and 5% of CD4 cells are positive for TNFa and IFNy cytokines. This surpassed even the immune response of AMP-dAdT, which resulted in 65% of CD8 cells and 1.2% of CD4 cells being positive for TNFa and IFNy cytokines. The same trend was seen in the ICS data from lung lymphocytes and ELISpot data from splenocytes. Serum antibody ELISA data showed that, AMP-dT, followed by AMP-dAdT, generated the greatest antibody response.

### Example 2. Evaluating Lymph Node Accumulation of Double-Stranded Amphiphilic DNA

The biodistribution of AMP-dAdT, its efficiency of getting into the lymph node, and its retention in the lymph node was determined.

4 groups of 10 C57BL-6J mice each were administered a vaccine including 5 μg of the SARS-CoV-2 RBD antigen and 5 nmol of AMP-dT and FAM-dA each 50 nucleotides in length, 5 μg of the SARS-CoV-2 RBD antigen and 5 nmol of dT and FAM-dA each 50 nucleotides in length, 5 μg of the SARS-CoV-2 RBD antigen and 5 nmol of AMP-dT and dA each 50 nucleotides in length, or PBS as described in Table 5. The lymph node and spleen of 5 mice were imaged after 1, 2 days, each.

TABLE 5
Summary of Vaccine Administration in Mice

| | | | | Dosing and Sample Collection | | |
|---|---|---|---|---|---|---|
| | | Vaccine Components | | Day 0 | Day 1 | Day 2 |
| Group | Treatment Name | Antigen (5 ug) | Adjuvant (5 nmol) | Dose 1 | Read-out | Read-out |
| 1 | AMP-dAdT-FITC | GenScript RBD | 5′-AMP-dT50-3′ | x | on 5 | on 5 |
| | | | 5′-FAM-dA50-3′ | | mice: IVIS | mice: IVIS |
| 2 | Sol dAdT-FITC | GenScript RBD | 5′-dT50-3′ | x | (Gr 1-3) | (Gr 1-3) |
| | | | 5′-FAM-dA50-3′ | | Flow | Flow |
| 3 | No FITC Ctrl | GenScript RBD | 5′-AMP-dT50-3′ | x | (all Gr) | (all Gr) |
| | | | 5′-dA50-3′ | | | |
| 4 | Neg Ctrl | GenScript RBD | PBS | x | | |

Adjuvant stock solutions were resuspended in limulus amebocyte lysate (LAL) H₂O, and the final injections were diluted with 1×Phosphate-buffered saline (PBS), such that the AMP-dT and FAM-dA, soluble dT and FAM-dA, and AMP-dT and soluble dA all had concentrations of 5 nmol/100 μL injection. The SARS-CoV2 Spike S1 RBD protein stock solutions were dissolved in PBS at a concentration of 0.88 mg/ml, and the final injections were diluted with 1×PBS to a concentration of 5 μg/100 μL injection. The solutions were prepared with the vaccine components described in Table 6.

The immunizations were administered subcutaneously (SC) into the tail base of female B6 mice, bilaterally with 50 μL per side. SC injections ensured that the vaccine was optimally delivered into lymph nodes via natural lymph drainage.

TABLE 6
Vaccine Components

| Vaccine | | | |
|---|---|---|---|
| Components | Sequence or Cat# | Source | Lot # |
| SARS-COV2 | Z03483 | GenScript | |
| RBD, His | | | |
| AMP-dAdT-FAM | 5′-(Diacyl lipid)-sdT(x50)-3′ | AXO | XD-17763 |
| | 5′-(FAM)sdA(x50)-3′ | | |
| dAdT-FAM | 5′-sdT(x50)-3′ | AXO | XD-17764 |
| | 5′-(FAM)sdA(x50)-3′ | | |
| AMP-dAdT | 5′-(Diacyl lipid)-sdT(x50)-3′ | AXO | XD-15739 K2 |
| | 5′-sdA(x50)-3′ | | |

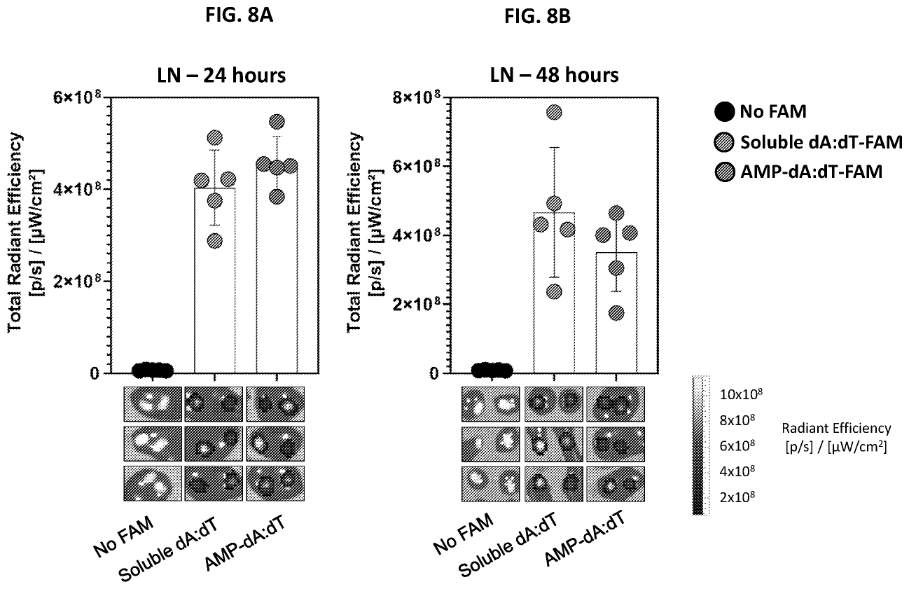

IVIS imaging was performed 24 hours and 48 hours post injection. The inguinal and auxiliary lymph nodes were harvested and imaged from 5 animals per timepoint. While imaging, the lymph nodes were kept wet with PBS. Total radiant efficiency in the lymph node was measured (FIGS. 8A and 8B).

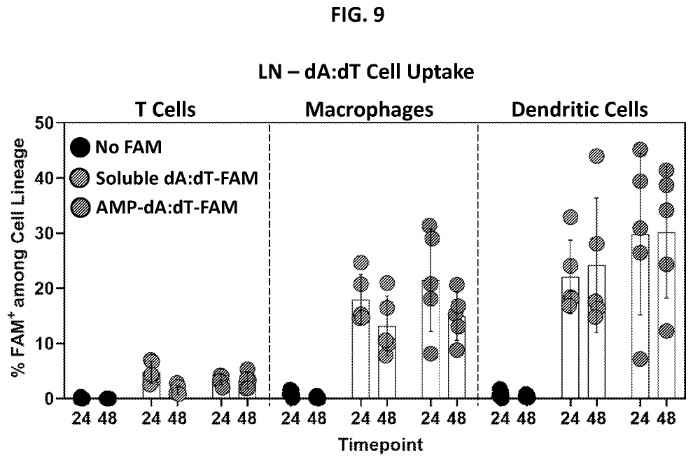

Flowcytometric analysis was performed on lymph nodes after IVIS imaging. The lymph nodes were processed and stained for cell surface markers distinguishing between DCs, Mϕ and B-cells using the antibodies described in Table 7, including T-cells: CD3⁺CD19⁻ CD11 b⁻, B-cells: CD3⁻ CD19⁺CD11c⁻ CD11b^{low &+}, and Pan mDCs: CD3⁻ CD19⁻ CD11c⁺CD11 b⁺MHCII⁺. The percent FAM⁺ found among cell lineage for T cells, macrophages, and dendritic cells at the 24 hour and 48 hour timepoint are summarized in FIG. 9.

TABLE 7
Antibody Panels

| FITC | Amph-constructs |
|---|---|
| PE | CD11b |
| PerCP-Cy5.5 | CD19 |
| APC | CD3 |
| PE-Cy7 | MHCII |
| APC-Cy7 | LiveDead |
| BV510 (AmCyan) | CD11c |

This experiment shows that amphiphilic and soluble adjuvants of such size (ca. 32 kDa) are being delivered to and retained in the lymph nodes to similar extent. Despite this, historically we have shown that the amphiphilic adjuvants achieved a much greater immune response than the soluble adjuvants, indicating additional pathways in which AMP-modifications facilitate the generation of an immune response.

### Example 3. Evaluating the Contribution of Length on Activity of Double-Stranded Amphiphilic DNA

The effects of the construct length of the dAdT complexes on the immune response was determined.

11 groups of 5 C57BL-6J mice each were administered a vaccine including the components described in Table 8.

TABLE 8
Summary of Vaccine Administration in Mice

| | | | | Dosing and Sample Collection | | | |
|---|---|---|---|---|---|---|---|
| | | Vaccine Components | | Day 0 | Day 13 | Day 14 | Day 21 |
| Group | Treatment Name | Antigen (5 ug) | Adjuvant (5 nmol) | Dose 1 | Read-out | Dose 2 | Read-out |
| 1 | Sol dAdT 30 | GenScript RBD | 5′-dAdT 30-3′ | x | ICS | x | ICS on |
| 2 | Sol dAdT 40 | GenScript RBD | 5′-dAdT 40-3′ | x | | x | Lung and |
| 3 | Sol dAdT 50 | GenScript RBD | 5′-dAdT 50-3′ | x | | x | PBMCs |
| 4 | Sol dAdT 75 | GenScript RBD | 5′-dAdT 75-3′ | x | | x | Spleen |
| 5 | Sol dAdT 100 | GenScript RBD | 5′-dAdT 100-3′ | x | | x | ELISpot |
| 6 | AMP dAdT 30 | GenScript RBD | 5′-AMP-dAdT 30-3′ | x | | x | |
| 7 | AMP dAdT 40 | GenScript RBD | 5′-AMP-dAdT 40-3′ | x | | x | |
| 8 | AMP dAdT 50 | GenScript RBD | 5′-AMP-dAdT 50-3′ | x | | x | |
| 9 | AMP dAdT 75 | GenScript RBD | 5′-AMP-dAdT 75-3′ | x | | x | |
| 10 | AMP dAdT 100 | GenScript RBD | 5′-AMP-dAdT 100-3′ | x | | x | |
| 11 | AMP dAdT 100 | — | 5′-AMP-dAdT 50-3′ | x | | x | |

Adjuvant stock solutions were resuspended in limulus amebocyte lysate (LAL) H₂O, and the final injections were diluted with 1×Phosphate-buffered saline (PBS) such that the AMP-dAdT and soluble dAdT of various lengths had a concentration of 5 nmol/100 μL injection. The SARS-CoV2 Spike S1 RBD protein stock solutions were dissolved in PBS at a concentration of 0.88 mg/ml. Final injections were diluted with 1×PBS to a concentration of 5 μg/100 μL injection. The vaccine components are described in Table 9.

The immunizations were administered subcutaneously (SC) into the tail base of female B6 mice, bilaterally with 50 μL per side. A booster dose was given at roughly 2-week intervals. SC injections ensured that the vaccine was optimally delivered into lymph nodes via natural lymph drainage, and bi-weekly injections were determined to be optimal in immune response.

TABLE 9
Vaccine Components

| Vaccine Components | Sequence or Cat# | Source | Lot # |
|---|---|---|---|
| SARS-CoV2 RBD, His | Z03483 | GenScript | |
| AMP-dAdT 30 | 5′-AMP-dT30-33′ | AXO | XD-29661 |
| | 5′-dA30-33′ | | |
| AMP-dAdT 40 | 5′-AMP-dT40-33′ | AXO | XD-29662 |
| | 5′-dA40-33′ | | |
| AMP-dAdT 50 | 5′-AMP-dT50-3′ (SEQ ID NO: 33) | AXO | XD-15739 |
| | 5′-dA50-3′ (SEQ ID NO: 30) | | |
| AMP-dAdT 75 | 5′-AMP-dT75-33′ | AXO | XD-29663 |
| | 5′-dA75-33′ | | |
| AMP-dAdT 100 | 5′-AMP-dT100-33′ | AXO | XD-29664 |
| | 5′-dA100-33′ | | |
| Sol dAdT 30 | 5′-dT30-33′ | AXO | XD-29665 |
| | 5′-dA30-33′ | | |
| Sol dAdT 40 | 5′-dT40-33′ | AXO | XD-29666 |
| | 5′-dA40-33′ | | |
| Sol dAdT 50 | 5′-dT50-3′ (SEQ ID NO: 32) | AXO | XD-15740 |
| | 5′-dA50-3′ (SEQ ID NO: 30) | | |
| Sol dAdT 75 | 5′-dT75-33′ | AXO | XD-29667 |
| | 5′-dA75-33′ | | |
| Sol dAdT 100 | 5′-dT100-33′ | AXO | XD-29668 |
| | 5′-dA100-3′ | | |

An ICS (Intracellular Stain) assay for TNFα and IFNγ was performed on PBMCs 7 days after dosing. An ICS was also performed on lung samples 7 days post dose 2 (FIGS. 11A, 11B, 12A, and 12B). The cells were also surface stained for CD4, CD8 and CD3 using the antibodies described in Table 10. ICS samples were activated overnight (in the presence of Brefeldin A and Monensin) with 1 μg/well of SARS-CoV-2 Spike Glycoprotein Peptide Pool Mix [315 peptides each at 1 μg/well](Table 11)

TABLE 10
Antibodies used for ICS

| Antigen | Color | Source | Product # | Lot # |
|---|---|---|---|---|
| TNFα | FITC | BD | 554418 | 9123915 |
| IFNγ | PE | BD | 554412 | 9154769 |
| CD8a | APC | eBioscience | 17-0081-83 | 4321418 |
| CD4 | PE-Cy7 | Invitrogen | 25-0041-82 | 2123767 |
| CD3 | APC-Cy7 | BD | 560590 | 9179637 |
| LiveDead | Aqua | Invitrogen | L34966 | 1832692 |
| Brefeldin A | — | Invitrogen | 00-4506-51 | 1915300 |
| Monensin | — | BioLegend | 420701 | B297750 |

TABLE 11
Re-Stimulation Peptides

| Re-stimulation | | | |
|---|---|---|---|
| Peptides | Sequence | Source | Lot # |
| SARS-CoV-2 Spike | 315 15mers spanning | GenScript | E5868620K |
| Glycoprotein | Spike Protein | | |
| Peptide Pool Mix | Sequence, overlap | | |
| | 11aa | | |

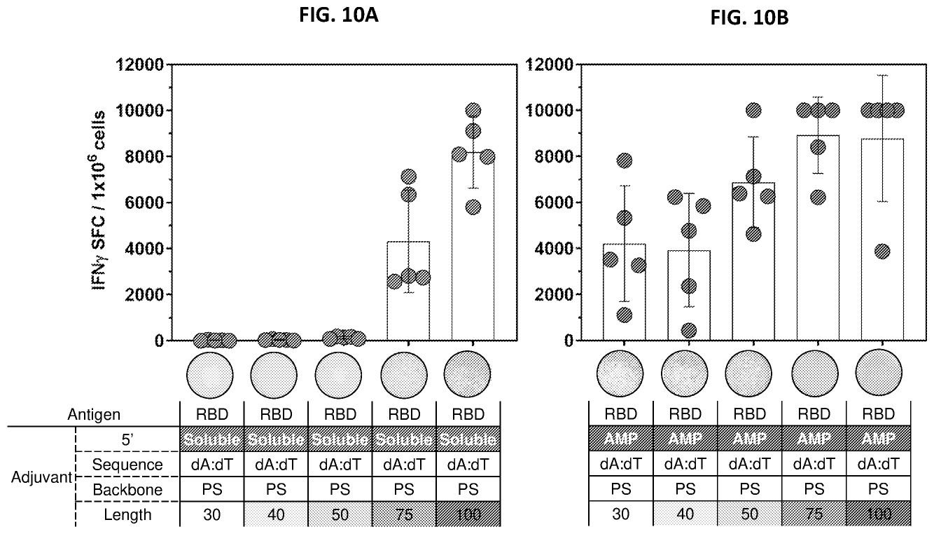

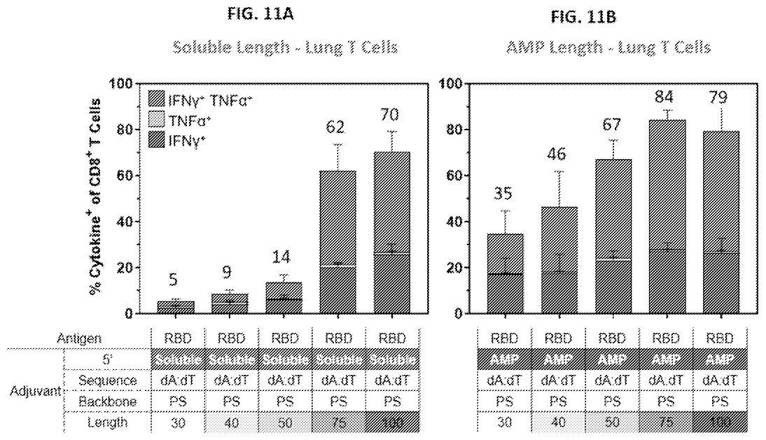

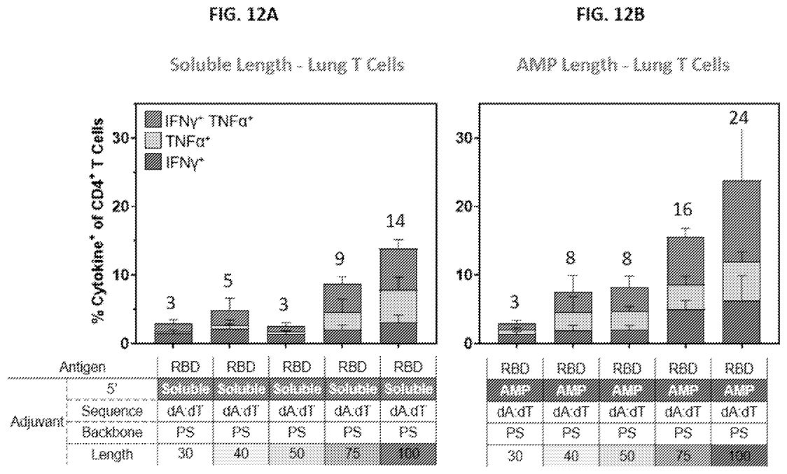

ELISpot analysis for IFNγ was performed on splenocytes after dose 2 administration (FIGS. 10A and 10B). The splenocytes (0.25×10⁶ cells/well) were activated with 1 μg/well Peptide Pool Mix (Table 11). The IFNγ plates were stimulated overnight.

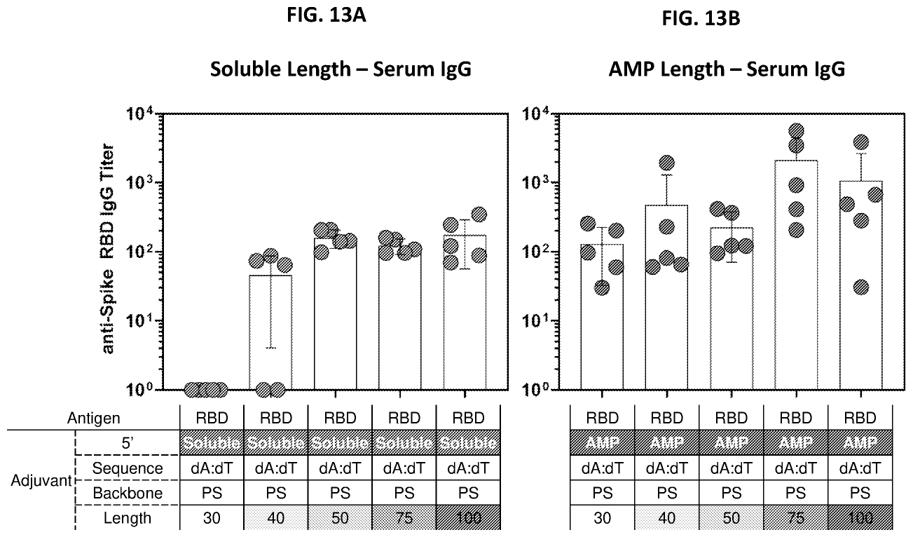

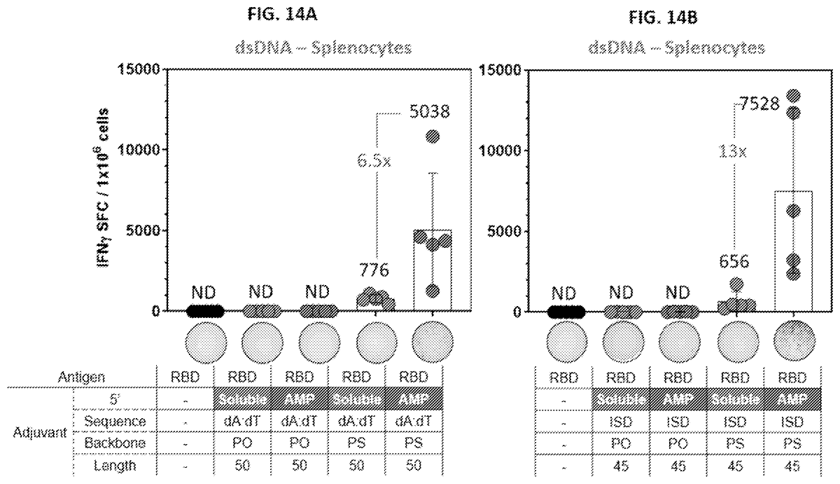

SARS-CoV2 specific serum ELISA (enzyme-linked immunosorbent assay) was performed on mouse serum 7 days after each dose, to detect any RBD-specific antibody response (FIGS. 13A and 13B). Whole blood was collected and then centrifuged using Ser-gel tubes (NC9436363, Fisher Scientific). The serum was either used fresh or stored at −80° C. until used. 96-well plates were coated with 200 ng/100 μL (2 μg/ml) of CoV2 RBD protein (Z03483, GenScript) and left overnight at 4° C. The plates were then pre-blocked with 2% BSA for 2 hours at room temperature. The mouse serum was diluted to 1:20 and then serially diluted (1:5→8 concentrations) in a dummy plate. The ELISA plates were washed once with ELISA washing buffer (BioLegend 4211601). The samples were transferred to the ELISA plate and incubated for 2 hours at room temperature. The plates were washed 4 times with washing buffer. For serum antibody detection, the secondary HRP-conjugated antibodies in Table 12 were used at 1:2000 in PBS+ and incubated for 1 hour at room temperature, and the plates were washed 4 times with washing buffer. The reaction was visualized by addition of substrate 3,3′,5,5′-Tetramethylbenzidine (TMB) for 10 minutes at room temperature and stopped by H2SO4 (1 N). The absorbance at 450 nm was measured by an ELISA plate reader.

TABLE 12
Secondary HRP-conjugated antibodies

| Antigen | Source | Product # | Lot # |
|---|---|---|---|
| IgG | Jackson Immunoresearch | 315-035-046 | 147406 |

For the amphiphilic adjuvants, the T-cells showed the greatest response when the adjuvant was either 100 or 75 nucleotides in length, followed by those having 50 nucleotides, followed by those having 40 nucleotides, and then followed by those having 30 nucleotides. For the soluble adjuvants, the T-cells showed the greatest response when the adjuvant was with 100 or 75 nucleotides, followed by those having 50, 40, or 30 nucleotides in length. At lower lengths, including 30, 40, and 50 nucleotides, the amphiphilic adjuvants generated the greater T-cell response compared to their soluble counterparts. At higher lengths, including 75 and 100 nucleotides, the amphiphilic adjuvants and the soluble adjuvants generated a similar T-cell response.

Amphiphilic adjuvants of all lengths resulted in a similar antibody response. Soluble adjuvants having a length of 40, 50, 75, or 100 nucleotides resulted in a similar antibody response, while the soluble adjuvant of 30 nucleotides was inactive. Overall, the amphiphilic adjuvants resulted in a greater antibody response than the soluble counterparts.

Based on the ICS and ELISpot results, the AMP-modification of the dAdT construct was beneficial to eliciting an immune response. Especially, when looking at responses in the lung, the AMP-tail enabled smaller constructs to robustly elicit immune responses. Additionally, AMP increases the response elicited by dAdT in comparison to soluble dAdT.

### Example 4. Evaluating the Activity of Phosphorothioate Versus Phosphodiester Double Stranded DNA

The effects of phosphodiester (P0) versus phosphorothioate (PS) in the double-stranded DNA constructs on the immune response was determined.

9 groups of 5 C57BL-6J mice each were administered a vaccine including the components described in Table 13.

TABLE 13
Summary of Vaccine Administration in Mice

| | | | | Dosing and Sample Collection | | | |
|---|---|---|---|---|---|---|---|
| | | Vaccine Components | | Day 0 | Day 13 | Day 14 | Day 21 |
| Group | Treatment Name | Antigen (5 ug) | Adjuvant (5 nmol) | Dose 1 | Read-out | Dose 2 | Read-out |
| 1 | dA/dT-50-PS | GenScript RBD | 5′-sdT50-3′ (PS) | x | ICS | x | ICS on |
| | | | 5′-sdA50-3′ (PS) | | | | Lung and |
| 2 | AMP-dA/dT-50-PS | GenScript RBD | 5′-AMP-dT50-3′ (PS) | x | | x | PBMCs |
| | | | 5′-dA50-3′ (PS) | | | | Spleen |
| 3 | dA/dT-50-PO | GenScript RBD | 5′-sdT50-3′ (PO) | x | | x | ELISpot |
| | | | 5′-sdA50-3′ (PO) | | | | |
| 4 | AMP-dA/dT-50-PO | GenScript RBD | 5′-AMP-dT50-3′ (PO) | x | | x | |
| | | | 5′-dA50-3′ (PO) | | | | |
| 5 | ISD-PS | GenScript RBD | 5′-ISD-3′ (PS) | x | | x | |
| | | | comp strand (PS) | | | | |
| 6 | AMP-ISD-PS | GenScript RBD | 5′-AMP-ISD-3′ (PS) | x | | x | |
| | | | comp strand (PS) | | | | |
| 7 | ISD-PO | GenScript RBD | 5′-ISD-3′ (PO) | x | | x | |
| | | | comp strand (PO) | | | | |
| 8 | AMP-ISD-PO | GenScript RBD | 5′-AMP-ISD-3′ (PO) | x | | x | |
| | | | comp strand (PO) | | | | |
| 9 | Mock Tx | GenScript RBD | — | x | | x | |

Adjuvant stock solutions were resuspended in limulus amebocyte lysate (LAL) H₂O, and the final injections were diluted with 1×Phosphate-buffered saline (PBS) such that the AMP-dAdT and soluble dAdT of PO and PS variation had a concentration of 5 nmol/100 μL injection. The SARS-CoV2 Spike 51 RBD protein stock solutions were dissolved in PBS at a concentration of 0.88 mg/ml. Final injections were diluted with 1×PBS to a concentration of 5 μg/100 μL injection. The vaccine components are described in Table 14.

The immunizations were administered subcutaneously (SC) into the tail base of female B6 mice, bilaterally with 50 μL per side. A booster dose was given at roughly 2-week intervals. SC injections ensured that the vaccine was optimally delivered into lymph nodes via natural lymph drainage, and bi-weekly injections were determined to be optimal in immune response.

TABLE 14
Vaccine Components

| Vaccine Components | Sequence or Cat# | Source | Lot # |
|---|---|---|---|
| SARS-CoV2 RBD, His | Z03483 | GenScript | B200931 |
| dA/dT-50-PS | 5′-sdT-33′ | AXO | XD-15740 K2 |
| | 5′-sdA-33′ | | |
| AMP-dA/dT-50-PS | 5′-AMP-sdT-33′ | AXO | XD-15739 K3 |
| | 5′-sdA-33′ | | |
| dA/dT-50-PO | 5′-dT-33′ | AXO | XD-28696 |
| | 5′-dA-33′ | | |
| AMP-dA/dT-50-PO | 5′-AMP-dT-33′ | AXO | XD-28697 |
| | 5′-dA-33′ | | |
| ISD-PS | ISD PS | AXO | XD-29671 |
| | Comp strand PS | | |
| AMP-ISD-PS | AMP-ISD PS | AXO | XD-29672 |
| | Comp strand PS | | |
| ISD-PO | ISD PO | AXO | XD-18243 |
| | Comp strand PO | | |
| AMP-ISD-PO | AMP-ISD PO | AXO | XD-18244 |
| | Comp strand PO | | |

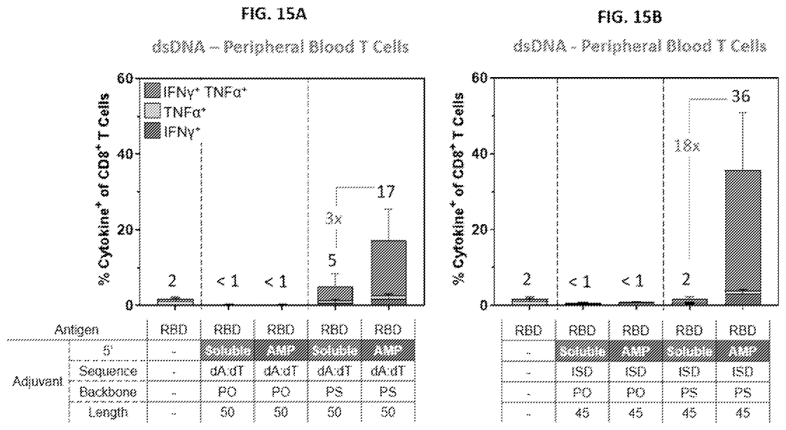

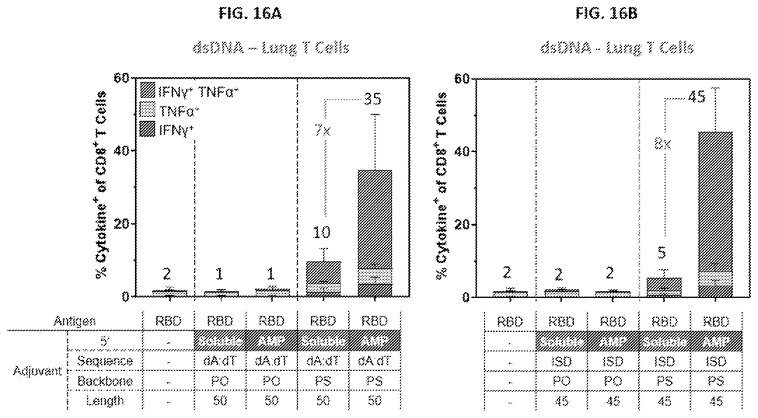

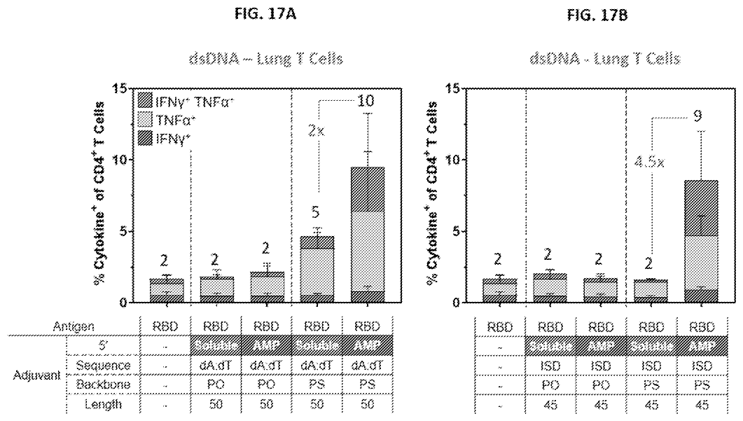

An ICS (Intracellular Stain) Assay for TNFα and IFNγ was performed on PBMCs 7 days after 5 dosing (FIGS. 15A and 15B). An ICS was also performed on lung samples 7 days post dose 2 (FIGS. 16A, 16B, 17A, and 17B). The cells were surface stained for CD4, CD8 and CD3 using the antibodies from Table 15. The ICS samples were activated overnight (in the presence of Brefeldin A and Monensin) with 1 μg/well of SARS-CoV-2 Spike Glycoprotein Peptide Pool Mix [315 peptides each at 1 μg/well](Table 16).

TABLE 15
Antibodies used for ICS

| Antigen | Color | Source | Product # | Lot # |
|---|---|---|---|---|
| TNFα | FITC | BD | 554418 | 9123915 |
| IFNγ | PE | BD | 554412 | 9154769 |
| CD8a | APC | eBioscience | 17-0081-83 | 4321418 |
| CD4 | PE-Cy7 | Invitrogen | 25-0041-82 | 2123767 |
| CD3 | APC-Cy7 | BD | 560590 | 9179637 |
| LiveDead | Aqua | Invitrogen | L34966 | 1832692 |
| Brefeldin A | — | Invitrogen | 00-4506-51 | 1915300 |
| Monensin | — | BioLegend | 420701 | B297750 |

TABLE 16
Re-Stimulation Peptides

| Re-stimulation | | | |
|---|---|---|---|
| Peptides | Sequence | Source | Lot # |
| SARS-CoV-2 Spike | 315 15mers | GenScript | E5868620K |
| Glycoprotein | spanning Spike | | |
| Peptide Pool Mix | Protein Sequence, | | |
| | overlap 11aa | | |

ELISpot analysis for IFNγ was performed on splenocytes after dose 2 administration (FIGS. 14A and 14B). Splenocytes (0.1×10⁶ cells/well) were activated with 1 μg/well Peptide Pool Mix (Table 16). The IFNγ plates were stimulated overnight. For serum antibody detection, the secondary HRP-conjugated antibodies in Table 17 were used.

TABLE 17
Secondary HRP-conjugated antibodies

| Antigen | Source | Product # | Lot # |
|---|---|---|---|
| IgG | Jackson Immunoresearch | 315-035-046 | 147406 |
| IgG1 | Jackson Immunoresearch | 115-035-205 | 148255 |
| IgG2c | Jackson Immunoresearch | 115-035-2081 | 146880 |

When generating the DNA-based adjuvants, it was important to use a phosphorothioate (PS) backbone, as the phosphodiester (PO), which is the natural occurring bond between nucleic acids, had much lower adjuvanticity. This could be because naturally occurring DNA can be degraded more easily by endogenous endonucleases. The highest T-cell responses were measured for AMP-dAdT and AMP-ISD adjuvants as measured by ELISpot on splenocytes and ICS on lung lymphocytes and peripheral blood cells. Both CD8 and CD4 responses were detected.

### Example 5. Induction of T Cell Expansion Specific for Influenza

Amphiphilic adjuvants including amphiphilic CpG7909 and amphiphilic poly-dT were administered in combination with an Influenza A nucleoprotein antigen to elicit an immune response. The immune response elicited as a result of administration with AMP-CpG and AMP-dT were compared to their soluble counterparts and benchmarked against administering alum.

6 groups of 5 C57BL-6J mice each were administered a vaccine including 10 μg Influenza NP antigen and 5 nmol AMP-CpG, 10 μg Influenza NP antigen and 5 nmol soluble CpG, 10 μg Influenza NP antigen and 100 μg alhydrogel, 10 μg Influenza NP antigen and 5 nmol AMP-dT 50 nucleotides in length, 10 μg Influenza NP antigen and 5 nmol dT 50 nucleotides in length, or only AMP-CpG on Day 0 as described in Table 18. Immunizations were administered subcutaneously (SC) into the tail base of female B6 mice, bilaterally with 50 μL per side. A booster dose was given at roughly 2-week intervals on day 14 and 28. The SC injections ensured that the vaccine was optimally delivered into lymph nodes via natural lymph drainage, and bi-weekly injections were determined to be optimal in immune response generation.

The adjuvant stock solutions were resuspended in limulus amebocyte lysate (LAL) H₂O. The final injections were diluted with 1×Phosphate-buffered saline (PBS) such that AMP-CpG and soluble CpG had a concentration of 5 nmol/100 μL injection, AMP-dT and soluble dT had a concentration of 5 nmol/100 μL injection, and alum had a concentration of 100 μg/100 μL injection. The Influenza A H1N1 (A/Puerto Rico/8/34/Mount Sinai) Nucleoprotein (I116M) stock solutions were dissolved in LAL H₂O at a concentration of 0.25 mg/ml. The final injections were diluted with 1×PBS, resulting in a concentration of 10 μg/100 μL injection. The components used in the vaccines are described in Table 19.

TABLE 18
Summary of Influenza Vaccine Administration in Mice

| | | | | Dosing and Sample Collection | | | | | |
|---|---|---|---|---|---|---|---|---|---|
| | | Vaccine Components | | Day 0 | Day 14 | Day 14 | Day 21 | Day 28 | Day 35 |
| Group | Treatment Name | Antigen (10 ug) | Adjuvant (5 nmol) | Dose 1 | Read-out | Dose 2 | Read-out | Dose 3 | Read-out |
| 1 | AMP-CpG | Influenza NP | AMP-CpG 7909 | x | Tetramer | x | ICS on | x | ICS on |
| 2 | Sol CpG | Influenza NP | CpG 7909 | x | | x | blood | x | Lung and |
| 3 | Alum Vax | Influenza NP | Alhydrogel (100 ug) | x | | x | Tetramer | x | PBMCs |
| 4 | AMP-dT | Influenza NP | AMP-dT(50) | x | | x | | x | Spleen |
| 5 | Sol dT | Influenza NP | dT(50) | x | | x | | x | ELISpot |
| 6 | Adj Ctrl | — | AMP-CpG 7909 | x | | x | | x | |

TABLE 19
Vaccine Components

| Vaccine | Sequence | | |
|---|---|---|---|
| Components | or Cat# | Source | Lot # |
| Influenza | 11675- | Sino Bio | LC13MA2912 |
| Nucleoprotein | V08B | | |
| AMP-CpG7909 | 5′- | Avecia | 18-025-A |
| | (Diacyl | | |
| | lipid) | | |
| | tcgtcgt | | |
| | tttgtcg | | |
| | ttttgtc | | |
| | gtt-3′ | | |
| | (SEQ ID | | |
| | NO: 26) | | |
| CpG7909 | 5′-tcgtc | InvivoGen | 4110-70T |
| | gttttgtc | | |
| | gttttgtc | | |
| | gtt-3′ | | |
| | (SEQ ID | | |
| | NO: 26) | | |
| Alum | vac-alu- | InvivoGen | 1614532 |
| | 250 | | |
| dT-50-PS | 5′-dT-3′ | AXO | X-51176 K1-V4 |
| AMP-dT- | 5′-AMP- | AXO | X-51175 K1 |
| 50-PS | dT-3′ | | |

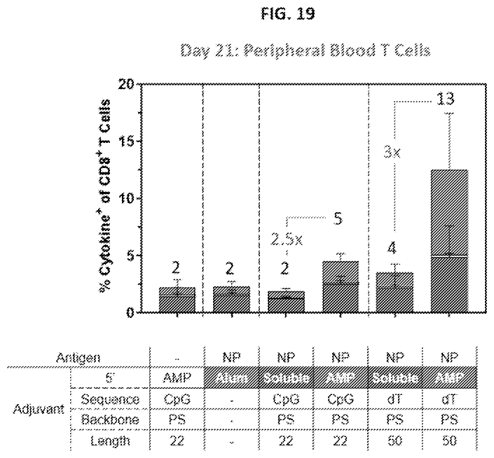

An ICS (Intracellular Stain) Assay for TNFα and IFNγ was performed on PBMCs 7 days after dosing. ICS was also performed on lung samples 7 days post dose 3. Cells were also surface stained for CD4, CD8 and CD3 using the antibodies described in Table 20. The ICS samples were activated overnight (in the presence of Brefeldin A and Monensin) with 2 μg/ml of the re-stimulation peptides PepMix H3N2 (PM-INFA_NP), PepMix H2N2 (BP21433), or Peptide SP-MHCI-0100 (Table 21). The results of the CS assay are summarized in FIG. 19.

TABLE 20
Antibodies used for ICS

| Antigen | Color | Source | Product # | Lot # |
|---|---|---|---|---|
| TNFα | FITC | BD | 554418 | 9123915 |
| IFNγ | PE | BD | 554412 | 9154769 |
| CD8a | APC | eBioscience | 17-0081-83 | 4321418 |
| CD4 | PE-Cy7 | Invitrogen | 25-0041-82 | 2123767 |
| CD3 | APC-Cy7 | BD | 560590 | 9179637 |
| LiveDead | Aqua | Invitrogen | L34966 | 1832692 |
| Brefeldin A | — | Invitrogen | 00-4506-51 | 1915300 |
| Monensin | — | BioLegend | 420701 | B297750 |

TABLE 21
Re-Stimulation Peptides

| Re-stimulation Peptides | Sequence | Source | Lot # |
|---|---|---|---|
| PepMix ™ Influenza A (NP | Pool of 122 overlapping | JPT | 41988CLü-01 |
| (H3N2)) | peptides (15mers with 11 aa | | |
| (PM-INFA_NP) | overlap) through | | |
| | Nucleoprotein (Swiss-Prot ID: | | |
| | O91743) of Influenza A virus | | |
| | (strain A/Kitakyushu/159/1993 | | |
| | H3N2) | | |
| PepMix ™ Influenza A | Pool of 122 overlapping | JPT | 230713BAR-1 |
| (NP/AnnArbor (H2N2) | peptides (15mers with 11 aa | | |
| (PM-INFA-NPH2N2) | overlap) through | | |
| | Nucleoprotein of Influenza A | | |
| | (H2N2) | | |
| NP peptide (H-2 Db) | ASNENMETM (SEQ ID | JPT | SP-35630FSe-23 |
| | NO: 27) | | |

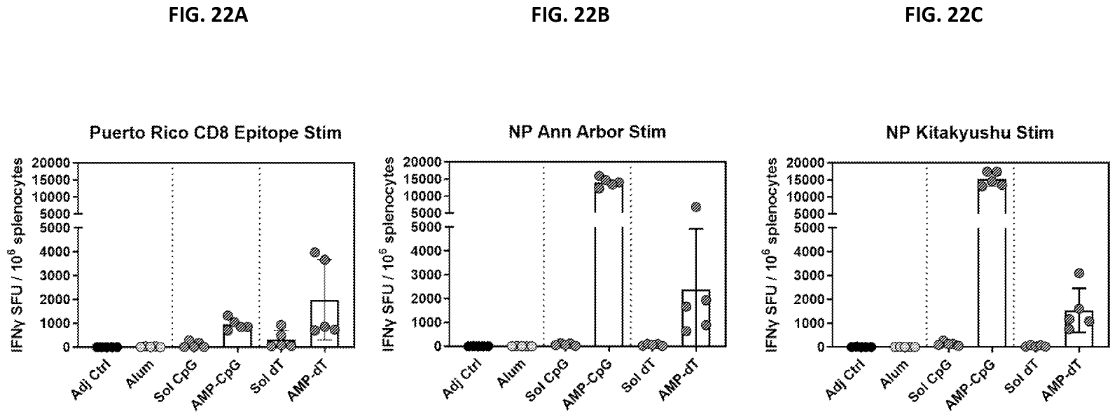

ELISpot analysis for IFNγ was performed on splenocytes after dose 3 administration. Splenocytes (0.125×10⁶ cells/well) were activated with 2 μg/ml PepMix (Table 21). The IFNγ plates were stimulated overnight (FIGS. 22A, 22B, and 22C).

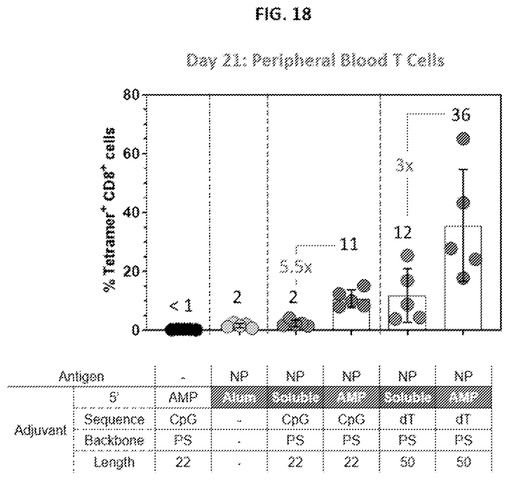

Tetramer analysis was performed 7 days post dosing using the H-2Db Influenza NP Tetramer-ASNENME™-PE 9 (SEQ ID NO:27) (FIG. 18).

The ICS, Tetramer and ELISpot data all showed strong immune responses with AMP-conjugated CpG as well as AMP-dT, which were significantly higher than their soluble counterparts or Alum. The responses measured by ELISpot and ICS also indicated that heterosubtypic immunity can be detected by immunization with a specific nucleoprotein variant plus AMP-adjuvant, and subsequent re-stimulating with the nucleoprotein from a different influenza serotype.

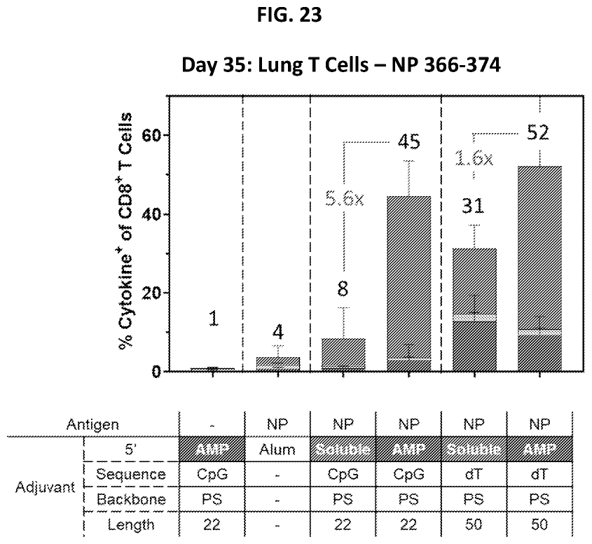

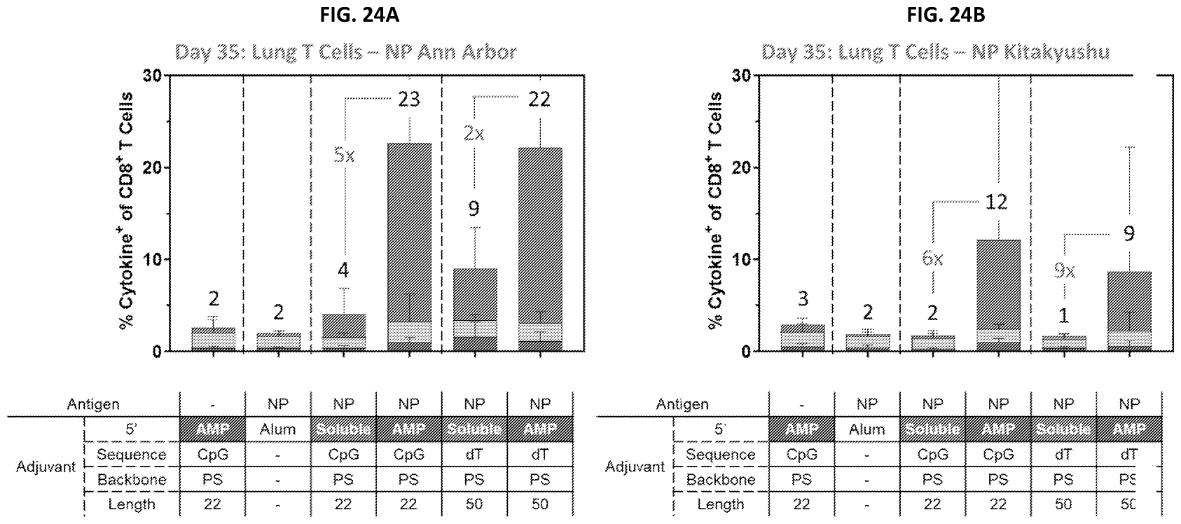

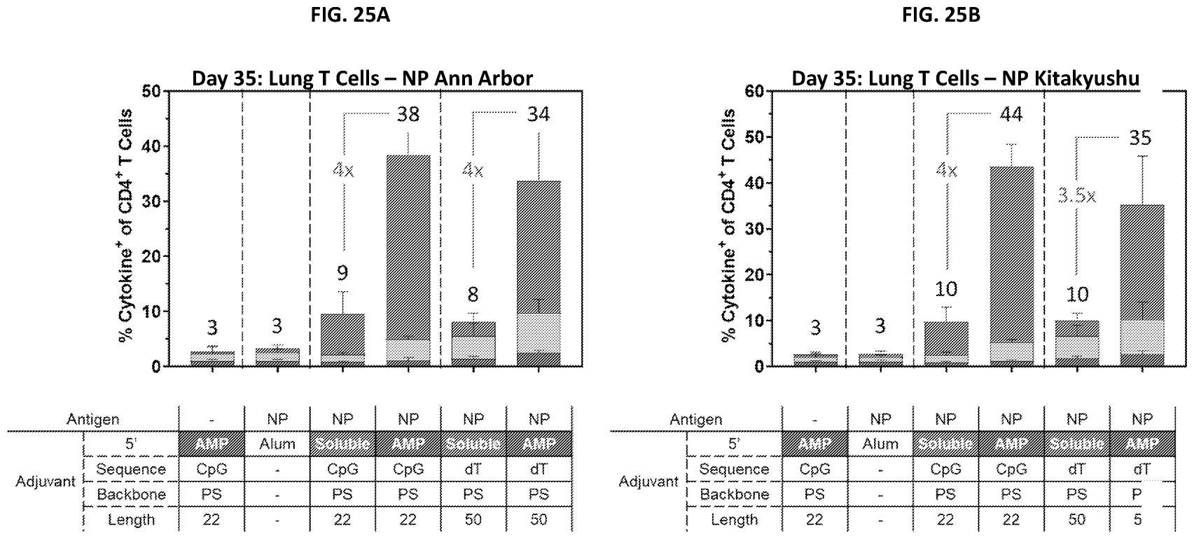

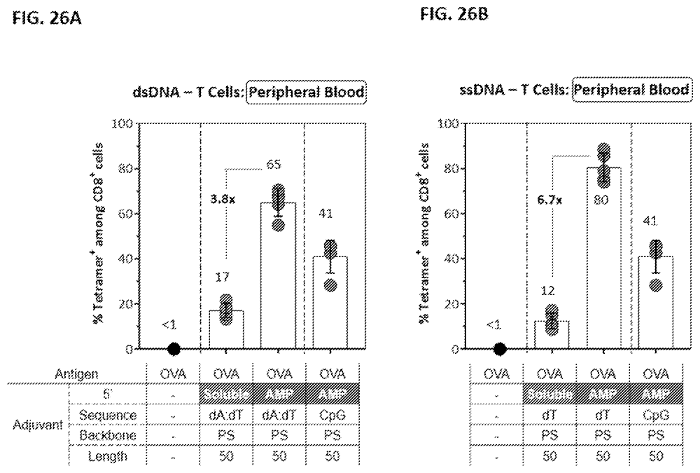

In the ICS and ELISpot assays, splenocytes that were restimulated with a CD8 epitope 9mer that matched the sequence of the nucleoprotein used to immunize the mice, elicited a strong CD8 T-cell response (FIGS. 22A to 22C and 23). When these samples were re-stimulated with a PepMix spanning the nucleoprotein sequence from two different serotypes of Influenza virus, Ann Arbor nucleoprotein and Kitakyushu nucleoprotein, strong CD4 and CD8 responses were observed (FIGS. 24A, 24B, 25A, and 25B), despite there being point mutations in the known CD8 epitopes. The known CD4 epitopes were not mutated in any of the stimulations. This indicates that despite single point mutations, the T-cell response can be maintained across different serotypes of influenza viruses.

### Example 6. Evaluating the Activity of Amphiphilic DNA Versus Naked DNA

The effects of amphiphilic DNA versus naked DNA on the immune response were determined. C57BL-6J mice were administered a vaccine having components including 5 nmol of double-stranded soluble hybridized dA and dT (dA:dT), double-stranded AMP dA:dT, soluble dT, or AMP dT, 165 μg (which is the mass equivalent of 5 nmol dA:dT) of naked double-stranded dA:dT (Invivogen), or 1 nmol of AMP-CpG7909 per injection, where the naked dA:dT was a poly(deoxyadenylic-deoxythymidylic) acid sodium salt (InvivoGen) including a repetitive synthetic double-stranded DNA sequence of poly(dA-dT):poly(dT-dA) and is a synthetic analog of B-DNA, as described in Table 22.

Immunizations were administered subcutaneously (SC) into the tail base of the mice, bilaterally with 50 μL per side. SC injections ensured that the vaccine was optimally delivered into lymph nodes via natural lymph drainage, and bi-weekly injections were determined to be optimal in immune response.

TABLE 22
Summary of Vaccine Administration in Mice

| | | | | Dosing and Sample Collection | | | |
|---|---|---|---|---|---|---|---|
| | | Vaccine Components | | Day 0 | Day 13 | Day 14 | Day 21 |
| Group | Treatment Name | Antigen (5 ug) | Adjuvant (5 nmol) | Dose 1 | Read-out | Dose 2 | Read-out |
| 1 | dA/dT-50-PS | OVA | 5′-dT50-3′ | x | ICS | x | ICS on |
| | | | 5′-dA50-3′ | | | | Lung and |
| 2 | AMP-dA/dT-50-PS | OVA | 5′-AMP-dT50-3′ | x | | x | PBMCs |
| | | | 5′-dA50-3′ | | | | Spleen |
| 3 | dT-50-PS | OVA | 5′-dT50-3′ | x | | x | ELISpot |
| 4 | AMP-dT-50-PS | OVA | 5′-AMP-dT50-3′ | x | | x | |
| 5 | naked dAdT | OVA | InvivoGen dAdT | x | | x | |
| | | | (165 ug) | | | | |
| 6 | AMP-CpG7909 | OVA | AMP-CpG7909 | x | | x | |
| | | | (1 nmol) | | | | |
| 7 | Mock (PBS) | OVA | — | x | | x | |

The adjuvant stock solutions were resuspended in limulus amebocyte lysate (LAL) H₂O, and the final injections were diluted with 1×Phosphate-buffered saline (PBS such that the (AMP-)dAdT and dT solution had a concentration of 5 nmol/100 μL injection, the AMP-CpG7909 had a concentration of 1 nmol/100 μL injection, and the naked dAdT had a concentration of 165 μg/100 μL injection. The ovalbumin protein stock solutions were made by dissolving ovalbumin in PBS to give a concentration of 2 mg/mL. The final injections of ovalbumin were diluted with 1×PBS result in a concentration of 5 μg/100 μL injection. The components of the vaccine are described in Table 23.

The immunizations were administered subcutaneously (SC) into the tail base of female B6 mice bilaterally in an amount of 50 μL per side. Booster doses was given at roughly 2-week intervals. The subcutaneous injections ensured that the vaccine was optimally delivered into the lymph nodes by way of natural lymph drainage. It was determined in previous mouse studies that bi-weekly injections were optimal in immune response generation.

TABLE 23
Vaccine Components

| Vaccine | Sequence | | |
|---|---|---|---|
| Components | or | Source | Lot # |
| OVA | Cat# | InvivoGen | EFP-38-04 |
| dA/dT- | 5′-dT-3′ | AXO | XD-15740 K2 |
| 50-PS | 5′-dA-3′ | | |
| AMP-dA/ | 5′-AMP- | AXO | XD-15739 K3 |
| dT-50-PS | dT-3′ | | |
| | 5′-dA-3′ | | |
| dT-50-PS | 5′-dT-3′ | AXO | X-51176 K1-V4 |
| AMP-dT- | 5′-AMP-dT-3′ | AXO | X-51175 K1 |
| 50-PS | | | |
| Naked | poly(dA-dT): | InvivoGen | 6250-42-03 |
| dAdT | poly(dT-dA) | | |
| AMP- | 5′-(Diacyl | Avecia | 18-025-A |
| CpG7909 | lipid) | | |
| | tcgtcg | | |
| | ttttgt | | |
| | cgtttt | | |
| | gtcgtt | | |
| | -3′ | | |
| | (SEQ ID | | |
| | NO: 26) | | |

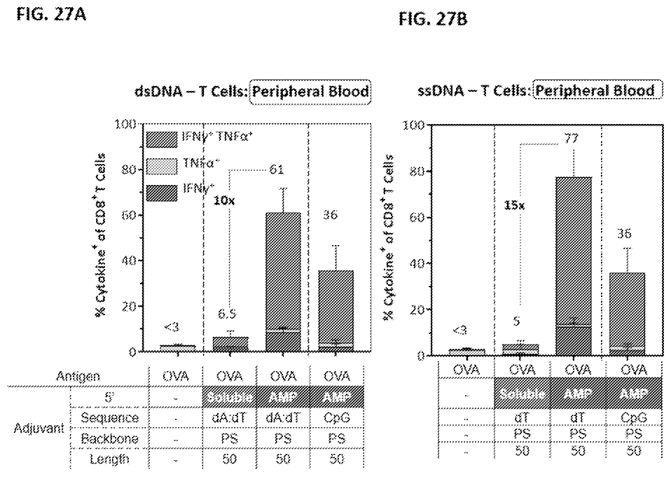

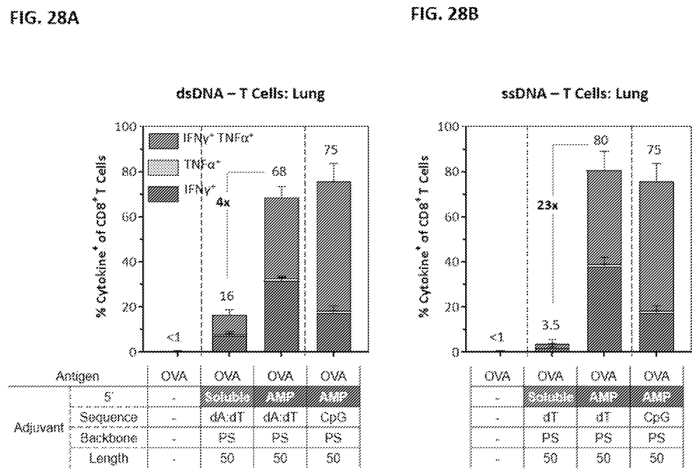

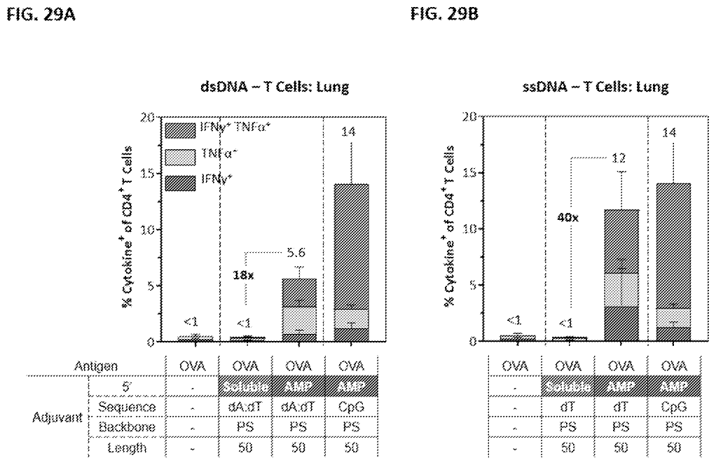

An ICS (Intracellular Stain) Assay for TNFα and IFNγ was performed on PBMCs 7 days after dosing 2. ICS was also performed on lung samples 7 days post dose 2 (FIGS. 21, 27A and 27B). ICS was also performed on lung samples 7 days post dose 2 (FIGS. 28A and 28B and FIGS. 29A and 29B). The cells were surface stained for CD4, CD8, and CD3. The antibodies described in Table 24 were used for the ICS assay.

TABLE 24
Antibodies used for ICS

| Antigen | Color | Source | Product # | Lot # |
|---|---|---|---|---|
| TNFα | FITC | BD | 554418 | 9123915 |
| IFNγ | PE | BD | 554412 | 9154769 |
| CD8a | APC | eBioscience | 17-0081-83 | 4321418 |
| CD4 | PE-Cy7 | Invitrogen | 25-0041-82 | 2123767 |
| CD3 | APC-Cy7 | BD | 560590 | 9179637 |
| LiveDead | Aqua | Invitrogen | L34966 | 1832692 |
| Brefeldin A | — | Invitrogen | 00-4506-51 | 1915300 |
| Monensin | — | BioLegend | 420701 | B297750 |

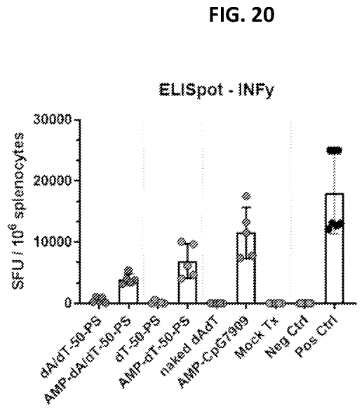

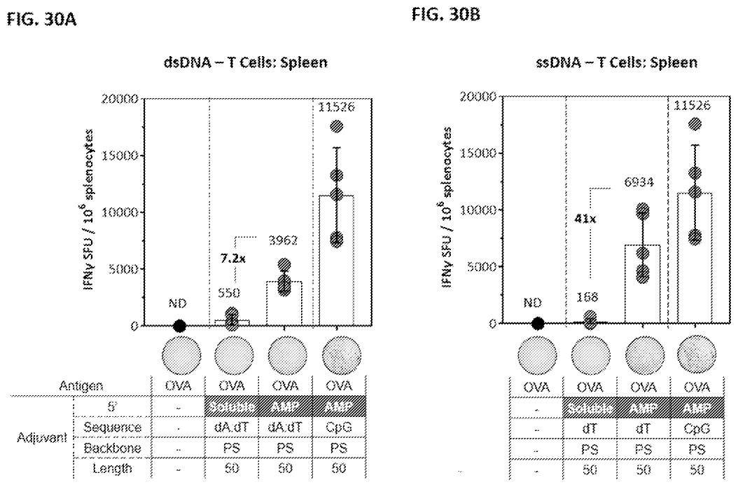

ELISpot analysis for IFNγ was performed on splenocytes after dose 2 administration (Splenocytes (0.1×10⁶ cells/well) were activated with 1 μg/ml PepTivator Ovalbumin Peptide Pool (Table 25), and the IFNγ plates were stimulated overnight (FIG. 20 and FIGS. 30A and 30B).

Tetramer analysis was performed 7 days post dosing using the T-Select I-Ab OVA 323-339 Tetramer-APC (ISQAVHAAHAEINEAGR) (SEQ ID NO: 28) and the iTAg Tetramer/PE-H-2Kb OVA (SIINFEKL) (SEQ ID NO:29) (FIGS. 26A and 26B).

The results showed that AMP-dAdT, AMP-dT and AMP-CpG all evoke a strong T-cell immune response compared to their soluble comparator adjuvants as measured by IFNγ ELISpot assay in splenocytes, Tetramer (MHCI-specific) stain of peripheral blood CD8⁺ cells, Intracellular stain of IFNy and TNFa cytokines in peripheral blood CD8⁺ and CD4⁺ T-cells, and Intracellular stain of IFNy and TNFa cytokines in lung CD8⁺ and CD4⁺ T-cells. Additionally, it was shown that the immune response induced by AMP-dT is equal to or greater than AMP-dAdT.

TABLE 25
Re-Stimulation Peptides

| Re-stimulation Peptides | Sequence | Source | Lot # |
|---|---|---|---|
| PepTivator Ovalbumin | 15mers spanning | Miltenyi | 5210909984 |
| | Ovalbumin, | | |
| | overlap 11aa | | |

### Example 7. Evaluating the Contribution of Length of Single-Stranded Amphiphilic DNA on Activity

This experiment performed to determine how length of the single-stranded poly-dT constructs affect immunogenicity.

15 groups of 5 C57BL-6J mice were administered a vaccine having components including 5 nmol of a SARS-CoV-2 antigen and 5 nmol single-stranded soluble or amphiphilic poly-dT. The poly-dT had a length of 10, 20, 30, 40, 50, 75, or 100 nucleotides, and all contained only a phosphorothioate backbone on Day 0, as described in Table 26.

TABLE 26
Summary of SARS-CoV-2 RBD Vaccine Administration in Mice

| | | | | Dosing and Sample Collection | | | |
|---|---|---|---|---|---|---|---|
| | | Vaccine Components | | Day 0 | Day 13 | Day 14 | Day 21 |
| Group | Treatment Name | Antigen (5 ug) | Adjuvant (5 nmol) | Dose 1 | Read-out | Dose 2 | Read-out |
| 1 | dT-10-PS | GenScript RBD | 5′-sdT10-3′ | x | ICS/ | x | ICS on |
| 2 | dT-20-PS | GenScript RBD | 5′-sdT20-3′ | x | Tetramer | x | Lung and |
| 3 | dT-30-PS | GenScript RBD | 5′-sdT30-3′ | x | | x | PBMCs |
| 4 | dT-40-PS | GenScript RBD | 5′-sdT40-3′ | x | | x | Spleen |
| 5 | dT-50-PS | GenScript RBD | 5′-sdT50-3′ | x | | x | ELISpot |
| 6 | dT-75-PS | GenScript RBD | 5′-sdT75-3′ | x | | x | |
| 7 | dT-100-PS | GenScript RBD | 5′-sdT100-3′ | x | | x | |
| 8 | AMP-dT-10-PS | GenScript RBD | 5′-AMP-dT10-3′ | x | | x | |
| 9 | AMP-dT-20-PS | GenScript RBD | 5′-AMP-dT20-3′ | x | | x | |
| 10 | AMP-dT-30-PS | GenScript RBD | 5′-AMP-dT30-3′ | x | | x | |
| 11 | AMP-dT-40-PS | GenScript RBD | 5′-AMP-dT40-3′ | x | | x | |
| 12 | AMP-dT-50-PS | GenScript RBD | 5′-AMP-dT50-3′ | x | | x | |
| 13 | AMP-dT-75-PS | GenScript RBD | 5′-AMP-dT75-3′ | x | | x | |
| 14 | AMP-dT-100-PS | GenScript RBD | 5′-AMP-dT100-3′ | x | | x | |
| 15 | Mock | — | — | x | | x | |

The adjuvant stock solutions were resuspended in limulus amebocyte lysate (LAL) H₂O, and the final injections were diluted with 1×Phosphate-buffered saline (PBS) such that the (AMP-)dT had a concentration of 5 nmol/100 μL injection. The SARS-CoV2 Spike 1 RBD protein stock solutions were made by dissolving in PBS at a concentration of 0.88 mg/mL, and the final injections were diluted with 1×PBS to have a concentration of 5 μg/100 μL injection. The components of the vaccine are described in Table 27.

The immunizations were administered subcutaneously (SC) into the tail base of female B6 mice bilaterally in an amount of 50 μL per side. Booster doses was given at roughly 2-week intervals. The subcutaneous injections ensured that the vaccine was optimally delivered into the lymph nodes by way of natural lymph drainage. It was determined in previous mouse studies that bi-weekly injections were optimal in immune response generation.

TABLE 27
Vaccine Components

| Vaccine | | | |
|---|---|---|---|
| Components | Sequence or Cat# | Source | Lot # |
| SARS-CoV2 | Z03483 | GenScript | B200931 |
| RBD, His | | | |
| dT10 | 5′-sdT₁₀-33′ | AXO | X-97920 K1 |
| dT20 | 5′-sdT₂₀-33′ | AXO | X-97921 K1 |
| dT30 | 5′-sdT₃₀-33′ | AXO | X-88284 K1-V2 |
| dT40 | 5′-sdT₄₀-33′ | AXO | X-88286 K1-V2 |
| dT50 | 5′-sdT₅₀-33′ | AXO | X-51175 K6-V2 |
| dT75 | 5′-sdT₇₅-33′ | AXO | X-88288 K2 |
| dT100 | 5′-sdT₁₀₀-33′ | AXO | X-88290 K2 |
| AMP-dT10 | 5′-AMP-sdT₁₀-33′ | AXO | X-97922 K1 |
| AMP-dT20 | 5′-AMP-sdT₂₀-33′ | AXO | X-97923 K1 |
| AMP-dT30 | 5′-AMP-sdT₃₀-33′ | AXO | X-88292 K1-V2 |
| AMP-dT40 | 5′-AMP-sdT₄₀-33′ | AXO | X-88293 K1-V2 |
| AMP-dT50 | 5′-AMP-sdT₅₀-33′ | AXO | X-51176 K1-V5 |
| AMP-dT75 | 5′-AMP-sdT₇₅-33′ | AXO | X-88294 K1-V2 |
| AMP-dT100 | 5′-AMP-sdT₁₀₀-33′ | AXO | X-88295 K1-V2 |

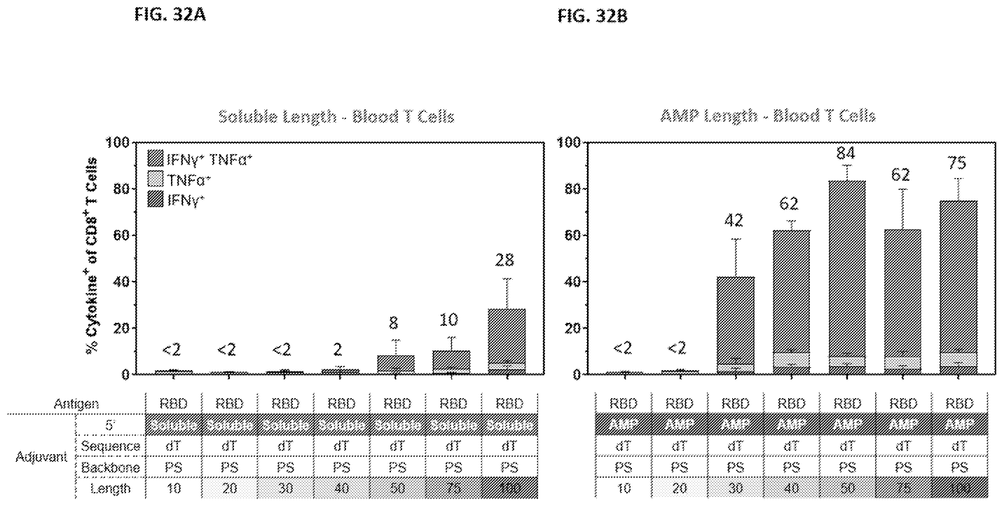

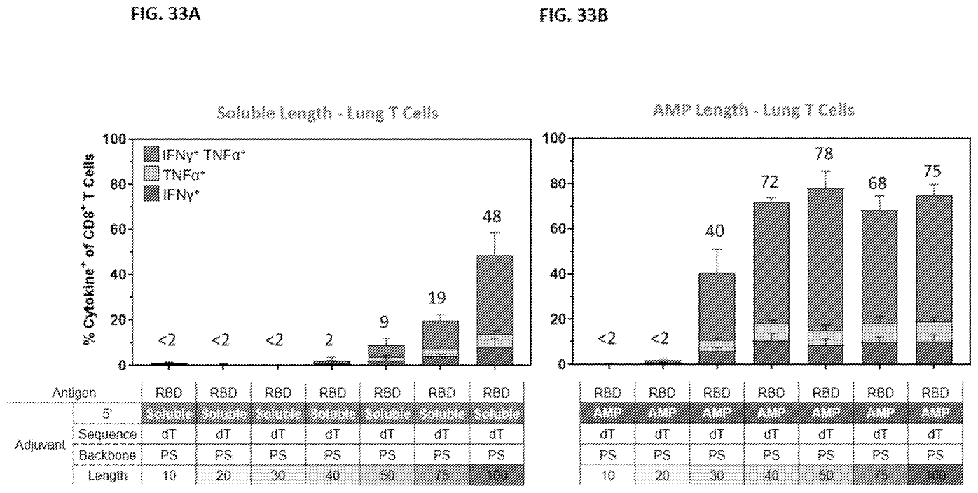

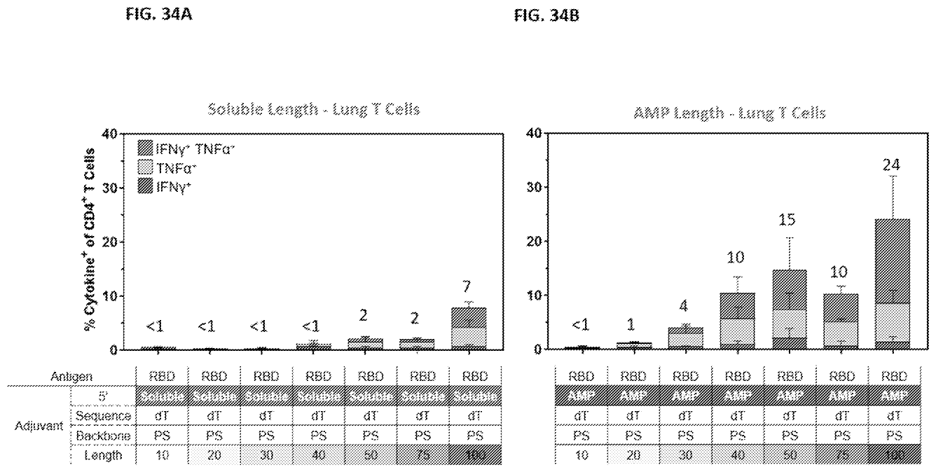

An ICS (Intracellular Stain) Assay for TNFα and IFNγ was performed on PBMCs 7 days after dosing (FIGS. 32A and 32B). ICS was also performed on lung samples 7 days post dose 2 (FIGS. 33A and 33B and FIGS. 34A and 34B). The cells were surface stained for CD4, CD8, and CD3. The antibodies described in Table 28 were used for the ICS assay. ICS samples were activated overnight (in the presence of Brefeldin A and Monensin) with 1 μg/mL of SARS-CoV-2 Spike Glycoprotein Peptide Pool Mix [315 peptides each at 1 μg/well](Table 29).

TABLE 28
Antibodies used for ICS

| Antigen | Color | Source | Product # | Lot # |
|---|---|---|---|---|
| TNFα | FITC | BD | 554418 | 9123915 |
| IFNγ | PE | BD | 554412 | 9154769 |
| CD8a | APC | eBioscience | 17-0081-83 | 4321418 |
| CD4 | PE-Cy7 | Invitrogen | 25-0041-82 | 2123767 |
| CD3 | APC-Cy7 | BD | 560590 | 9179637 |
| LiveDead | Aqua | Invitrogen | L34966 | 1832692 |
| Brefeldin A | — | Invitrogen | 00-4506-51 | 1915300 |
| Monensin | — | BioLegend | 420701 | B297750 |

TABLE 29
Re-Stimulation Peptides

| Re-stimulation | | | |
|---|---|---|---|
| Peptides | Sequence | Source | Lot # |
| SARS-CoOV-2 Spike | 315 15mers spanning | GenScript | E5868620K |
| Glycoprotein | Spike Protein | | |
| Peptide Pool Mix | Sequence, overlap 11aa | | |

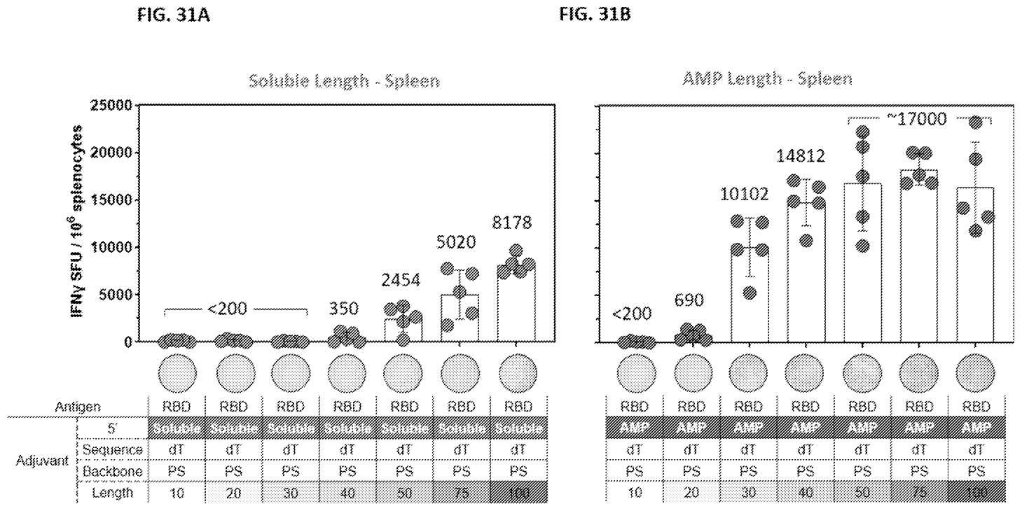

ELISpot analysis for IFNγ was performed on splenocytes after dose 2 administration (FIGS. 31A and 31B). Splenocytes (0.1×10⁶ cells/well) were activated with 1 μg/ml SARS-CoV-2 Spike Glycoprotein Peptide Pool Mix (Table 29). The IFNγ plates were stimulated overnight.

Tetramer analysis was performed 7 days post dosing using the H-2Kb CoV2 RBD Tetramer-VNFNFNGL-PE (SEQ ID NO:25) (NIH 53309).

A SARS-CoV2 specific serum ELISA (enzyme-linked immunosorbent assay) was performed on mouse serum 7 days after each dose, to detect any RBD-specific antibody response. The samples were collected by centrifuging whole blood using Ser-gel tubes (NC9436363, Fisher Scientific). The serum was either used fresh or stored at −80° C. until used. 96-well plates were coated with 200 ng/100 μL (2 μg/mL) of CoV2 RBD protein (Z03483, GenScript) overnight at 4° C. Then plates were pre-blocked with 2% BSA for 2 h at RT. Mouse serum was diluted to 1:20 and then serially diluted (1:5→8 concentrations) in a dummy plate. ELISA plates were washed once with ELISA washing buffer (BioLegend 4211601). Samples were transferred to the ELISA plate and incubated for 2 h at RT. Plates were washed 4 times with washing buffer. For serum antibody detection the secondary HRP-conjugated antibodies in Table 30 were used at 1:2000 in PBS+ and incubated for 1 h at RT. Plates were washed 4 times with washing buffer. The reaction was visualized by addition of substrate 3,3′,5,5′-Tetramethylbenzidine (TMB) for 10 min at RT and stopped by H2SO4 (1 N). The absorbance at 450 nm was measured by an ELISA plate reader.

TABLE 30
Secondary HRP-conjugated antibodies

| Antigen | Source | Product # | Lot # |
|---|---|---|---|
| IgG | Jackson | 315-035-046 | 147406 |
| | Immunoresearch | | |
| IgG1 | Jackson | 115-035-205 | 148255 |
| | Immunoresearch | | |
| IgG2c | Jackson | 115-035-2081 | 146880 |
| | Immunoresearch | | |

The results from this experiment indicated that AMP-conjugation of dT DNA improves its immunogenicity. The AMP-conjugated dT length variants were able to illicit a strong immune response down to a base-length of 30 nucleic acids. 10 and 20 bases in the AMP-dT-construct did not illicit an immune response. When unconjugated soluble dT-DNA was used, responses were unobservable up to a length of 40 bases. Immune response increased as soluble dT length increased. However, dT100 evoked immune responses that were equal to or less than the responses of AMP-dT30. These data may indicate a length cut-off in the recognition by pattern recognition receptors (PRRs), that do not interact with DNA shorter than 30 bases.

### Example 8. Evaluating Amphiphilic DNA as an Adjuvants Against Other Adjuvants

This experiment was performed to compare the AMP-versions of DNA-adjuvants against 5 standard adjuvants that are commonly used in commercially available vaccines. The antigen ovalbumin (OVA) was used to allow broad comparison with other results in the literature.

9 groups of 5 C57BL-6J mice were administered a vaccine having components including 5 nmol of a ovalbumin antigen and amphiphilic double-stranded dA hybridized to dT (dA:dT), amphiphilic poly-dT, amphiphilic CpG, alum, IFA, MF59, AS03, or AS04, on Day 0 as described om Table 31.

TABLE 31
Summary of Ovalbumin Vaccine Administration in Mice

| | | | | Dosing and Sample Collection | | | |
|---|---|---|---|---|---|---|---|
| | | Vaccine Components | | Day 0 | Day 13 | Day 14 | Day 21 |
| Group | Treatment Name | Antigen (5 ug) | Adjuvant | Dose 1 | Read-out | Dose 2 | Read-out |
| 1 | AMP-dAdT | OVA | 5′-AMP-dT50-3′ | x | ICS/ | x | ICS on |
| | | | 5′-dA50-3′ (5 nmol) | | Tetramer | | Lung and |
| 2 | AMP-dT | OVA | 5′-AMP-dT50-3′ (5 nmol) | x | | x | PBMCs |
| 3 | AMP-CpG | OVA | AMP-CpG7909 (1 nmol) | x | | x | Spleen |
| 4 | AS03 | OVA | AS03 | x | | x | ELISpot |
| 5 | AS04 | OVA | AS04 | x | | x | |
| 6 | MF59 | OVA | MF59 | x | | x | |
| 7 | IFA | OVA | IFA | x | | x | |
| 8 | Alum | OVA | Alum | x | | x | |
| 9 | Mock | OVA | — | x | | x | |

The adjuvant stock solutions were resuspended in limulus amebocyte lysate (LAL) H₂O, and the final injections were diluted with 1×Phosphate-buffered saline (PBS) such that AMP-dAdT and AMP-dT had a concentration of 5 nmol/100 μL injection, AMP-CpG7909 had a concentration of 1 nmol/100 μL injection, alum had a concentration of 10 μg/100 μL injection, and ASO4 had a concentration of 10 μg/100 μL injection. The monophosphoryl lipid A (MPLA) was derived from Salmonella enterica LPS. The other adjuvant solutions were supplied as 2× stock solutions and their concentrations were diluted 1:2 with PBS in the final dose. The IFA was a water-in-oil emulsion of 15% Mannide Monooleate and 85% Paraffin oil. The AS03 included DL-α-tocopherol (5% v/v) in squalene oil 5% (v/v) and Tween80® (10.8% v/v) in phosphate-buffered saline (pH 6.8). The MF56 included Sorbitan trioleate (0.5% w/v) in squalene oil (5% v/v) and Tween80® (0.5% v/v) in sodium citrate buffer (10 mM, pH 6.5). The ovalbumin protein stock solutions were dissolved in PBS at a concentration of 2 mg/mL, and the final injections were diluted with 1×PBS to a concentration of 5 μg/100 μL injection. The components of the vaccines administered are described in Table 32.

The immunizations were administered subcutaneously (SC) into the tail base of female B6 mice bilaterally in an amount of 50 μL per side. Booster doses was given at roughly 2-week intervals. The subcutaneous injections ensured that the vaccine was optimally delivered into the lymph nodes by way of natural lymph drainage. It was determined in previous mouse studies that bi-weekly injections were optimal in immune response generation.

TABLE 32
Vaccine Components

| Vaccine | | | |
|---|---|---|---|
| Components | Sequence | Source | Lot # |
| OVA | or Cat# | InvivoGen | EFP-38-04 |
| AMP-dT50 | 5′-AMP- | AXO | X-51175-K8 |
| | sdT₅₀-3′ | | |
| AMP-dAdT50 | 5′-AMP- | AXO | XD-15739-K5 |
| | dT50-3′ | | |
| | 5′-dA50-3′ | | |
| AMP-CpG7909 | 5′-(Diacyl | Avecia | 18-025-A |
| | lipid) | | |
| | tcgtcgt | | |
| | tttgtcg | | |
| | ttttgtc | | |
| | gtt-3′ | | |
| | (SEQ ID | | |
| | NO: 26) | | |
| Alum | | InvivoGen | 1614532 |
| MF59 | vac-adx-10 | InvivoGen | 5805-43-02 |
| AS03 | vac-as03-10 | InvivoGen | 10253-42-02 |
| AS04 | vac-mpla | InvivoGen | 5815-42-02 |
| IFA | vac-ifa-10 | InvivoGen | IFA-41-01 |

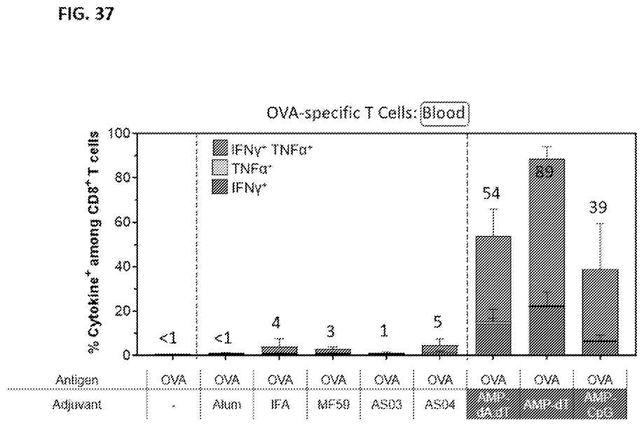

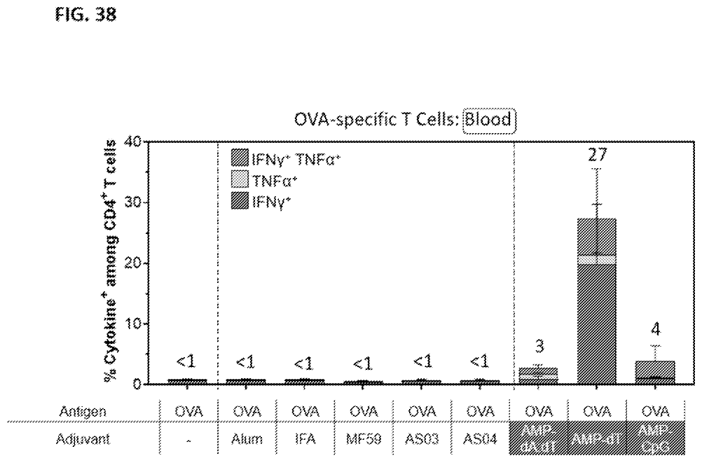

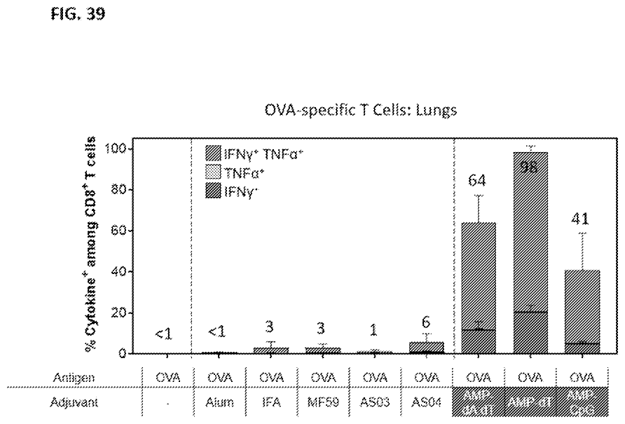

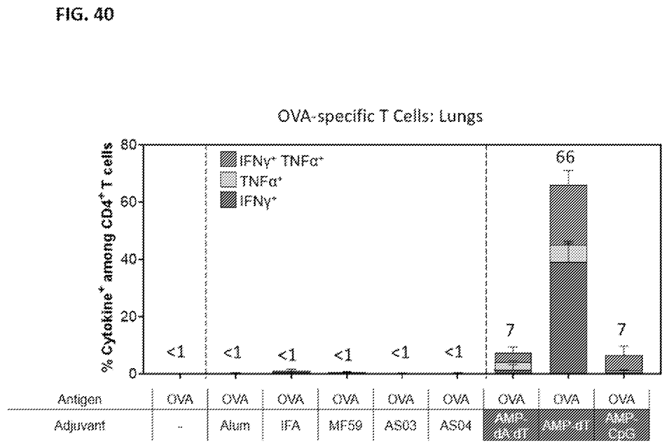

An ICS (Intracellular Stain) Assay for TNFα and IFNγ was performed on PBMCs 7 days after dosing (FIGS. 37 and 38). ICS was also performed on lung samples 7 days post dose 2 (FIGS. 39 and 40). The cells were surface stained for CD4, CD8, and CD3. The antibodies described in Table 33 were used for the ICS assay. ICS samples were activated overnight (in the presence of Brefeldin A and Monensin) with 1 μg/mL of PepTivator Ovalbumin Peptide Pool Mix (Table 34).

TABLE 33
Antibodies used for ICS

| Antigen | Color | Source | Product # | Lot # |
|---|---|---|---|---|
| TNFα | FITC | BD | 554418 | 9123915 |
| IFNγ | PE | BD | 554412 | 9154769 |
| CD8a | APC | eBioscience | 17-0081-83 | 4321418 |
| CD4 | PE-Cy7 | Invitrogen | 25-0041-82 | 2123767 |
| CD3 | APC-Cy7 | BD | 560590 | 9179637 |
| LiveDead | Aqua | Invitrogen | L34966 | 1832692 |
| Brefeldin A | — | Invitrogen | 00-4506-51 | 1915300 |
| Monensin | — | BioLegend | 420701 | B297750 |

TABLE 14
Re-Stimulation Peptides

| Re-stimulation | | | |
|---|---|---|---|
| Peptides | Sequence | Source | Lot # |
| PepTivator | 15mers spanning | Miltenyi | 5210909984 |
| Ovalbumin | Ovalbumin, | | |
| | overlap 11aa | | |

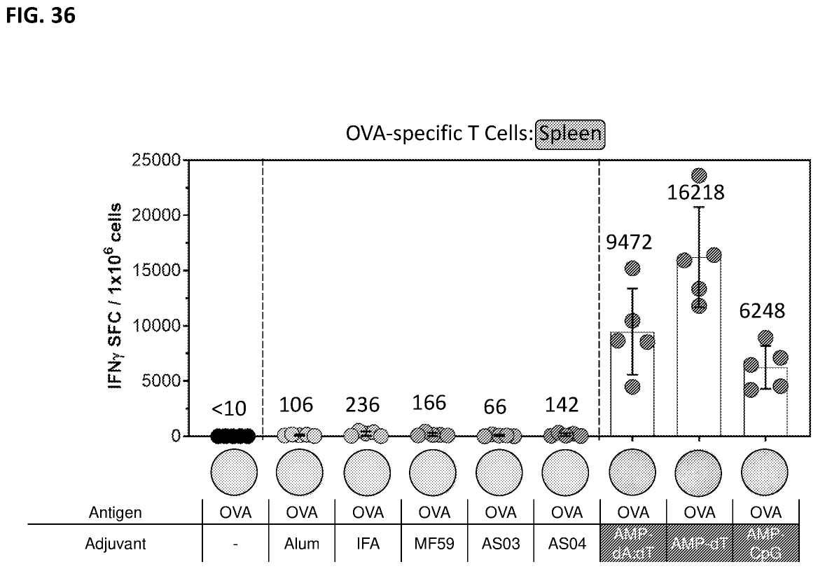

ELISpot analysis for IFNγ was performed on splenocytes after dose 2 administration (FIG. 36). Splenocytes (0.1×10⁶ cells/well) were activated with 1 μg/ml PepTivator Ovalbumin Mix (Table 34). The IFNγ plates were stimulated overnight.

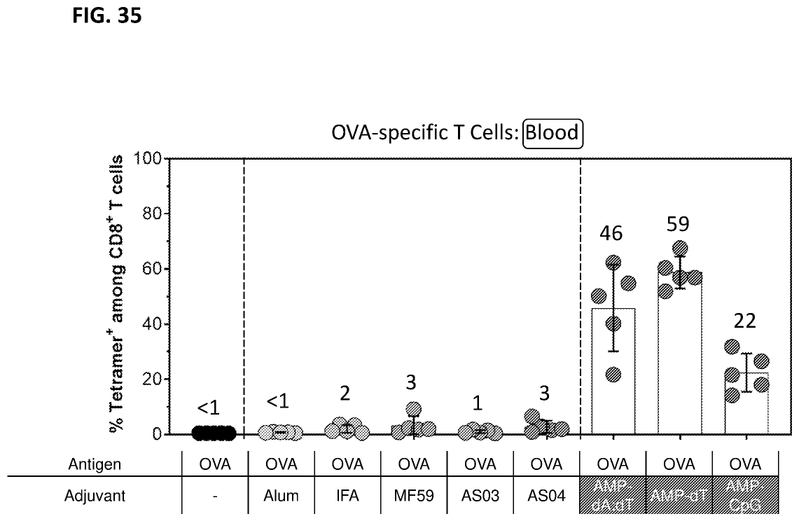

Tetramer analysis was performed 7 days post dosing using the T-Select I-Ab OVA 323-339 Tetramer-APC (ISQAVHAAHAEINEAGR) (SEQ ID NO:28) and the iTAg Tetramer/PE-H-2Kb OVA (SIINFEKL) (SEQ ID NO:29) (FIG. 35).

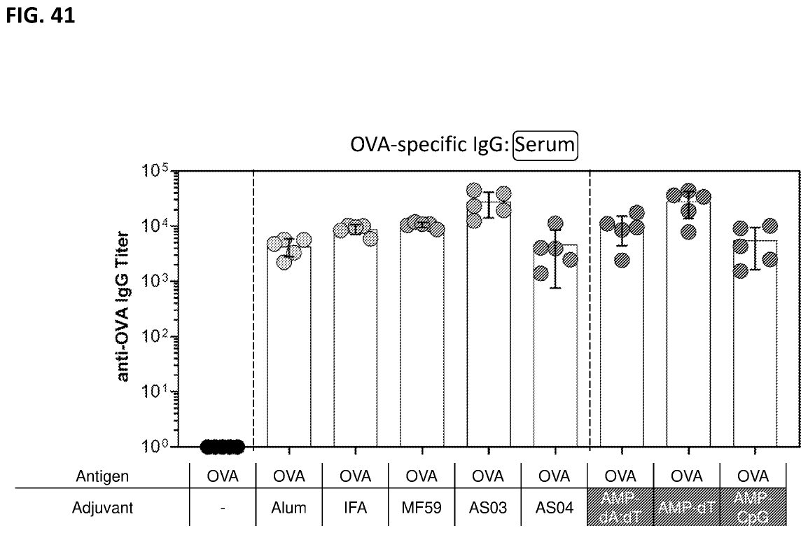

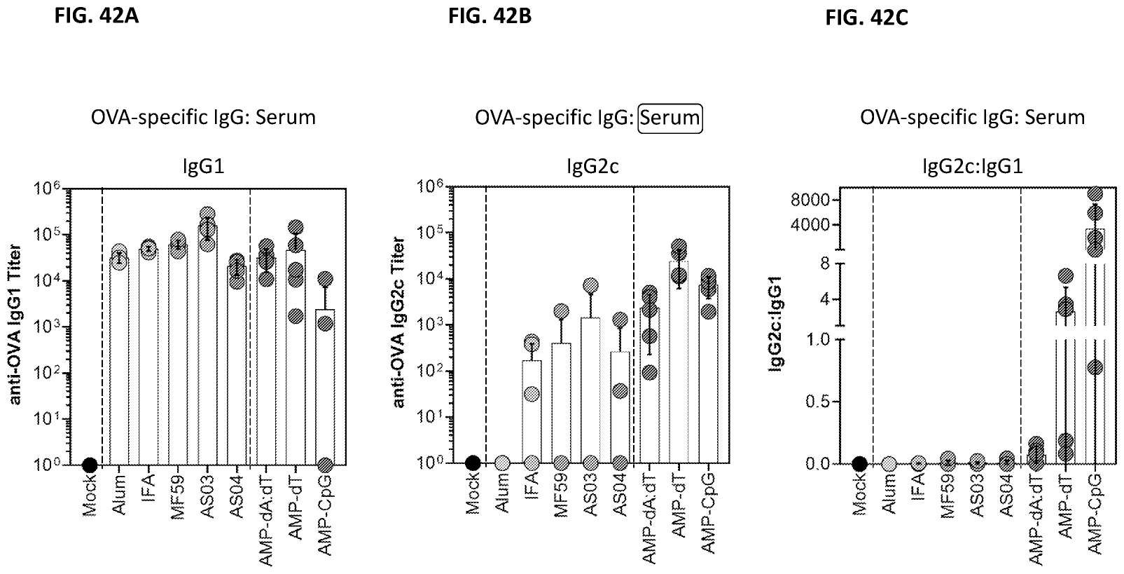

An ovalbumin specific serum ELISA (enzyme-linked immunosorbent assay) was performed on mouse serum 7 days after each dose, to detect any ovalbumin specific antibody response. The samples were collected by centrifuging whole blood using Ser-gel tubes (NC9436363, Fisher Scientific). The serum was either used fresh or stored at −80° C. until used. 96-well plates were coated with 200 ng/100 μL (2 μg/mL) of ovalbumin overnight at 4° C. Then plates were pre-blocked with 2% BSA for 2 h at RT. Mouse serum was diluted to 1:20 and then serially diluted (1:5→8 concentrations) in a dummy plate. ELISA plates were washed once with ELISA washing buffer (BioLegend 4211601). Samples were transferred to the ELISA plate and incubated for 2 h at RT. Plates were washed 4 times with washing buffer. For serum antibody detection the secondary HRP-conjugated antibodies in Table 35 were used at 1:2000 in PBS+ and incubated for 1 h at RT. Plates were washed 4 times with washing buffer. The reaction was visualized by addition of substrate 3,3′,5,5′-Tetramethylbenzidine (TMB) for 10 min at RT and stopped by H2SO4 (1 N). The absorbance at 450 nm was measured by an ELISA plate reader. The results of these studies are summarized in FIGS. 41 and 42A-42C.

TABLE 35
Secondary HRP-conjugated antibodies

| Antigen | Source | Product # | Lot # |
|---|---|---|---|
| IgG | Jackson | 315-035-046 | 147406 |
| | Immunoresearch | | |
| IgG1 | Jackson | 115-035-205 | 148255 |
| | Immunoresearch | | |
| IgG2b | Jackson | 115-035-207 | 145800 |
| | Immunoresearch | | |

The results of this experiment indicated that the T-cell immune response generated by AMP-adjuvants was robust. In comparison, every industry standard comparator failed to illicit T-cell responses. These assays included ELISpot analysis of splenocytes, intracellular stain and flow cytometry of peripheral blood and lung CD4⁺ and CD8⁺ T-cells and tetramer analysis. IgG antibody titers in all groups were robust and comparable in magnitude. However, when breaking down the IgG isotype response, it was observed that comparator adjuvants had a skewed response towards Th2-associated antibodies (IgG1) and lower to no Th1-associated antibodies (IgG2c). AMP-conjugated adjuvants in contrast showed a strong skewing towards Th1 responses.

### Example 9. Induction of T Cell Expansion Specific for SARS-CoV-2 in Non-Human Primates

This experiment was performed to test the SARS-CoV2 amphiphilic vaccine in nonhuman primates (NHP). The adjuvants, amphiphilic CpG7909 (AMP-CpG7909) and the amphiphilic poly-dT having 50 nucleotides in length (AMP-dT50) were examined in combination with the Spike RBD and Spike protein antigens as described below. The objective of the study was to determine toxicity and tolerability of the AMP-vaccines in primates, as well as determining optimal vaccine composition and concentrations.

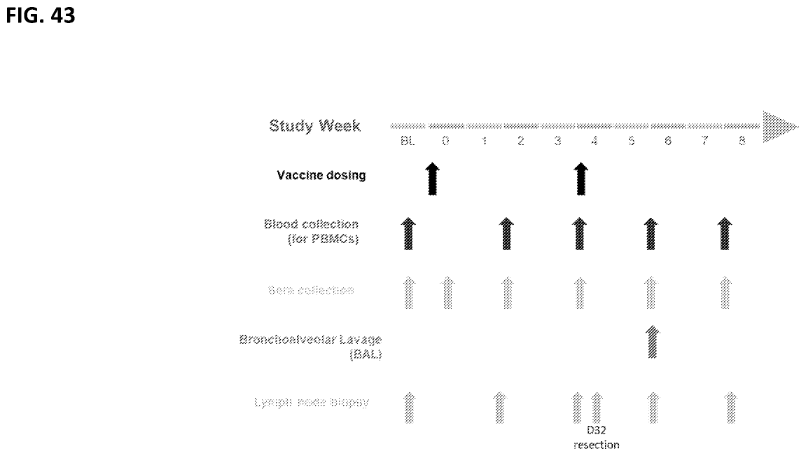

9 groups of 2-6 Rhesus Macaque monkeys were administered an adjuvant and a SARS-CoV-2 RBD or Spike antigen. Each group was administered an adjuvant of amphiphilic CpG7909 or amphiphilic poly-dT having 50 nucleotides and a SARS-CoV-2 Spike antigen including the SARS-CoV-2 RBD, the Delta variant RBD, the Beta variant RBD, or the SARS-CoV-2 Spike protein antigen as described in Table 36, and the vaccine components are described in Table 37. Groups 3-5 were designed to escalate the dose of CpG. Group 6 was designed to test AMP-CpG with SARS-CoV2 variants of concern. Group 7 was designed to test AMP-dT. Group 8 was designed to test AMP-CpG and Spike protein. The vaccines where dosed and samples were collected as described in FIG. 43.

TABLE 36
Study Groups

| Group | Vaccine | # of animals |
|---|---|---|
| 3 | 3000 μg AMP-CpG + 140 μg Genscript RBD | 6 |
| 4 | 5000 μg AMP-CpG + 140 μg Genescript RBD | 3 |
| 5 | 10000 μg AMP-CpG + 140 μg Genescript RBD | 3 |
| 6 | 5000 μg AMP-CpG + 140 μg Delta RBD + | 3 |
| | 140 μg Beta RBD | |
| 7 | 5000 μg AMP-dT + 140 μg Genescript RBD | 3 |
| 8 | 5000 μg AMP-CpG + 140 μg Acro Spike protein | 2 |

The adjuvant stock solutions of AMP-CpG7909 and AMP-dT50 were resuspended in limulus amebocyte lysate (LAL) H₂O. Final injections were diluted with 1×Phosphate-buffered saline (PBS). The SARS-CoV2 Spike 51 RBD and Spike protein stock solutions were dissolved in PBS. Final injections were diluted with 1×PBS. The immunizations were administered subcutaneously (SC) into each limb of the NHPs. A booster dose was given 4 weeks after first injection.

TABLE 37
Vaccine Components

| Vaccine | Sequence | | |
|---|---|---|---|
| Components | or Cat# | Source | Lot # |
| SARS-CoV2 | Z03483 | GenScript | B2009021 |
| RBD WT | | | |
| SARS-CoV2 | Z03537 | GenScript | B2101019 |
| RBD Beta | | | |
| SARS-CoV2 | Z03613 | GenScript | T2105057 |
| RBD Delta | | | |
| SARS-CoV2 | SPN-C52H9 | Acro | |
| Spike WT | 5′- | Avecia | 18-025-A |
| AMP-CpG7909 | (Diacyl | | |
| | lipid) | | |
| | tcg | | |
| | tcg ttt | | |
| | tgt cgt | | |
| | ttt gtc | | |
| | gtt-3′ | | |
| | (SEQ ID | | |
| | NO: 26) | | |
| AMP-dT50 | 5′-AMP- | AXO Labs | XD-15739K4 |
| | dT50-3′ | | |

After receipt of the collected serum, it was stored at −80° C. until used. After thawing, the serum was heat-inactivated at 56° C. for 30 minutes, aliquoted, and either refrozen or used immediately. The working aliquots were stored at 4° C. for further use for a maximum of 1 week.

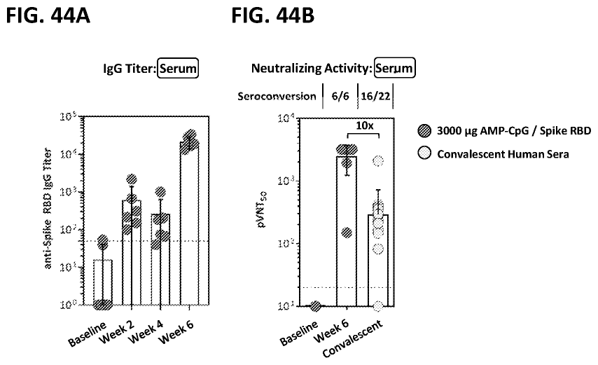

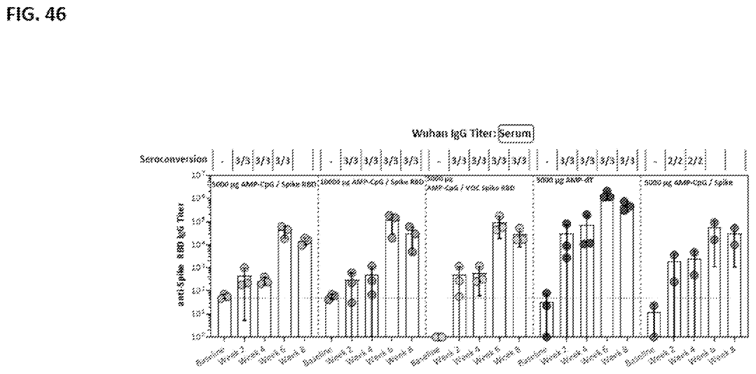

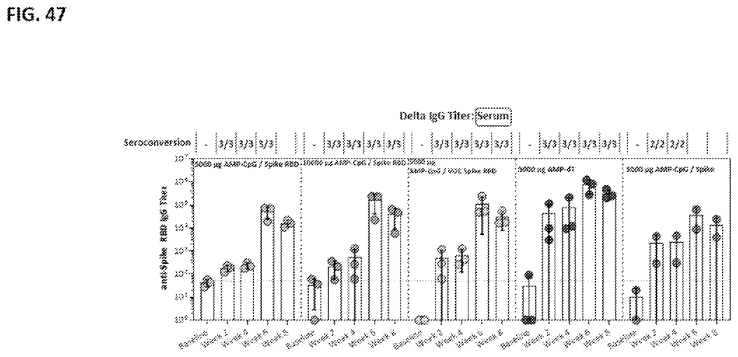

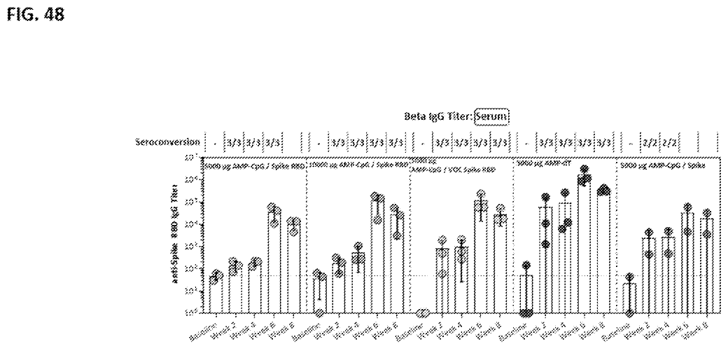

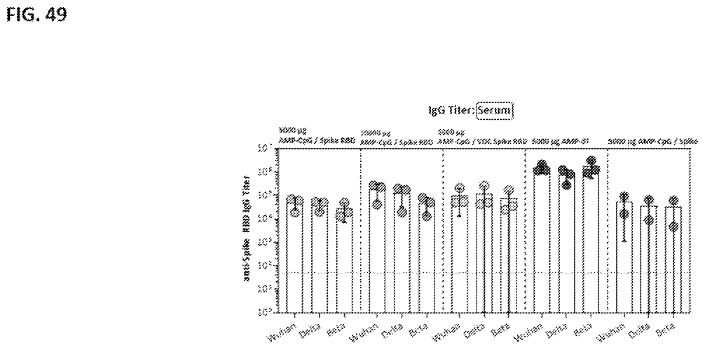

96-well flat-bottom Maxisorp plates were coated with 100 ng/well (1 μg/ml) of antigen, where the antigen was RBD WT (GenScript Z03483), RBD Beta (GenScript Z03537), or RBD Delta (GenScript Z03613). The serum was serially diluted in a fresh untreated 96-well flat-bottom plate by a 1:20 initial dilution in PBS with subsequent 1:4 serial dilution in PBS. The coated ELISA plates were washed once with PBS and pre-blocked with casein blocking solution (Thermo Fisher 37582) for 1-2 h at RT. The serially diluted serum samples were transferred to the coated ELISA plate and incubated for 2 h at RT. The plates were washed 3 times with washing buffer (BioLegend 4211601). For serum antibody detection, an HRP-conjugated secondary antibody was used diluted in PBS+(Thermo Fisher (Cat #PAl84631)) at 1:2000 incubated for 1 h at RT. The plates were washed 3 times with washing buffer. The reaction was visualized by addition of substrate 3,3′,5,5′-Tetramethylbenzidine (TMB) for 15 m at RT and stopped by H2SO4 (1 N). The absorbance was measured at 450 nm by an ELISA plate reader. The results of the ELISA assays are summarized in FIG. 44A and FIG. 46-FIG. 49.

The collected PBMCs were received frozen. The cells were thawed using thawing media (RPMI+50 IU/mL Benzonase [Millipore Sigma, Cat. #71206-3]). Subsequently, the cells were resuspended in R0 media (RPMI+10% FBS, 1% Penn/Strep, 1% L-glutamine) and rested overnight. The rested cells were stimulated for 8 h with respective peptide pools (Table 38). Each peptide pool contained 1mer-peptides which were overlapping by 11 amino acids spanning the length of the relevant antigen. For stimulation, a concentration of 2 μg/ml of each peptide was used. In addition, cells were treated with GolgiStop and GolgiPlug as well as co-stimulated with anti-CD49d and anti-CD28 antibodies during stimulation. The stimulated cells were stained and fixed as outlined in Table 39 and analyzed on a BD Symphony flow cytometer.

TABLE 38
Re-Stimulation

| Re-stimulation Peptides | Sequence | Source | Lot # |
|---|---|---|---|
| Full SARS-COV-2 Spike | 315 15mers spanning Spike | GenScript | |
| Glycoprotein | Protein Sequence, | | |
| Peptide Pool Mix | overlap 11aa | | |
| Custom RBD (aa319-591) | 15mers spanning | GenScript | U240GGB120-235~240 |
| peptide pool | Spike RBD, | | |
| | overlap 11aa | | |
| | 54 peptides for backbone* | | |
| | 8 peptides for Beta | | |
| | 7 peptides for Delta | | |
| | 12 peptides for WT | | |

*backbone peptides are identical in all variants. For each stimulation concerning a specific variant form of RBD, a combination of the remaining peptides was added to the backbone peptides to complete the RBD sequence with the corresponding mutations.

TABLE 39
Antibodies used for ICS

| | | | | Catalog | Amount | | Time | |
|---|---|---|---|---|---|---|---|---|
| Specificity | Fluorochrome | Clone | Vendor | Number | (uL) | Step | [min] | Temp |
| Dead cells | Aqua | | Invitrogen | L34957 | 0.25 | Pre-Surface | 20 | RT |
| CD3 | BV786 | SP34.2 | BD | 557757 | 2.5 | Intracellular | 20 | RT |
| | | | Bioscience | | | | | |
| CD4 | PE-Cy5.5 | S3.5 | Invitrogen | MHCD0418 | 1 | Surface | 20 | RT |
| CD8 | Alexa 647 | RPA-T8 | BioLegend | 301022 | 0.25 | Surface | 20 | RT |
| CD69 | ECD | TP1.55.3 | Beckman | 6607110 | 10 | Intracellular | 20 | RT |
| | | | Coulter | | | | | |
| IFN-g | Ax700 | B27 | BioLegend | 506516 | 0.25 | Intracellular | 20 | RT |
| IL-2 | BV421 | MQ1- | BioLegend | 500328 | 5 ul | Intracellular | 20 | RT |
| | | 17H12 | | | | | | |
| IL-4 | PE | 8D4-8 | BioLegend | 500704 | 1 | Intracellular | 20 | RT |
| TNF-a | BV605 | Mab11 | BioLegend | 502936 | 5 | Intracellular | 20 | RT |
| IL-17A | PE-Cy7 | BL168 | BioLegend | 512315 | 0.5 | Intracellular | 20 | RT |
| CD45RA | BV711 | 5H9 | BD | 556626 | 5 | Surface | 20 | RT |
| | | | Bioscience | | | | | |
| CCR7 | BV650 | G043H7 | BioLegend | 353234 | 5 | Surface | 20 | RT |
| (CD197) | | | | | | | | |

RT = room temperature

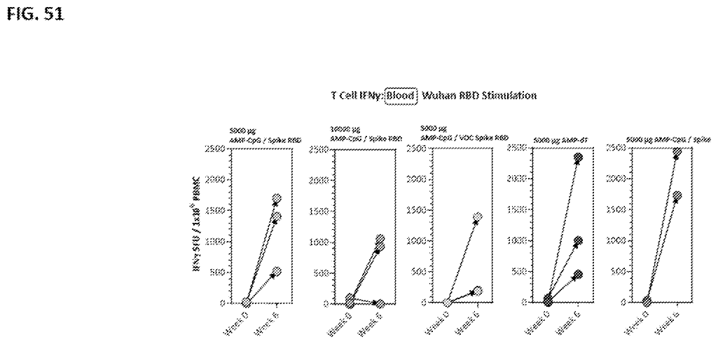

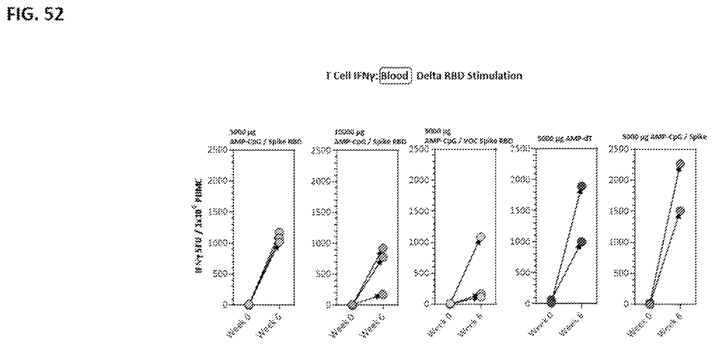

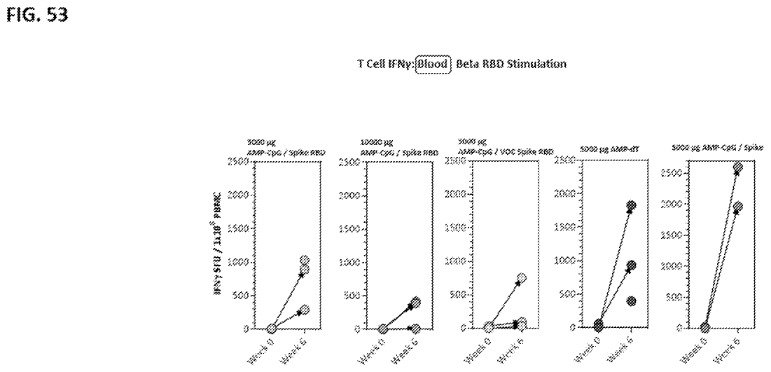

The PBMCs were handled and rested as described above. MabTech Monkey IFN-γ ELISpot PLUS kit HRP (3421M-4HPT-10) was used as described by the manufacturer. 0.1×10⁶ cells were used per well. The cells were activated with 0.1 μg/well (1 μg/ml) RBD peptide pool (Table 38). The IFNγ plates were stimulated overnight (20 h) and developed according to manufacturer's instructions. The results of these studies are shown in FIGS. 51-53.

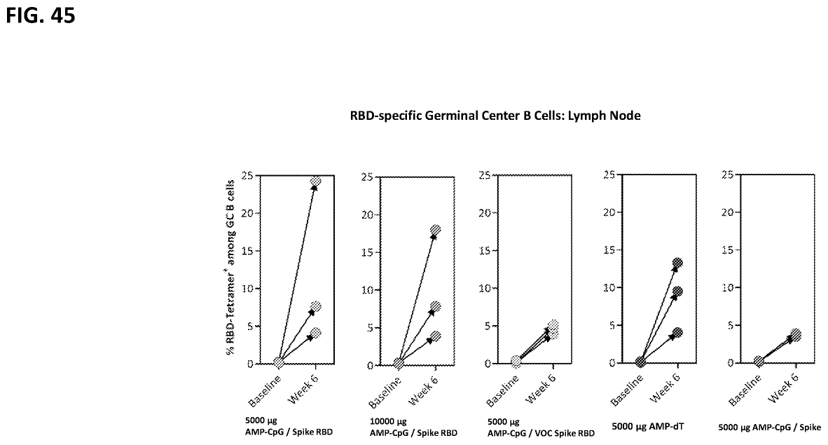

The tetramers prepared (fluorescently labelled RBD) were RBD-BV605, including a 1:2 ratio of biotinylated RBD+Streptavidin-BV605, and RBD-APC, including a 1:2 ratio of biotinylated RBD+Streptavidin-AP. The tetramers were made by incubating biotinylated-RBD with each SA-fluorochrome (separate reactions for each fluorochrome) at RT, for 20 minutes. The lymph node cells were stained in the following order: Fc Block and Live/Dead Stain, fluorescently labelled RBD, surface stain with subsequent fixation and permeabilization, and intracellular stain. Cells were analyzed on a BD Symphony flow cytometer. The antibodies used in this assay are described in Table 40. The results of the tetramer assay are shown in FIG. 45.

TABLE 40
Antibodies used for B-cell Tetramer

| | | | | Catalog | Amount | |
|---|---|---|---|---|---|---|
| Specificity | Fluorochrome | Clone | Vendor | Number | (per sample) | Step |
| IgM | FITC | G20-127 | BD | 555782 | 20 | Surface |
| IgG | PE-Cy7 | G18-145 | BD | 551497 | 5 | Surface |
| CD20 | PE-Dazzle 594 | 2H7 | BioLegend | 302348 | 5 | Surface |
| CD3 | AF700 | SP34-2 | BD | 557917 | 5 | Surface |
| CD4 | APC-Cy7 | OKT4 | BioLegend | 317418 | 5 | Surface |
| PD1 | BV650 | EH12 | BD | 564104 | 5 | Surface |
| CXCR5 | PerCp- | MUSUBEE | Thermo | 25-9185-42 | 5 | Surface |
| | Fluor710/PerCp5.5 | | | | | |
| Bcl6 | PE | 7D1 | BioLegend | 358503 | 5 | intranuclear |
| Ki67 | BV421 | 1156 | BioLegend | 151208 | 5 | intranuclear |
| RBD | Sav-BV605 | na | BioLegend | 405229 | refer protocol | tetramer |
| RBD | Sav-APC | na | BioLegend | 405207 | refer protocol | tetramer |
| Aqua | AmCyan | na | Invitrogen | L34957 | 1 | surface |

A pseudovirus neutralizing assay was performed by sending the serum samples to GenScript for analysis. The results of this study is shown in FIG. 44B.

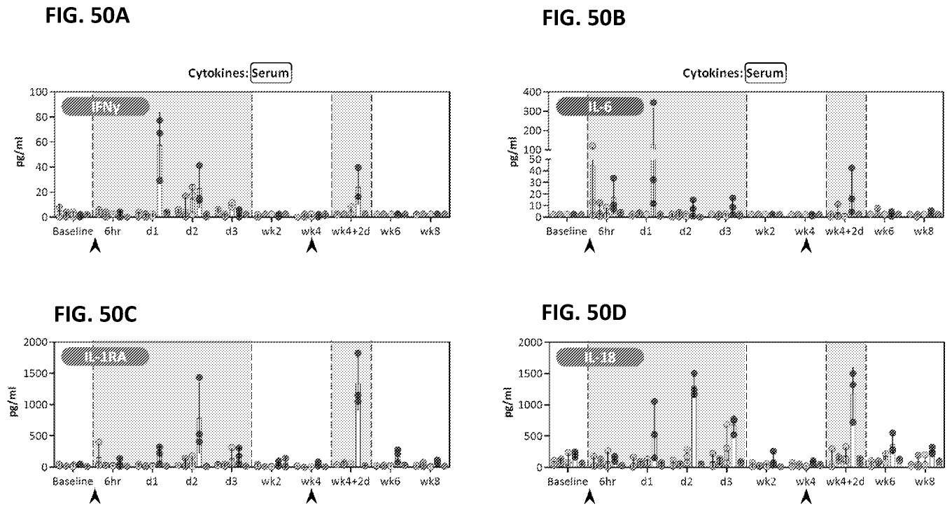

A serum cytokine luminex assay was performed by heat inactivating the serum. The Luminex assay was performed as indicated by the manufacturer using the kit used PCYTMG-40K-PX23. The amount of IFNγ (FIG. 50A), IL-6 (FIG. 50B), IL-i RA (FIG. 50C), and IL-18 (FIG. 50D) was measured for each sample.

No site reactogenicity (redness, swelling, itching) was observed. There were no changes in temperature (measured daily for one week post each dose). There were no major alterations in body weight (measured biweekly). There were no major changes in % neutrophils, lymphocytes, monocytes, eosinophils or basophils, platelets, RBC for 2 days post each dose. There were no major changes in chemistry panel (glucose, BUN, creatine, Na, K, Chloride, Ca, albumin, ALT, LDH, etc) for 2 days post each dose.

The AMP-vaccines were very well tolerated by the NHPs. There was no sign of toxicity even at the higher concentrations of AMP-CpG or AMP-dT. The antibody responses were strong after one dose, and further increased after the second dose. The animals that received 2 doses of AMP-vaccine showed responses greater than those reported in the literature for other CoV-2 vaccines that were tested on NHPs. The animals vaccinated with 3000 μg of AMP-CpG7909 and WT Spike RBD showed higher neutralizing antibody titers than those measured in convalescent patients. Furthermore, a robust B-cell populations and circulating T-cells specific to CoV2 Spike RBD were detected in lymph nodes and blood, respectively, of immunized NHPs.

### Example 9. Evaluating the Effect of Nucleotide Sequence in DNA Sensing

This experiment was designed to determine if nucleotide sequence is important in DNA sensing. Groups of 5 C57BL/6J were administered a vaccine including the components described in Table 41 and show in FIG. 54.

TABLE 41
Summary of Vaccine Administration in Mice

| | | | | Dosing and Sample Collection | | | |
|---|---|---|---|---|---|---|---|
| | | Vaccine Components | | Day 0 | Day 13 | Day 14 | Day 21 |
| Group | Treatment Name | Antigen (5 ug) | Adjuvant (5 nmol) | Dose 1 | Read-out | Dose 2 | Read-out |
| 1 | dA/dT-50-PS | GenScript RBD | 5′-sdT50-3′ (PS) | x | ICS | x | ICS on |
| | | | 5′-sdA50-3′ (PS) | | | | Lung and |
| 2 | AMP-dA/dT-50-PS | GenScript RBD | 5′-AMP-dT50-3′ (PS) | x | | x | PBMCs |
| | | | 5′-dA50-3′ (PS) | | | | Spleen |
| 3 | dAdT/dAdT-50-PS | GenScript RBD | 5′-dAdT50-3′ (PS) | x | | x | ELISpot |
| | | | 5′-dTdA50-3′ (PS) | | | | |
| 4 | AMP-dAdT/ | GenScript RBD | 5′-AMP-dAdT50-3′ (PS) | x | | x | |
| | dAdT-50-PS | | 5′-dTdA50-3′ (PS) | | | | |
| 5 | ISD-PS | GenScript RBD | 5′-ISD-3′ (PS) | x | | x | |
| | | | comp strand (PS) | | | | |
| 6 | AMP-ISD-PS | GenScript RBD | 5′-AMP-ISD-3′ (PS) | x | | x | |
| | | | comp strand (PS) | | | | |
| 7 | Mock Tx | GenScript RBD | — | x | | x | |

Adjuvant stock solutions were prepared by resuspending in limulus amebocyte lysate (LAL) H₂O. Final injections were diluted with 1×Phosphate-buffered saline (PBS) such that the AMP-dAdT and AMP-ISD nucleic acid sequence had a concentration of 5 nmol/100 μL injection. The SARS-CoV2 Spike S1 RBD protein stock solutions were prepared by dissolving the protein in PBS at a concentration of 0.88 mg/ml, and final injections were diluted with 1×PBS to a concentration of 5 μg/100 μL injection. The vaccine components are described in Table 42.

The immunizations were administered subcutaneously (SC) into the tail base of female B6 mice, bilaterally with 50 μL per side. A booster dose was given at roughly 2-week intervals. SC injections ensured that the vaccine was optimally delivered into lymph nodes via natural lymph drainage. Bi-weekly injections were determined to be optimal in immune response generation in previous mouse studies.

TABLE 42
Vaccine Components

| Vaccine Components | Sequence or Cat# | Source | Lot # |
|---|---|---|---|
| SARS-COV2 RBD, His | Z03483 | GenScript | B200931 |
| dA/dT-50-PS | 5′-sdT-3′ | AXO | XD-15740 K2 |
| | 5′-sdA-3′ | | |
| AMP-dA/dT-50-PS | 5′-AMP-sdT-3′ | AXO | XD-15739 K3 |
| | 5′-sdA-3′ | | |
| dAdT/dAdT-50-PS | 5′-sdAsdT-3′ | AXO | XD-28895 K1 |
| | 5′-sdTsdA-3′ | | |
| AMP-dAdT/dAdT-50-PS | 5′-AMP-sdAsdT-3′ | AXO | XD-28894 K1 |
| | 5′-sdTsdA-3′ | | |
| ISD-PS | ISD PS | AXO | XD-29671 K1 |
| | Comp strand PS | | |
| AMP-ISD-PS | AMP-ISD PS | AXO | XD-29672 K1 |
| | Comp strand PS | | |

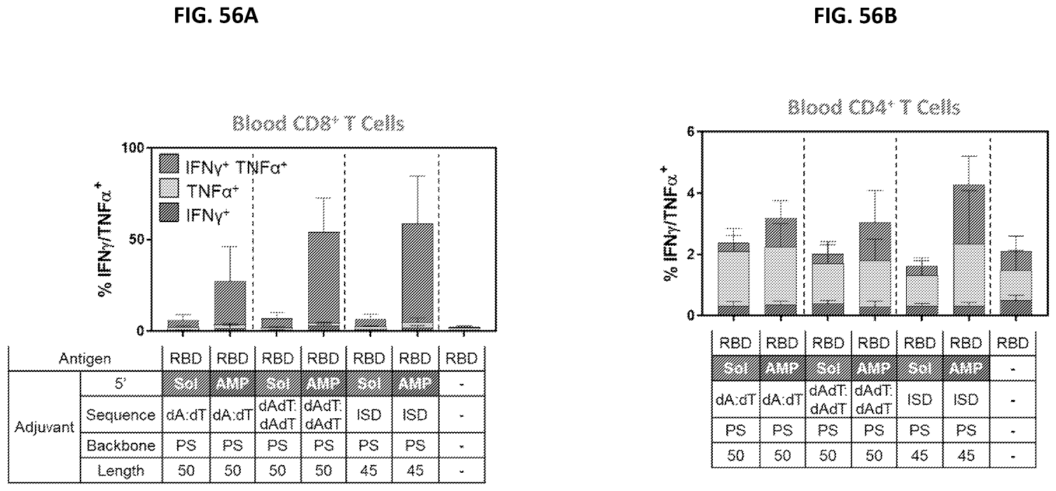

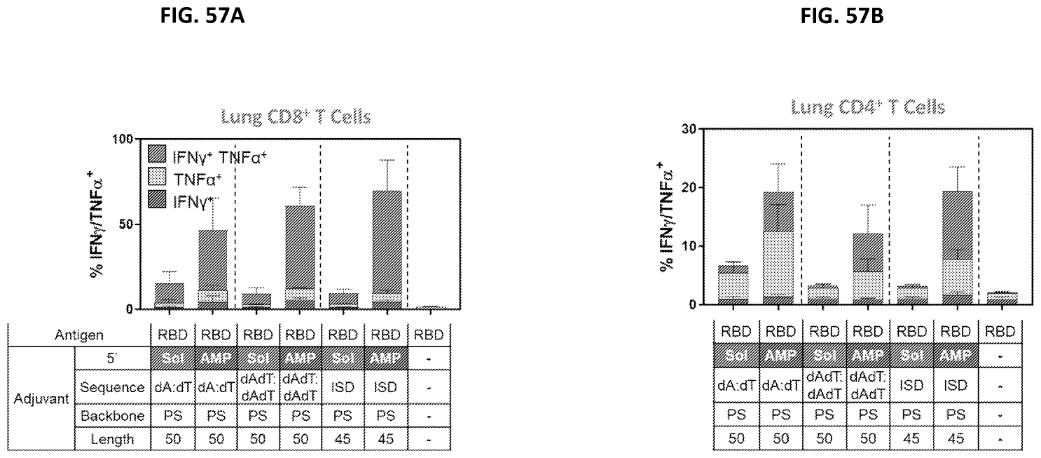

An ICS (Intracellular Stain) assay for TNFα and IFNγ was performed on PBMCs 7 days after dosing. An ICS was also performed on lung samples 7 days post dose 2 (FIGS. 56A, 56B, 57A, and 57B). The cells were also surface stained for CD4, CD8 and CD3 using the antibodies described in Table 43. ICS samples were activated overnight (in the presence of Brefeldin A and Monensin) with 1 μg/well of SARS-CoV-2 Spike Glycoprotein Peptide Pool Mix [315 peptides each at 1 μg/well](Table 44).

TABLE 43
Antibodies used for ICS

| Antigen | Color | Source | Product # | Lot # |
|---|---|---|---|---|
| TNFα | FITC | BD | 554418 | 9123915 |
| IFNγ | PE | BD | 554412 | 9154769 |
| CD8a | APC | eBioscience | 17-0081-83 | 4321418 |
| CD4 | PE-Cy7 | Invitrogen | 25-0041-82 | 2123767 |
| CD3 | APC-Cy7 | BD | 560590 | 9179637 |
| LiveDead | Aqua | Invitrogen | L34966 | 1832692 |
| Brefeldin A | — | Invitrogen | 00-4506-51 | 1915300 |
| Monensin | — | BioLegend | 420701 | B297750 |

TABLE 44
Re-Stimulation Peptides

| Re-stimulation | | | |
|---|---|---|---|
| Peptides | Sequence | Source | Lot # |
| SARS-COV-2 Spike | 315 15mers | GenScript | E5868620K |
| Glycoprotein | spanning Spike | | |
| Peptide Pool Mix | Protein Sequence, | | |
| | overlap 11aa | | |

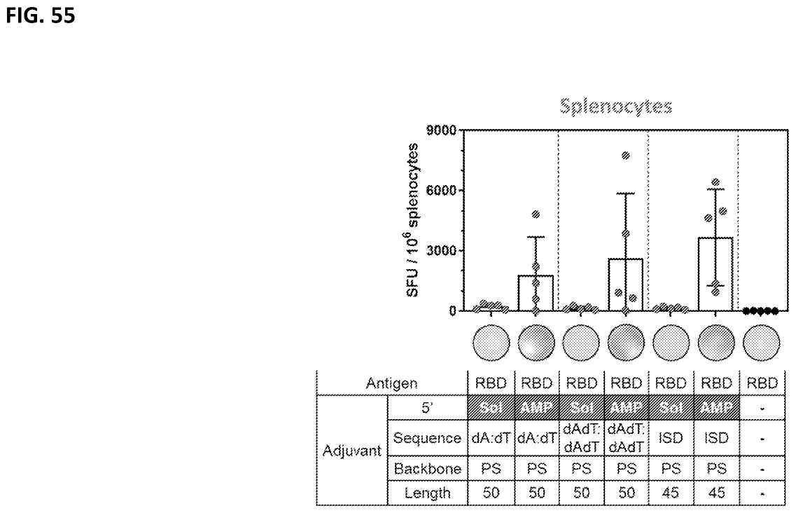

ELISpot analysis for IFNγ was performed on splenocytes after dose 2 administration (FIG. 55). Splenocytes (0.1×10⁶ cells/well) were activated with 1 μg/ml SARS-CoV-2 Spike Glycoprotein Peptide Pool Mix (Table 44). The IFNγ plates were stimulated overnight.

The immunogenicity of all three DNA sequences was increased significantly when conjugated to an amphiphile. Additionally, the number of CD8⁺ T cells was significantly increased in both circulation and lung, whereas CD4 T cells primarily appeared to accumulate in peripheral tissues such as the lung.

### Example 10. Evaluating the Change in the Transcriptome of Lymph Node Upon Vaccination

This experiment was designed to analyze the changes in the transcriptome of lymph node cells in the early stages upon vaccination with amphiphilic-adjuvants.

7 groups of 8 C57BL/6J were administered a vaccine including the components described in Table 45.

TABLE 45
Summary of Vaccine Administration in Mice

| | | | | | Assays | | | | |
|---|---|---|---|---|---|---|---|---|---|
| | Treatment | # of | Vaccine Components | | 0 h | 2 h | 6 h | 24 h | 72 h |
| Group | Name | Mice | Antigen (5 ug) | Adjuvant (5 nmol) | Dose 1 | Read-out | Read-out | Read-out | Read-out |
| 1 | AMP-dAdT | 2 | GenScript RBD | 5′-AMP-dT50-3′ | x | LN RNA | LN RNA | LN RNA | LN RNA |
| | | | | 5′-dA50-3′ | | | | | |
| 2 | Sol dAdT | 2 | GenScript RBD | 5′-dT50-3′ | x | | | | |
| | | | | 5′-dA50-3′ | | | | | |
| 3 | AMP-dT | 2 | GenScript RBD | 5′-AMP-dT50-3′ | x | | | | |
| 4 | Sol dT | 2 | GenScript RBD | 5′-dT50-3′ | x | | | | |
| 5 | AMP-CpG | 2 | GenScript RBD | AMP-CpG7909 (1 nmol) | x | | | | |
| 6 | Sol CpG | 2 | GenScript RBD | CpG7909 (1 nmol) | x | | | | |
| 7 | Mock | 4 | GenScript RBD | — | x | | | | |

Adjuvant stock solutions were prepared by resuspending in limulus amebocyte lysate (LAL) H20, and final injections were diluted with 1×Phosphate-buffered saline (PBS), such that the AMP-dAdT and AMP-dT had a final concentration of 5 nmol/100 μL injection and the amphiphilic AMP-CpG had a final concentration of 1 nmol/100 μL injection. The SARS-CoV2 Spike S1 RBD protein stock solutions were prepared by dissolving protein in PBS at a concentration of 0.89 mg/ml, and final injections were diluted with 1×PBS to a concentration of 5 μg/100 μL injection. The vaccine components are described in Table 46.

The immunizations were administered subcutaneously (SC) into the tail base of female B6 mice, bilaterally with 50 μL per side. A booster dose was given at roughly 2-week intervals. SC injections ensured that the vaccine was optimally delivered into lymph nodes via natural lymph drainage. Bi-weekly injections were determined to be optimal in immune response generation in previous mouse studies.

TABLE 46
Vaccine Components

| Vaccine | Sequence | | |
|---|---|---|---|
| Components | or Cat# | Source | Lot # |
| SARS-CoV2 | Z03483 | GenScript | B2009021 |
| RBD, His | | | |
| AMP-dT50 | 5′-AMP- | AXO | X-51175 K1 |
| | sdT₅₀-3′ | | |
| AMP-dAdT50 | 5′-AMP- | AXO | XD-15739 K4 |
| | dT50-3′ | | |
| | 5′-dA50-3′ | | |
| AMP- | 5′-(Diacyl | Avecia | 18-025-A |
| CpG7909 | lipid) | | |
| | tcgtcg | | |
| | ttttgt | | |
| | cgtttt | | |
| | gtcgt | | |
| | t-3′ | | |
| | (SEQ ID | | |
| | NO: 26) | | |

For the lymph node extraction, mice were sacrificed at the corresponding time points, and lymph nodes were extracted in media-containing microcentrifuge tubes. The lymph nodes were transferred onto a wet (1 ml of growth medium (RPMI) was added to the strainer to wet the surface) 70 μm nylon strainer and ground through the filter into a 50 mL tube using the plunger of a syringe. The filter was washed with 10 mL of RPMI.

The cell suspensions were then transferred into a 15 mL tube and spun for 5 mins 1750 rotations per minute (RPM) at a temperature of 4° C. The supernatant was aspirated carefully, and cell pellet was resuspended in 1 mL of RPMI. The cells were then counted and spun down again. The supernatant was aspirated thoroughly and Qiagen RLT buffer (stored at RT, kept sterile) was added to achieve a concentration of 2×10⁶ cells/mL. The cells were resuspended by pipetting up and down initially and then vortexed for at least 10 seconds. 0.2×10⁶ cells (100 μl) were shipped to NanoString. The rest was aliquoted and stored at −80° C.

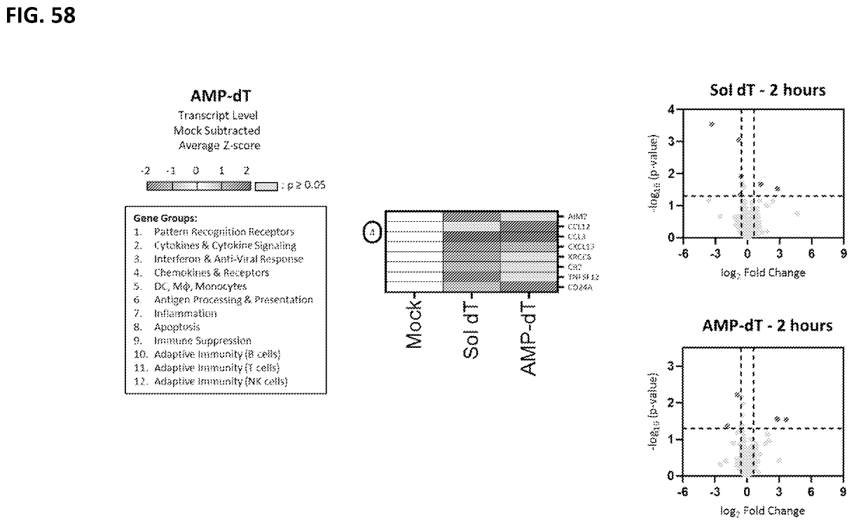

The results showed that for AMP-dT, chemokines were upregulated as early as 2 h post immunization (FIG. 58), which could indicate the induction of a potent inflammatory environment in the lymph node that results in the recruitment of additional leukocytes.

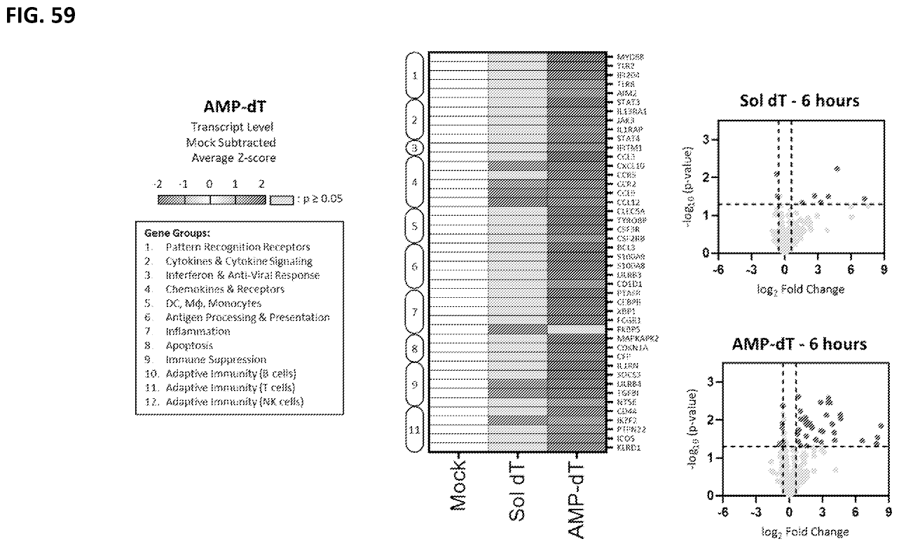

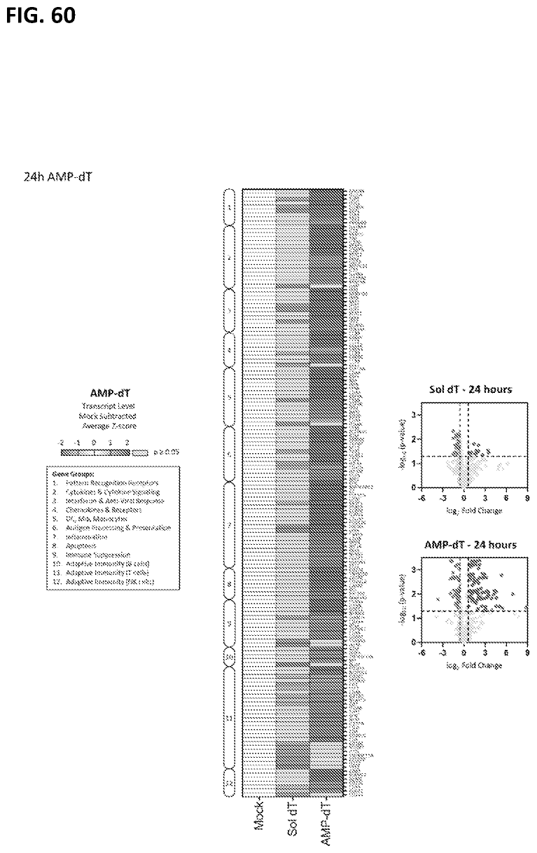

From 6 to 24 h many more immunologically relevant genes were upregulated. Among these are pattern recognition receptors, antigen processing and presentation genes, and antigen presenting cell (APC) activation markers that indicate that the innate immune system is being activated. Concomitantly, chemokines, cytokines, interferons, anti-viral and inflammatory proteins are highly upregulated, which could be a consequence of the innate immune system activation (FIGS. 59 and 60). On the contrary, genes that are associated with the adaptive immune system are, as expected, mostly downregulated at this early point in time.

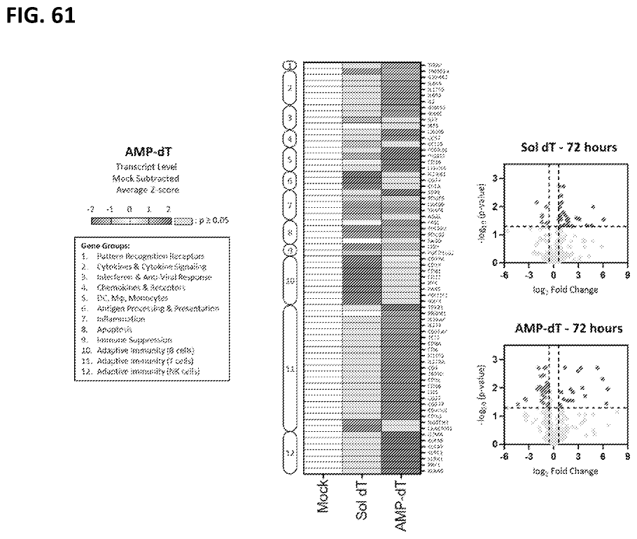

At 72 h post immunization it appeared that the initial inflammatory response was subsiding (FIG. 61). APC markers were still elevated. An increase in the expression of genes associated with NK cells may suggest an influx of those cells into the lymph node at that time.

For soluble dT, on the other hand, not many genes were upregulated in the first 24 h (FIGS. 58, 59, and 60). At 72 h, a gene signature that may suggest a predominantly B cell rich environment can be observed (FIG. 61).

### Example 11. Evaluating a Nucleic Acid Sequence Derived from Herpes Simplex Virus as an Adjuvant

This experiment was designed to determine if the AMP-DNA adjuvant Herpes Simplex Virus (HSV-60), which consists of a 60 nucleic acid residue sequence from the Herpes Simplex Virus, is a good adjuvant and whether the sequence is required to be in its double stranded conformation or if the sense-strand is sufficient to elicit an immune response.

5 groups of 5 C57BL/6J were administered a vaccine including the components described in Table 47 and shown in FIG. 62.

TABLE 47
Summary of Vaccine Administration in Mice

| | | | | Dosing and Sample Collection | | |
|---|---|---|---|---|---|---|
| | Treatment | Vaccine Components | | Day 0 | Day 14 | Day 21 |
| Group | Name | Antigen (5 ug) | Adjuvant (5 nmol) | Dose 1 | Dose 2 | Read-out |
| 1 | DS AMP-HSV60 | GenScript RBD | AMP-5′-HSV-3′ Sense | x | x | ICS on |
| | | | 5′-HSV-3′ Antisense | | | |
| 2 | DS HSV60 | GenScript RBD | 5′-HSV-3′ Sense | x | x | Lung and |
| | | | 5′-HSV-3′ Antisense | | | |
| 3 | SS AMP-HSV60 | GenScript RBD | AMP-5′-HSV-3′ Sense | x | x | PBMCs |
| 4 | SS HSV60 | GenScript RBD | 5′-HSV-3′ Sense | x | x | Spleen |
| | | | | | | ELISpot |
| 5 | Mock Tx | GenScript RBD | — | x | x | |

The HSV-60 sequence sense strand has the following sequence (5′ to 3′) TAAGACACGATGCGATAAAATCTGTTTGTAAAATTTATTAAGGGTACAAATTGCCCTAGC (SEQ ID NO: 34). The HSV-60 sequence anti-sense strand has the following sequence (5′ to 3′) GCTAGGGCAATTTGTACCCTTAATAAATTTTACAAACAGATTTTATCGCATCGTGTCTTA (SEQ ID NO: 35) as is shown in FIG. 62, where the 5′ end of the sense strand is bonded to the diacyl lipid.

Adjuvant stock solutions were prepared by resuspending in limulus amebocyte lysate (LAL) H₂O, and final injections were diluted with 1×Phosphate-buffered saline (PBS) such that the concentration of AMP-HSV60 was 5 nmol/100 μL injection. The SARS-CoV2 Spike S1 RBD protein stock solutions were prepared by dissolving the protein in PBS at a concentration of 1.14 mg/mL, and final injections were diluted with 1×PBS to a final concentration of 5 μg/100 μL injection. The vaccine components are described in Table 48.

The immunizations were administered subcutaneously (SC) into the tail base of female B6 mice, bilaterally with 50 μL per side. A booster dose was given at roughly 2-week intervals. SC injections ensured that the vaccine was optimally delivered into lymph nodes via natural lymph drainage. Bi-weekly injections were determined to be optimal in immune response generation in previous mouse studies.

TABLE 48
Vaccine Components

| Vaccine Components | Sequence or Cat# | Source | Lot # |
|---|---|---|---|
| SARS-COV2 RBD, His | Z03483 | GenScript | B2009033 |
| DS AMP-HSV60 | AMP-5′-HSV-3′ Sense | AXO | XD-35681 |
| | 5′-HSV-3′ Antisense | | |
| DS HSV60 | 5′-HSV-3′ Sense | AXO | XD-35682 |
| | 5′-HSV-3′ Antisense | | |
| SS AMP-HSV60 | AMP-5′-HSV-3′ Sense | AXO | X-102090 |
| SS HSV60 | 5′-HSV-3′ Sense | AXO | X-102091 |

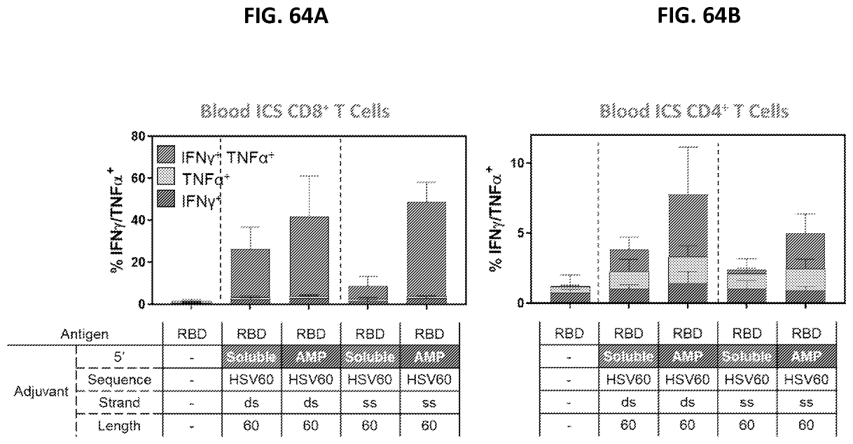

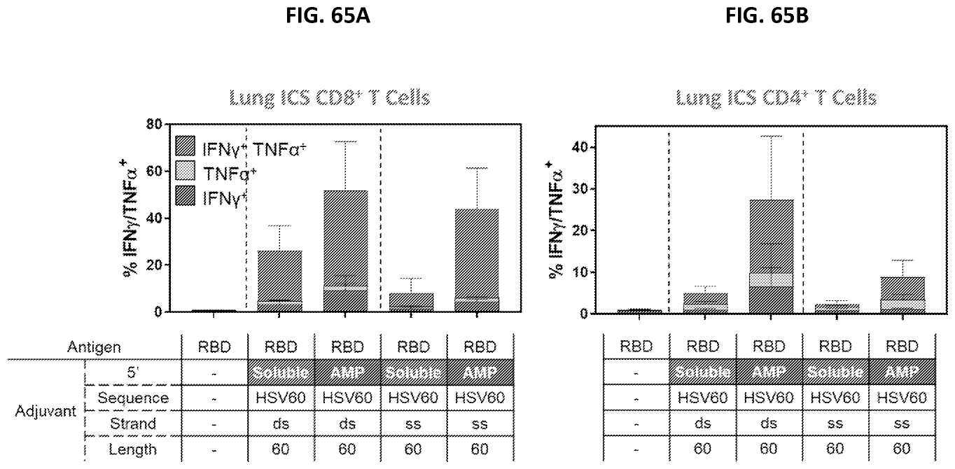

An ICS (Intracellular Stain) assay for TNFα and IFNγ was performed on PBMCs 7 days after dosing. An ICS was also performed on lung samples 7 days post dose 2 (FIGS. 64A, 64B, 65A, and 65B). The cells were also surface stained for CD4, CD8 and CD3 using the antibodies described in Table 49. ICS samples were activated overnight (in the presence of Brefeldin A and Monensin) with 1 μg/well of SARS-CoV-2 Spike Glycoprotein Peptide Pool Mix [315 peptides each at 1 μg/well](Table 50).

TABLE 49
Antibodies used for ICS

| Antigen | Color | Source | Product # | Lot # |
|---|---|---|---|---|
| TNFα | FITC | BD | 554418 | 9123915 |
| IFNγ | PE | BD | 554412 | 9154769 |
| CD8a | APC | eBioscience | 17-0081-83 | 4321418 |
| CD4 | PE-Cy7 | Invitrogen | 25-0041-82 | 2123767 |
| CD3 | APC-Cy7 | BD | 560590 | 9179637 |
| LiveDead | Aqua | Invitrogen | L34966 | 1832692 |
| Brefeldin A | — | Invitrogen | 00-4506-51 | 1915300 |
| Monensin | — | BioLegend | 420701 | B297750 |

TABLE 50
Re-Stimulation Peptides

| Re-stimulation | | | |
|---|---|---|---|
| Peptides | Sequence | Source | Lot # |
| SARS-COV-2 Spike | 315 15mers | GenScript | |
| Glycoprotein | spanning Spike | | |
| Peptide Pool Mix | Protein Sequence, | | |
| | overlap 11aa | | |

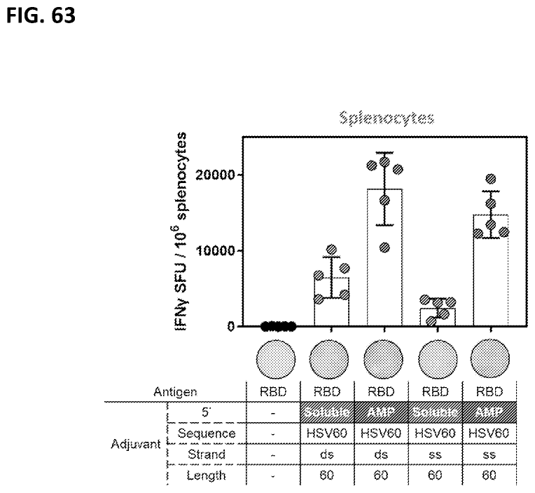

ELISpot analysis for IFNγ was performed on splenocytes after dose 2 administration (FIG. 63). Splenocytes (0.1×10⁶ cells/well) were activated with 1 μg/well PepMix (Table 50). The IFNγ plates were stimulated overnight.

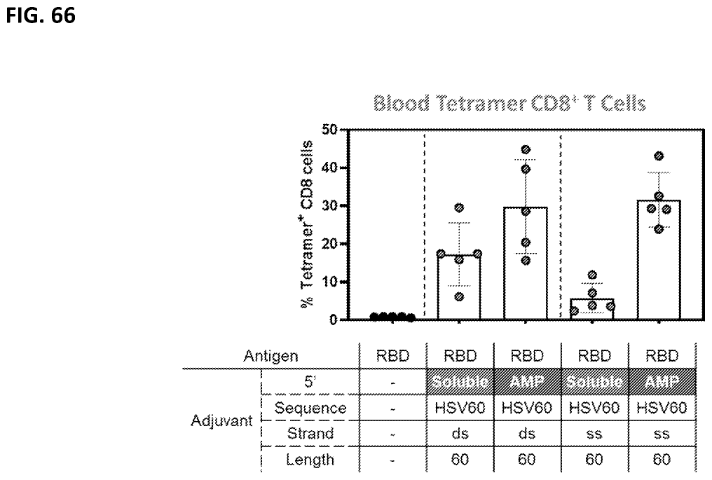

Tetramer analysis was performed 7 days post dosing using the H-2Kb CoV2 RBD Tetramer-VNFNFNGL-PE (SEQ ID NO: 25) (NIH 53309) (FIG. 66).

The AMP-modification of the HSV-60 adjuvant increased immunogenicity. Both single-stranded and double-stranded HSV-60 benefited from the AMP-conjugation. There was not much difference in the immune response elicited by the single-stranded and double-stranded variants of HSV-60 adjuvant. However, the double-stranded variant tended to perform better. This may have been due to the equimolar quantities used, which translates into twice as much DNA being present in the double-stranded variant.

### Example 12. Evaluating if Both Strands of ISD are Necessary of Immune Response

This experiment was designed to determine if both strands of the ISD sequence were necessary to elicit a strong immune response, or, if only one was needed, which one is more immunogenic.

7 groups of 5 C57BL/6J were administered a vaccine including the components described in Table 51 and shown in FIG. 67.

TABLE 51
Summary of Vaccine Administration in Mice

| | | | | Dosing and Sample Collection | | | |
|---|---|---|---|---|---|---|---|
| | | Vaccine Components | | Day 0 | Day 13 | Day 14 | Day 21 |
| Group | Treatment Name | Antigen (5 ug) | Adjuvant (5 nmol) | Dose 1 | Read-out | Dose 2 | Read-out |
| 1 | ISD | GenScript RBD | 5′-ISD-3′ Sense | x | ICS | x | ICS on |
| | | | 3′-ISD-5′ Anti-Sense | | | | Lung and |
| 2 | AMP-ISD | GenScript RBD | 5′-AMP-ISD-3′ Sense | x | | x | PBMCs |
| | | | 3′-ISD-5′ Anti-Sense | | | | Spleen |
| 3 | Sense | GenScript RBD | 5′-ISD-3′ Sense | x | | x | ELISpot |
| 4 | AMP-Sense | GenScript RBD | 5′-AMP-ISD-3′ Sense | x | | x | |
| 5 | Antisense | GenScript RBD | 3′-ISD-5′ Anti-Sense | x | | x | |
| 6 | AMP-Antisense | GenScript RBD | 3′-AMP-ISD-5′ Anti- | x | | x | |
| | | | Sense | | | | |
| 9 | Mock Tx | GenScript RBD | — | x | | x | |

Adjuvant stock solutions were prepared by resuspending in limulus amebocyte lysate (LAL) H₂O, and final injections were diluted with 1×Phosphate-buffered saline (PBS) such that AMP-ISD was at a concentration of 5 nmol/100 μL injection and AMP-RNA was at a concentration of 5 nmol/100 μL injection (which equals 33 μg of RNA). SARS-CoV2 Spike S1 RBD protein stock solutions were dissolved in PBS at a concentration of 0.89 mg/ml, and final injections were diluted with 1×PBS with a concentration of 5 μg/100 μL injection. The vaccine components are described in Table 52.

The immunizations were administered subcutaneously (SC) into the tail base of female B6 mice, bilaterally with 50 μL per side. A booster dose was given at roughly 2-week intervals. SC injections ensured that the vaccine was optimally delivered into lymph nodes via natural lymph drainage. Bi-weekly injections were determined to be optimal in immune response generation in previous mouse studies.

TABLE 52
Vaccine Components

| Vaccine | | | |
|---|---|---|---|
| Components | Sequence or Cat# | Source | Lot # |
| SARS-COV2 | Z03483 | GenScript | B2009021 |
| RBD, His | | | |
| ISD | 5′-ISD-3′ Sense | AXO | XD-29671 K1 |
| | 3′-ISD-5′ Anti-Sense | | |
| AMP-ISD | 5′-AMP-ISD-3′ Sense | AXO | XD-29672 K1 |
| | 3′-ISD-5′ Anti-Sense | | |
| Sense | 5′-ISD-3′ Sense | AXO | X-88298 K1-V2 |
| AMP-Sense | 5′-AMP-ISD-3′ Sense | AXO | X-88300 K2 |
| Antisense | 3′-ISD-5′ Anti-Sense | AXO | X-88299 K1-V2 |
| AMP-Antisense | 3'-AMP-ISD-5′ Anti-Sense | AXO | X-100357 K1 |

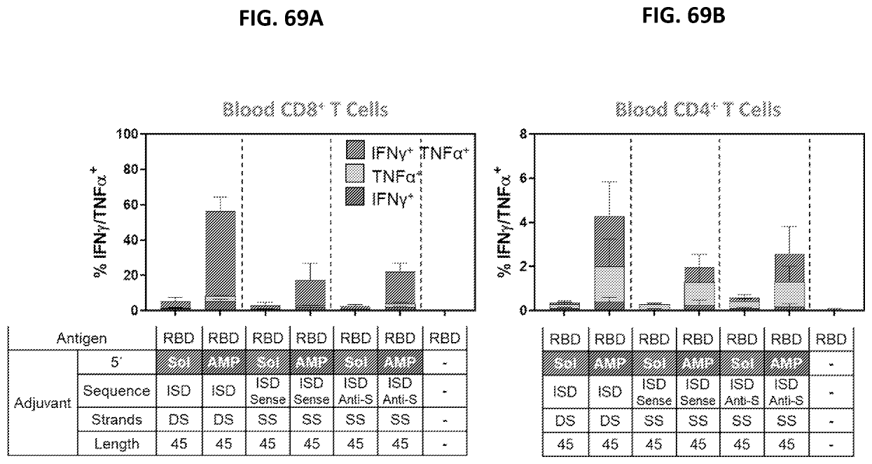

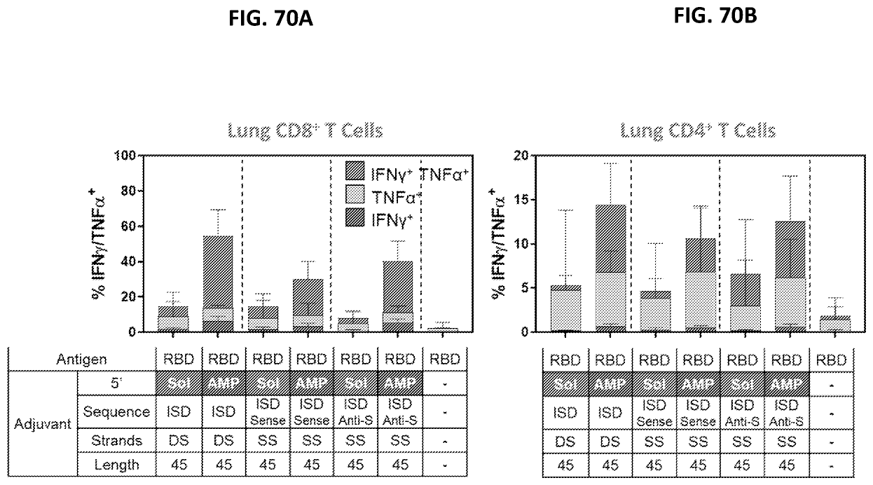

An ICS (Intracellular Stain) assay for TNFα and IFNγ was performed on PBMCs 7 days after dosing. An ICS was also performed on lung samples 7 days post dose 2 (FIGS. 69A, 691B, 70A, and 70B). The cells were also surface stained for CD4, CD8 and CD3 using the antibodies described in Table 53. ICS samples were activated overnight (in the presence of Brefeldin A and Monensin) with 1 μg/well of SARS-CoV-2 Spike Glycoprotein Peptide Pool Mix [315 peptides each at 1 μg/well](Table 54).

TABLE 53
Antibodies used for ICS

| Antigen | Color | Source | Product # | Lot # |
|---|---|---|---|---|
| TNFα | FITC | BD | 554418 | 9123915 |
| IFNγ | PE | BD | 554412 | 9154769 |
| CD8a | APC | eBioscience | 17-0081-83 | 4321418 |
| CD4 | PE-Cy7 | Invitrogen | 25-0041-82 | 2123767 |
| CD3 | APC-Cy7 | BD | 560590 | 9179637 |
| LiveDead | Aqua | Invitrogen | L34966 | 1832692 |
| Brefeldin A | — | Invitrogen | 00-4506-51 | 1915300 |
| Monensin | — | BioLegend | 420701 | B297750 |

TABLE 54
Re-Stimulation Peptides

| Re-stimulation | | | |
|---|---|---|---|
| Peptides | Sequence | Source | Lot # |
| SARS-COV-2 Spike | 315 15mers | GenScript | E5868620K |
| Glycoprotein | spanning Spike | | |
| Peptide Pool Mix | Protein Sequence, | | |
| | overlap 11aa | | |

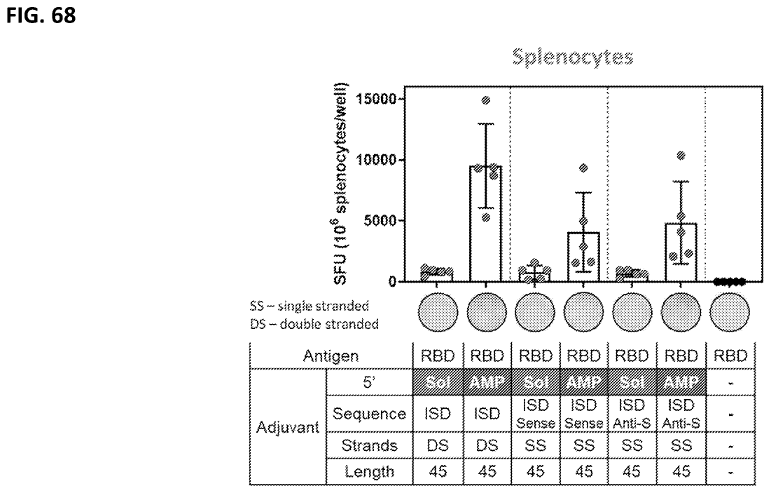

ELISpot analysis for IFNγ was performed on splenocytes after dose 2 administration (FIG. 68). Splenocytes (0.1×10⁶ cells/well) were activated with 1 μg/well PepMix (Table 54). The IFNγ plates were stimulated overnight.

The results of the experiment indicate that AMP-conjugation of any of the ISD variants improved their immunogenicity. The immune response for each of the single stranded variants was about half as strong as that of the double stranded ISD variant. This was most likely due to the fact that molar equivalents were used for each variant, and the double stranded version had twice the mass of the single stranded versions. Among the single stranded versions that was no significant difference in the immune response that was elicited.

### NUMBERED EMBODIMENTS

1. A compound comprising a poly-deoxyadenosine (poly-dA) nucleic acid sequence and an albumin-binding domain, or a pharmaceutically acceptable salt thereof.
2. A compound comprising a poly-deoxythymidine (poly-dT) nucleic acid sequence and an albumin binding moiety, or a pharmaceutically acceptable salt thereof.
3. The compound or pharmaceutically acceptable salt thereof of embodiment 1 or embodiment 2, wherein the compound or pharmaceutically acceptable salt thereof comprises a poly-dA nucleic acid sequence and a poly-dT nucleic acid sequence.
4. The compound or pharmaceutically acceptable salt thereof of embodiment 3, wherein the poly-dA nucleic acid sequence and the poly-dT nucleic acid sequence hybridize to form a double-stranded DNA sequence.
5. The compound or pharmaceutically acceptable salt thereof of any one of embodiments 1-4, wherein the poly-dA nucleic acid sequence and/or poly-dT nucleic acid sequence comprise between 30 and 100 nucleotides.
6. The compound or pharmaceutically acceptable salt thereof of embodiment 5, wherein the poly-dA nucleic acid sequence and/or poly-dT nucleic acid sequence comprise between 50 and 100 nucleotides.
7. The compound or pharmaceutically acceptable salt thereof of embodiment 5, wherein the poly-dA nucleic acid sequence and/or poly-dT nucleic acid sequence comprise between 30 and 50 nucleotides.
8. The compound or pharmaceutically acceptable salt thereof of embodiment 5, wherein the poly-dA nucleic acid sequence and/or poly-dT nucleic acid sequence comprise 30, 40, 50, 75, or 100 nucleotides.
9. The compound or pharmaceutically acceptable salt thereof of any one of embodiments 3-8, wherein the poly-dA nucleic acid sequence and poly-dT nucleic acid sequence comprise the same number of nucleotides.
10. The compound or pharmaceutically acceptable salt thereof of any one of embodiments 1-9, wherein the poly-dA nucleic acid sequence and/or poly-dT nucleic acid sequence comprises a mixture of dA and dT nucleic acid residues.
11. The compound or pharmaceutically acceptable salt thereof of embodiment 10, wherein the poly-dA nucleic acid sequence comprises between 100% and 51% dA nucleic acid residues and between 0% and 49% dT nucleic acid residues.
12. The compound or pharmaceutically acceptable salt thereof of embodiment 10, wherein the poly-dT nucleic acid sequence comprises between 100% and 51% dT nucleic acid residues and between 0% and 49% dA nucleic acid residues.
13. The compound or pharmaceutically acceptable salt thereof of any one of embodiments 1-12, wherein at least one internucleotide group connecting the nucleotides in the poly-dA nucleic acid sequence and poly-dT nucleic acid sequence is a phosphodiester.
14. The compound or pharmaceutically acceptable salt thereof of any one of embodiments 1-12, wherein all internucleotide groups connecting the nucleotides in the poly-dA nucleic acid sequence and poly-dT nucleic acid sequence are phosphorothioate.
15. The compound or pharmaceutically acceptable salt thereof of any one of embodiments 1-14, wherein the albumin-binding domain is bonded to the 5′ end of the poly-dA nucleic acid sequence.
16. The compound or pharmaceutically acceptable salt thereof of any one of the embodiments 1-14, wherein the albumin-binding domain is bonded to the 5′ end of the poly-dT nucleic acid sequence.
17. A compound comprising an interferon stimulatory DNA (ISD) sequence and an albumin-binding domain or an immunostimulatory herpes simplex virus (HSV) sequence and an albumin-binding domain, or a pharmaceutically acceptable salt thereof.
18. The compound or pharmaceutically acceptable salt thereof of embodiment 17, wherein the albumin-binding domain is bonded to the 5′ end of the ISD sequence or the immunostimulatory HSV sequence.
19. The compound or pharmaceutically acceptable salt thereof of any one of embodiments 1-18, wherein the albumin-binding is a lipid.
20. The compound or pharmaceutically acceptable salt thereof of embodiment 19, wherein the lipid is a diacyl lipid.
21. The compound or pharmaceutically acceptable salt thereof of embodiment 20, wherein the diacyl lipid comprises acyl chains comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29, or 30 hydrocarbon units.
22. The compound or pharmaceutically acceptable salt thereof of embodiment 20 or embodiment 21, wherein the lipid is 1,2-distearoyl-sn-glycero-3-phosphouthanoia mine (DSPE).
23. The compound or pharmaceutically acceptable salt thereof of any one of embodiments 20-22, wherein the poly-dA nucleic acid sequence, the poly-dT nucleic acid sequence, or the ISD sequence is bonded or linked by a linker to the following lipid:

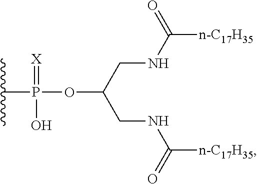

or a salt thereof,
wherein X is O or S.
24. The compound or pharmaceutically acceptable salt thereof of embodiment 23, wherein the linker is selected from the group consisting of a hydrophilic polymer, a string of hydrophilic amino acids, a polysaccharide, and an oligonucleotide, or a combination thereof.
25. The compound or pharmaceutically acceptable salt thereof of embodiment 23, wherein the linker comprises “N” polyethylene glycol units, wherein N is between 24-50.
26. The compound or pharmaceutically acceptable salt thereof of embodiment 25, wherein the linker comprises PEG24-amido-PEG24.
27. A poly-dA and poly-dT double-stranded DNA sequence comprising between 30 and 100 paired nucleotides.
28. The poly-dA and poly-dT double stranded DNA sequence of embodiment 27, wherein the poly-dA and poly-dT comprise the same number of nucleotides, or a pharmaceutically acceptable salt thereof.
29. A poly-dA or poly-dT single-stranded DNA sequence comprising between 30 and 100 nucleotides, or a pharmaceutically acceptable salt thereof.
30. A compound comprising a poly-deoxyguanosine (poly-dG) nucleic acid sequence nucleic acid sequence and an albumin-binding domain, or a pharmaceutically acceptable salt thereof.
31. A compound comprising a poly-deoxycytosine (poly-dC) nucleic acid sequence and an albumin binding moiety, or a pharmaceutically acceptable salt thereof.
32. The compound or pharmaceutically acceptable salt thereof of embodiment 30 or embodiment 31, wherein the compound or pharmaceutically acceptable salt thereof comprises a poly-dG nucleic acid sequence and a poly-dC nucleic acid sequence.
33. The compound or pharmaceutically acceptable salt thereof of embodiment 32, wherein the poly-dG nucleic acid sequence and the poly-dC nucleic acid sequence hybridize to form a double-stranded DNA sequence.
34. The compound or pharmaceutically acceptable salt thereof of any one of embodiments 30-33, wherein the poly-dG nucleic acid sequence and/or poly-dC nucleic acid sequence comprise between 30 and 100 nucleotides.
35. The compound or pharmaceutically acceptable salt thereof of embodiment 34, wherein the poly-dG nucleic acid sequence and/or poly-dC nucleic acid sequence comprise between 50 and 100 nucleotides.
36. The compound or pharmaceutically acceptable salt thereof of embodiment 34, wherein the poly-dG nucleic acid sequence and/or poly-dC nucleic acid sequence comprise between 30 and 50 nucleotides.
37. The compound or pharmaceutically acceptable salt thereof of embodiment 34, wherein the poly-dG nucleic acid sequence and/or poly-dC nucleic acid sequence comprise 30, 40, 50, 75, or 100 nucleotides.
38. The compound or pharmaceutically acceptable salt thereof of any one of embodiments 32-37, wherein the poly-dG nucleic acid sequence and poly-dC nucleic acid sequence comprise the same number of nucleotides.
39. The compound or pharmaceutically acceptable salt thereof of any one of embodiments 30-38, wherein the poly-dG nucleic acid sequence and/or poly-dC nucleic acid sequence comprises a mixture of dG and dC nucleic acid residues.
40. The compound or pharmaceutically acceptable salt thereof of embodiment 39, wherein the poly-dG nucleic acid sequence comprises between 100% and 51% dG nucleic acid residues and between 0% and 49% dC nucleic acid residues.
41. The compound or pharmaceutically acceptable salt thereof of embodiment 39, wherein the poly-dC nucleic acid sequence comprises between 100% and 51% dC nucleic acid residues and between 0% and 49% dG nucleic acid residues.
42. The compound or pharmaceutically acceptable salt thereof of any one of embodiments 30-41, wherein at least one internucleotide group connecting the nucleotides in the poly-dG nucleic acid sequence and poly-dC nucleic acid sequence is a phosphodiester.
43. The compound or pharmaceutically acceptable salt thereof of any one of embodiments 30-41, wherein all internucleotide groups connecting the nucleotides in the poly-dG nucleic acid sequence and poly-dC nucleic acid sequence are phosphorothioate.
44. The compound or pharmaceutically acceptable salt thereof of any one of embodiments 30-43, wherein the albumin-binding domain is bonded to the 5′ end of the poly-dG nucleic acid sequence.
45. The compound or pharmaceutically acceptable salt thereof of any one of the embodiments 30-43, wherein the albumin-binding domain is bonded to the 5′ end of the poly-dC nucleic acid sequence.
46. The compound or pharmaceutically acceptable salt thereof of any one of embodiments 30-45, wherein the albumin-binding is a lipid.
47. The compound or pharmaceutically acceptable salt thereof of embodiment 46, wherein the lipid is a diacyl lipid.
48. The compound or pharmaceutically acceptable salt thereof of embodiment 47, wherein the diacyl lipid comprises acyl chains comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29, or 30 hydrocarbon units.
49. The compound or pharmaceutically acceptable salt thereof of embodiment 47 or embodiment 48, wherein the lipid is 1,2-distearoyl-sn-glycero-3-phosphoethanolamine (DSPE).
50. The compound or pharmaceutically acceptable salt thereof of any one of embodiments 47-49, wherein the poly-dG nucleic acid sequence and/or the poly-dC nucleic acid sequence is bonded or linked by a linker to the following lipid:

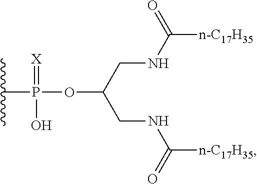

or a salt thereof,
wherein X is O or S.
51. The compound or pharmaceutically acceptable salt thereof of embodiment 50, wherein the linker is selected from the group consisting of a hydrophilic polymer, a string of hydrophilic amino acids, a polysaccharide, and an oligonucleotide, or a combination thereof.
52. The compound or pharmaceutically acceptable salt thereof of embodiment 51, wherein the linker comprises “N” polyethylene glycol units, wherein N is between 24-50.
53. The compound or pharmaceutically acceptable salt thereof of embodiment 52 wherein the linker comprises PEG24-amido-PEG24.
54. A poly-dG and poly-dC double-stranded DNA sequence comprising between 30 and 100 paired nucleotides.
55. The poly-dG and poly-dC double stranded DNA sequence of embodiment 54, wherein the poly-dG and poly-dC comprise the same number of nucleotides, or a pharmaceutically acceptable salt thereof.
56. A poly-dG or poly-dC single-stranded DNA sequence comprising between 30 and 100 nucleotides, or a pharmaceutically acceptable salt thereof.
57. A method of inducing an immune response against an antigen in a subject, the method comprising administering a compound, poly-dA and poly-dT double-stranded DNA sequence, poly-dA or poly-dT single-stranded DNA sequence, poly-dG and poly-dC double-stranded DNA sequence, poly-dG or poly-dC single-stranded DNA sequence, or pharmaceutically acceptable salt of thereof of any one of embodiments 1-56 and an antigen to the subject.
58. The method of embodiment 57, further comprising administering an adjuvant to the subject.
59. The method of embodiment 57 or embodiment 58, wherein the antigen is an influenza antigen, or fragment thereof.
60. The method of embodiment 59, wherein the antigen is an Influenza nucleoprotein, or fragment thereof.
61. The method of embodiment 60, wherein the Influenza nucleoprotein comprises a polypeptide sequence having at least 85% sequence identity to SEQ ID NO: 22.
62. The method of embodiment 61, wherein the Influenza nucleoprotein comprises a polypeptide sequence having at least 95% sequence identity to SEQ ID NO: 22.
63. The method of embodiment 62, wherein the Influenza nucleoprotein comprises a polypeptide sequence of SEQ ID NO: 22.
64. The method of embodiment 57 or embodiment 58, wherein the antigen is a coronavirus antigen, or fragment thereof.
65. The method embodiment 64, wherein the antigen is a coronavirus spike protein, or fragment thereof.
66. The method of embodiment 64, wherein the antigen is a coronavirus nucleocapsid protein, or fragment thereof.
67. The method of any of one of embodiments 57-66, wherein the compound or pharmaceutically salt thereof of any one of embodiments 1-56 is administered subcutaneously.
68. The method of any one of embodiments 57-67, the antigen is administered intramuscularly, subcutaneously, intravenously, intraperitoneally, topically, or orally.
69. The method of any one of embodiments 57-68, wherein the subject is a mammal.
70. The method of embodiment 67, wherein the subject is a human.
71. A pharmaceutical composition comprising a compound, poly-dA and poly-dT double-stranded DNA sequence, poly-dA or poly-dT single-stranded DNA sequence, poly-dG and poly-dC double-stranded DNA sequence, poly-dG or poly-dC single-stranded DNA sequence, or pharmaceutically acceptable salt thereof of any one of embodiments 1-56 and an antigen, or a nucleic acid sequence encoding the antigen, and a pharmaceutically acceptable carrier.
72. The pharmaceutical composition of embodiment 71, wherein the antigen is an influenza antigen or fragment thereof.
73. The pharmaceutical composition of embodiment 72, wherein the antigen is an influenza nucleoprotein or fragment thereof.
74. The pharmaceutical composition of embodiment 71, wherein the antigen is a coronavirus antigen, or fragment thereof.
75. A kit comprising a compound, poly-dA and poly-dT double-stranded DNA sequence, poly-dA or poly-dT single-stranded DNA sequence, poly-dG and poly-dC double-stranded DNA sequence, poly-dG or poly-dC single-stranded DNA sequence, or pharmaceutically acceptable salt thereof of any one of embodiments 1-56 and an antigen or a nucleic acid sequence encoding the antigen.
76. The kit of embodiment 75, wherein the antigen is an influenza antigen, or fragment thereof.
77. The kit of embodiment 76, wherein the antigen is an influenza nucleoprotein, or fragment thereof.
78. The kit of embodiment 77, wherein the antigen is a coronavirus antigen, or fragment thereof.

### OTHER EMBODIMENTS

Various modifications and variations of the described compositions, methods, and uses of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

1. A compound comprising a poly-deoxyadenosine (poly-dA) nucleic acid sequence and an albumin-binding domain, or a pharmaceutically acceptable salt thereof.

2. A compound comprising a poly-deoxythymidine (poly-dT) nucleic acid sequence and an albumin binding moiety, or a pharmaceutically acceptable salt thereof.

3. The compound or pharmaceutically acceptable salt thereof of claim 1 or claim 2, wherein the compound or pharmaceutically acceptable salt thereof comprises a poly-dA nucleic acid sequence and a poly-dT nucleic acid sequence.

4. The compound or pharmaceutically acceptable salt thereof of claim 3, wherein the poly-dA nucleic acid sequence and the poly-dT nucleic acid sequence hybridize to form a double-stranded DNA sequence.

5. The compound or pharmaceutically acceptable salt thereof of claim 1 or 2, wherein the poly-dA nucleic acid sequence and/or poly-dT nucleic acid sequence comprise between 30 and 100 nucleotides.

6. The compound or pharmaceutically acceptable salt thereof of claim 5, wherein the poly-dA nucleic acid sequence and/or poly-dT nucleic acid sequence comprise between 50 and 100 nucleotides.

7. The compound or pharmaceutically acceptable salt thereof of claim 5, wherein the poly-dA nucleic acid sequence and/or poly-dT nucleic acid sequence comprise between 30 and 50 nucleotides.

8. The compound or pharmaceutically acceptable salt thereof of claim 5, wherein the poly-dA nucleic acid sequence and/or poly-dT nucleic acid sequence comprise 30, 40, 50, 75, or 100 nucleotides.

9. The compound or pharmaceutically acceptable salt thereof of claim 3, wherein the poly-dA nucleic acid sequence and poly-dT nucleic acid sequence comprise the same number of nucleotides.

10. The compound or pharmaceutically acceptable salt thereof of claim 1 or 2, wherein the poly-dA nucleic acid sequence and/or poly-dT nucleic acid sequence comprises a mixture of dA and dT nucleic acid residues.

11. The compound or pharmaceutically acceptable salt thereof of claim 10, wherein the poly-dA nucleic acid sequence comprises between 100% and 51% dA nucleic acid residues and between 0% and 49% dT nucleic acid residues.

12. The compound or pharmaceutically acceptable salt thereof of claim 10, wherein the poly-dT nucleic acid sequence comprises between 100% and 51% dT nucleic acid residues and between 0% and 49% dA nucleic acid residues.

13. The compound or pharmaceutically acceptable salt thereof of claim 1 or 2, wherein at least one internucleotide group connecting the nucleotides in the poly-dA nucleic acid sequence and poly-dT nucleic acid sequence is a phosphodiester.

14. The compound or pharmaceutically acceptable salt thereof of claim 1 or 2, wherein all internucleotide groups connecting the nucleotides in the poly-dA nucleic acid sequence and poly-dT nucleic acid sequence are phosphorothioate.

15. The compound or pharmaceutically acceptable salt thereof of claim 1 or 2, wherein the albumin-binding domain is bonded to the 5′ end of the poly-dA nucleic acid sequence.

16. The compound or pharmaceutically acceptable salt thereof of claim 1 or 2, wherein the albumin-binding domain is bonded to the 5′ end of the poly-dT nucleic acid sequence.

17. A compound comprising an interferon stimulatory DNA (ISD) sequence and an albumin-binding domain or an immunostimulatory herpes simplex virus (HSV) sequence and an albumin-binding domain, or a pharmaceutically acceptable salt thereof.

18. The compound or pharmaceutically acceptable salt thereof of claim 17, wherein the albumin-binding domain is bonded to the 5′ end of the ISD sequence or the immunostimulatory HSV sequence.

19. The compound or pharmaceutically acceptable salt thereof of claim 1, 2, 17, or 18, wherein the albumin-binding is a lipid.

20. The compound or pharmaceutically acceptable salt thereof of claim 19, wherein the lipid is a diacyl lipid.

21. The compound or pharmaceutically acceptable salt thereof of claim 20, wherein the diacyl lipid comprises acyl chains comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29, or 30 hydrocarbon units.

22. The compound or pharmaceutically acceptable salt thereof of claim 20, wherein the lipid is 1,2-distearyl-sn-glycero-3-phosphoethanolamine (DSPE).

23. The compound or pharmaceutically acceptable salt thereof of claim 20, wherein the poly-dA nucleic acid sequence, the poly-dT nucleic acid sequence, or the ISD sequence is bonded or linked by a linker to the following lipid: 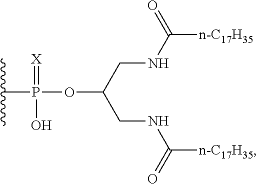
or a salt thereof,
wherein X is O or S.

24. The compound or pharmaceutically acceptable salt thereof of claim 23, wherein the linker is selected from the group consisting of a hydrophilic polymer, a string of hydrophilic amino acids, a polysaccharide, and an oligonucleotide, or a combination thereof.

25. The compound or pharmaceutically acceptable salt thereof of claim 23, wherein the linker comprises “N” polyethylene glycol units, wherein N is between 24-50.

26. The compound or pharmaceutically acceptable salt thereof of claim 25, wherein the linker comprises PEG24-amido-PEG24.

27. A poly-dA and poly-dT double-stranded DNA sequence comprising between 30 and 100 paired nucleotides.

28. The poly-dA and poly-dT double stranded DNA sequence of claim 27, wherein the poly-dA and poly-dT comprise the same number of nucleotides, or a pharmaceutically acceptable salt thereof.

29. A poly-dA or poly-dT single-stranded DNA sequence comprising between 30 and 100 nucleotides, or a pharmaceutically acceptable salt thereof.

30. A compound comprising a poly-deoxyguanosine (poly-dG) nucleic acid sequence nucleic acid sequence and an albumin-binding domain, or a pharmaceutically acceptable salt thereof.

31. A compound comprising a poly-deoxycytosine (poly-dC) nucleic acid sequence and an albumin binding moiety, or a pharmaceutically acceptable salt thereof.

32. The compound or pharmaceutically acceptable salt thereof of claim 30 or claim 31, wherein the compound or pharmaceutically acceptable salt comprises a poly-dG nucleic acid sequence and a poly-dC nucleic acid sequence.

33. The compound or pharmaceutically acceptable salt thereof of claim 32, wherein the poly-dG nucleic acid sequence and the poly-dC nucleic acid sequence hybridize to form a double-stranded DNA sequence.

34. The compound or pharmaceutically acceptable salt thereof of claim 30 or 31, wherein the poly-dG nucleic acid sequence and/or poly-dC nucleic acid sequence comprise between 30 and 100 nucleotides.

35. The compound or pharmaceutically acceptable salt thereof of claim 34, wherein the poly-dG nucleic acid sequence and/or poly-dC nucleic acid sequence comprise between 50 and 100 nucleotides.

36. The compound or pharmaceutically acceptable salt thereof of claim 34, wherein the poly-dG nucleic acid sequence and/or poly-dC nucleic acid sequence comprise between 30 and 50 nucleotides.

37. The compound or pharmaceutically acceptable salt thereof of claim 34, wherein the poly-dG nucleic acid sequence and/or poly-dC nucleic acid sequence comprise 30, 40, 50, 75, or 100 nucleotides.

38. The compound or pharmaceutically acceptable salt thereof of claim 32, wherein the poly-dG nucleic acid sequence and poly-dC nucleic acid sequence comprise the same number of nucleotides.

39. The compound or pharmaceutically acceptable salt thereof of claim 30 or 31, wherein the poly-dG nucleic acid sequence and/or poly-dC nucleic acid sequence comprises a mixture of dG and dC nucleic acid residues.

40. The compound or pharmaceutically acceptable salt thereof of claim 39, wherein the poly-dG nucleic acid sequence comprises between 100% and 51% dG nucleic acid residues and between 0% and 49% dC nucleic acid residues.

41. The compound or pharmaceutically acceptable salt thereof of claim 39, wherein the poly-dC nucleic acid sequence comprises between 100% and 51% dC nucleic acid residues and between 0% and 49% dG nucleic acid residues.

42. The compound or pharmaceutically acceptable salt thereof of claim 30 or 31, wherein at least one internucleotide group connecting the nucleotides in the poly-dG nucleic acid sequence and poly-dC nucleic acid sequence is a phosphodiester.

43. The compound or pharmaceutically acceptable salt thereof of claim 30 or 31, wherein all internucleotide groups connecting the nucleotides in the poly-dG nucleic acid sequence and poly-dC nucleic acid sequence are phosphorothioate.

44. The compound or pharmaceutically acceptable salt thereof of claim 30 or 31, wherein the albumin-binding domain is bonded to the 5′ end of the poly-dG nucleic acid sequence.

45. The compound or pharmaceutically acceptable salt thereof of claim 30 or 31, wherein the albumin-binding domain is bonded to the 5′ end of the poly-dC nucleic acid sequence.

46. The compound or pharmaceutically acceptable salt thereof of claim 30 or 31, wherein the albumin-binding is a lipid.

47. The compound or pharmaceutically acceptable salt thereof of claim 46, wherein the lipid is a diacyl lipid.

48. The compound or pharmaceutically acceptable salt thereof of claim 47, wherein the diacyl lipid comprises acyl chains comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29, or 30 hydrocarbon units.

49. The compound or pharmaceutically acceptable salt thereof of claim 47, wherein the lipid is 1,2-distearoyl-sn-glycero-3-phosphoethanolarnine (DSPE).

50. The compound or pharmaceutically acceptable salt thereof of claim 47, wherein the poly-dG nucleic acid sequence and/or the poly-dC nucleic acid sequence is bonded or linked by a linker to the following lipid: 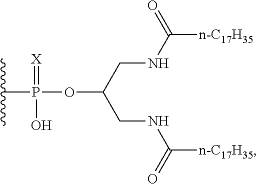
or a salt thereof,
wherein X is O or S.

51. The compound or pharmaceutically acceptable salt thereof of claim 50, wherein the linker is selected from the group consisting of a hydrophilic polymer, a string of hydrophilic amino acids, a polysaccharide, and an oligonucleotide, or a combination thereof.

52. The compound or pharmaceutically acceptable salt thereof of claim 51, wherein the linker comprises “N” polyethylene glycol units, wherein N is between 24-50.

53. The compound or pharmaceutically acceptable salt thereof of claim 52 wherein the linker comprises PEG24-amido-PEG24.

54. A poly-dG and poly-dC double-stranded DNA sequence comprising between 30 and 100 paired nucleotides.

55. The poly-dG and poly-dC double stranded DNA sequence of claim 54, wherein the poly-dG and poly-dC comprise the same number of nucleotides, or a pharmaceutically acceptable salt thereof.

56. A poly-dG or poly-dC single-stranded DNA sequence comprising between 30 and 100 nucleotides, or a pharmaceutically acceptable salt thereof.

57. A method of inducing an immune response against an antigen in a subject, the method comprising administering a compound, poly-dA and poly-dT double-stranded DNA sequence, poly-dA or poly-dT single-stranded DNA sequence, poly-dG and poly-dC double-stranded DNA sequence, poly-dG or poly-dC single-stranded DNA sequence, or pharmaceutically acceptable salt thereof of any one of claims 1, 2, 17, 18, 27-31, and 54-56 and an antigen to the subject.

58. The method of claim 57, further comprising administering an adjuvant to the subject.

59. The method of claim 57, wherein the antigen is an influenza antigen, or fragment thereof.

60. The method of claim 59, wherein the antigen is an Influenza nucleoprotein, or fragment thereof.

61. The method of claim 60, wherein the Influenza nucleoprotein comprises a polypeptide sequence having at least 85% sequence identity to SEQ ID NO: 22.

62. The method of claim 61, wherein the Influenza nucleoprotein comprises a polypeptide sequence having at least 95% sequence identity to SEQ ID NO: 22.

63. The method of claim 62, wherein the Influenza nucleoprotein comprises a polypeptide sequence of SEQ ID NO: 22.

64. The method of claim 57, wherein the antigen is a coronavirus antigen, or fragment thereof.

65. The method claim 64, wherein the antigen is a coronavirus spike protein, or fragment thereof.

66. The method of claim 64, wherein the antigen is a coronavirus nucleocapsid protein, or fragment thereof.

67. The method of claim 57, wherein the compound or pharmaceutically acceptable salt thereof of any one of claims 1-56 is administered subcutaneously.

68. The method of claim 57, the antigen is administered intramuscularly, subcutaneously, intravenously, intraperitoneally, topically, or orally.

69. The method of claim 57, wherein the subject is a mammal.

70. The method of claim 67, wherein the subject is a human.

71. A pharmaceutical composition comprising the compound, poly-dA and poly-dT double-stranded DNA sequence, poly-dA or poly-dT single-stranded DNA sequence, poly-dG and poly-dC double-stranded DNA sequence, poly-dG or poly-dC single-stranded DNA sequence, or pharmaceutically acceptable salt thereof of any one of claims 1, 2, 17, 18, 27-31, and 54-56 and an antigen, or a nucleic acid sequence encoding the antigen, and a pharmaceutically acceptable carrier.

72. The pharmaceutical composition of claim 71, wherein the antigen is an influenza antigen or fragment thereof.

73. The pharmaceutical composition of claim 72, wherein the antigen is an influenza nucleoprotein or fragment thereof.

74. The pharmaceutical composition of claim 71, wherein the antigen is a coronavirus antigen, or fragment thereof.

75. A kit comprising a compound, poly-dA and poly-dT double-stranded DNA sequence, poly-dA or poly-dT single-stranded DNA sequence, poly-dG and poly-dC double-stranded DNA sequence, poly-dG or poly-dC single-stranded DNA sequence, or pharmaceutically acceptable salt thereof of any one of claims 1, 2, 17, 18, 27-31, and 54-56 and an antigen or a nucleic acid sequence encoding the antigen.

76. The kit of claim 75, wherein the antigen is an influenza antigen, or fragment thereof.

77. The kit of claim 76, wherein the antigen is an influenza nucleoprotein, or fragment thereof.

78. The kit of claim 77, wherein the antigen is a coronavirus antigen, or fragment thereof.


SEQUENCE LISTING (37 sequences)

SEQ ID NO: 1 - protein, 4 aa, synthetic construct
GGGA

SEQ ID NO: 2 - protein, 4 aa, synthetic construct
GGGG

SEQ ID NO: 3 - protein, 5 aa, synthetic construct
GGGAG

SEQ ID NO: 4 - protein, 6 aa, synthetic construct
GGGAGG

SEQ ID NO: 5 - protein, 7 aa, synthetic construct
GGGAGGG

SEQ ID NO: 6 - protein, 4 aa, synthetic construct
GGAG

SEQ ID NO: 7 - protein, 4 aa, synthetic construct
GGSG

SEQ ID NO: 8 - protein, 4 aa, synthetic construct
AGGG

SEQ ID NO: 9 - protein, 4 aa, synthetic construct
SGGG

SEQ ID NO: 10 - protein, 6 aa, synthetic construct
GGAGGA

SEQ ID NO: 11 - protein, 6 aa, synthetic construct
GGSGGS

SEQ ID NO: 12 - protein, 9 aa, synthetic construct
GGAGGAGGA

SEQ ID NO: 13 - protein, 9 aa, synthetic construct
GGSGGSGGS

SEQ ID NO: 14 - protein, 12 aa, synthetic construct
GGAGGAGGAGGA

SEQ ID NO: 15 - protein, 12 aa, synthetic construct
GGSGGSGGSGGS

SEQ ID NO: 16 - protein, 8 aa, synthetic construct
GGAGGGAG

SEQ ID NO: 17 - protein, 8 aa, synthetic construct
GGSGGGSG

SEQ ID NO: 18 - protein, 12 aa, synthetic construct
GGAGGGAGGGAG

SEQ ID NO: 19 - protein, 12 aa, synthetic construct
GGSGGGSGGGSG

SEQ ID NO: 20 - protein, 15 aa, synthetic construct
GGGGAGGGGAGGGGA

SEQ ID NO: 21 - protein, 15 aa, synthetic construct
GGGGSGGGGSGGGGS

SEQ ID NO: 22 - protein, 498 aa, Influenza A virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDN

SEQ ID NO: 23 - DNA, 45 bp, synthetic construct
  source 1..45: mol_type = other DNA
tacagatctactagtgatctatgactgatctgtacatgatctaca

SEQ ID NO: 24 - DNA, 45 bp, synthetic construct
  source 1..45: mol_type = other DNA
atgtctagatgatcactagatactgactagacatgtactagatgt

SEQ ID NO: 25 - protein, 8 aa, synthetic construct
VNFNFNGL

SEQ ID NO: 26 - DNA, 24 bp, synthetic construct
  source 1..24: mol_type = other DNA
tcgtcgttttgtcgttttgtcgtt

SEQ ID NO: 27 - protein, 8 aa, synthetic construct
VNFNFNGL

SEQ ID NO: 28 - protein, 17 aa, synthetic construct
ISQAVHAAHAEINEAGR

SEQ ID NO: 29 - protein, 8 aa, synthetic construct
SIINFEKL

SEQ ID NO: 30 - DNA, 50 bp, synthetic construct
  source 1..50: mol_type = other DNA
aaaaaaaaaaaaaaaaaaaaaaaaaaaaaaaaaaaaaaaaaaaaaaaaaa

SEQ ID NO: 31 - DNA, 50 bp, synthetic construct; identical to SEQ ID NO: 30
  source 1..50: mol_type = other DNA
  modified_base 1: mod_base = OTHER; note = Adenosine is modified with an amphiphilic lipid

SEQ ID NO: 32 - DNA, 50 bp, synthetic construct
  source 1..50: mol_type = other DNA
tttttttttttttttttttttttttttttttttttttttttttttttttt

SEQ ID NO: 33 - DNA, 50 bp, synthetic construct; identical to SEQ ID NO: 32
  source 1..50: mol_type = other DNA
  modified_base 1: mod_base = OTHER; note = Thymine is modified with an amphiphilic lipid

SEQ ID NO: 34 - DNA, 60 bp, synthetic construct
  source 1..60: mol_type = other DNA
taagacacgatgcgataaaatctgtttgtaaaatttattaagggtacaaattgccctagc

SEQ ID NO: 35 - DNA, 60 bp, synthetic construct
  source 1..60: mol_type = other DNA
gctagggcaatttgtacccttaataaattttacaaacagattttatcgcatcgtgtctta

SEQ ID NO: 36 - DNA, 50 bp, synthetic construct
  source 1..50: mol_type = other DNA
atatatatatatatatatatatatatatatatatatatatatatatatat

SEQ ID NO: 37 - DNA, 50 bp, synthetic construct; identical to SEQ ID NO: 36
  source 1..50: mol_type = other DNA
  modified_base 1: mod_base = OTHER; note = Adenosine is modified with an amphiphilic lipid